SEQUENCE LISTING (6 sequences)

SEQ ID NO: 1 - protein, 29 aa, Artificial Sequence
  Synthetic
MEASPASGPRHLMDPHIFTSNFNNGIGRH

SEQ ID NO: 2 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 3 - protein, 382 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 4 - protein, 1251 aa, Homo sapiens
MRRGGWRKRAENDGWETWGGYMAAKVQKLEEQFRSDAAMQKDGTSSTIFSGVAIYVNGYTDPSAEELRKLMMLHGGQYHVYYSRSKTTHIIATNLPNAKIKELKGEKVIRPEWIVESIKAGRLLSYIPYQLYTKQSSVQKGLSFNPVCRPEDPLPGPSNIAKQLNNRVNHIVKKIETENEVKVNGMNSWNEEDENNDFSFVDLEQTSPGRKQNGIPHPRGSTAIFNGHTPSSNGALKTQDCLVPMVNSVASRLSPAFSQEEDKAEKSSTDFRDCTLQQLQQSTRNTDALRNPHRTNSFSLSPLHSNTKINGAHHSTVQGPSSTKSTSSVSTFSKAAPSVPSKPSDCNFISNFYSHSRLHHISMWKCELTEFVNTLQRQSNGIFPGREKLKKMKTGRSALVVTDTGDMSVLNSPRHQSCIMHVDMDCFFVSVGIRNRPDLKGKPVAVTSNRGTGRAPLRPGANPQLEWQYYQNKILKGKAADIPDSSLWENPDSAQANGIDSVLSRAEIASCSYEARQLGIKNGMFFGHAKQLCPNLQAVPYDFHAYKEVAQTLYETLASYTHNIEAVSCDEALVDITEILAETKLTPDEFANAVRMEIKDQTKCAASVGIGSNILLARMATRKAKPDGQYHLKPEEVDDFIRGQLVTNLPGVGHSMESKLASLGIKTCGDLQYMTMAKLQKEFGPKTGQMLYRFCRGLDDRPVRTEKERKSVSAEINYGIRFTQPKEAEAFLLSLSEEIQRRLEATGMKGKRLTLKIMVRKPGAPVETAKFGGHGICDNIARTVTLDQATDNAKIIGKAMLNMFHTMKLNISDMRGVGIHVNQLVPTNLNPSTCPSRPSVQSSHFPSGSYSVRDVFQVQKAKKSTEEEHKEVFRAAVDLEISSASRTCTFLPPFPAHLPTSPDTNKAESSGKWNGLHTPVSVQSRLNLSIEVPSPSQLDQSVLEALPPDLREQVEQVCAVQQAESHGDKKKEPVNGCNTGILPQPVGTVLLQIPEPQESNSDAGINLIALPAFSQVDPEVFAALPAELQRELKAAYDQRQRQGENSTHQQSASASVPKNPLLHLKAAVKEKKRNKKKKTIGSPKRIQSPLNNKLLNSPAKTLPGACGSPQKLIDGFLKHEGPPAEKPLEELSASTSGVPGLSSLQSDPAGCVRPPAPNLAGAVEFNDVKTLLREWITTISDPMEEDILQVVKYCTDLIEEKDLEKLDLVIKYMKRLMQQSVESVWNMAFDFILDNVQVVLQQTYGSTLKVT

SEQ ID NO: 5 - DNA, 40 bp, Artificial sequence
  Synthetic
attattattattattattatttcatttatttatttattta

SEQ ID NO: 6 - DNA, 40 bp, Artificial sequence
  Synthetic
attattattattgcaagctgttcagcttgctgaatttatt

(19) United States
(12) Patent Application Publication
(10) Pub. No.: US 2024/0309457 A1
(43) Pub. Date: Sep. 19, 2024

(54) TRACKING APOBEC MUTATIONAL SIGNATURES IN TUMOR CELLS

(71) Applicants: THE BROAD INSTITUTE, INC., Cambridge, MA (US); MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR THE TREATMENT OF CANCER AND ALLIED DISEASES, New York, NY (US); GENOME RESEARCH LIMITED, Cambridge (GB)

(72) Inventors: Mia Petljak, Cambridge, MA (US); Michael R. Stratton, London (GB); John Maciejowski, New York, NY (US)

(73) Assignees: The Broad Institute, Inc., Cambridge, MA (US); Memorial Sloan-Kettering Cancer Center, New York, NY (US); Genome Research Limited, Hinxton (GB); Sloan-Kettering Institute for Cancer Research, New York, NY (US); Memorial Hospital for the Treatment of Cancer and Allied Diseases, New York, NY (US)

(21) Appl. No.: 18/273,715

(22) PCT Filed: Jan. 21, 2022
(86) PCT No.: PCT/US2022/013328

§ 371 Date: Jul. 21, 2023

Related U.S. Application Data
(60) Provisional application No. 63/140,706, filed on Jan. 22, 2021.

(51) Int. Cl. C12Q 1/6886; C07K 16/40; C12N 5/09; C12N 15/113; G01N 33/50

(52) U.S. Cl. CPC C12Q 1/6886; C07K 16/40; C12N 5/0693; C12N 15/1137; G01N 33/5011; C12N 2310/14; C12N 2510/00; C12Q 2600/106; C12Q 2600/156

(57) ABSTRACT

The present disclosure provides methods for treating cancer in a subject (by inhibiting e.g., APOBEC3A, APOBEC3B, or REV1), and methods of diagnosing cancer in a subject. Methods of tracking mutagenesis induced by a gene of interest (e.g., APOBEC3A, APOBEC3B, or REV1) and methods of screening for inhibitors and synthetic lethalities are also described herein. Further provided by the present disclosure are cell lines and antibodies for use in the methods described herein.

### RELATED APPLICATIONS

This application claims priority under 35 U.S.C. § 119(e) to U.S. Provisional Application, U.S. Ser. No. 63/140,706, filed Jan. 22, 2021, which is incorporated herein by reference.

### GOVERNMENT SUPPORT

This invention was made with government support under Grant No. R01ES030993-01A1, R01ES032547-01, R00CA212290, and P30CA008748 awarded by the National Institutes of Health. The government has certain rights in the invention.

### BACKGROUND OF THE INVENTION

Many cancer cells undergo continuous mutation that can help tumors evade therapeutic interventions and significantly impact disease progression. New and improved methods are needed to conclusively identify the various pathways that drive cancer mutagenesis, and to specifically quantify the extent to which different mutagenic pathways are active in tumor cells.

### SUMMARY OF THE INVENTION

Tumor cell mutagenesis presents a complex web of DNA mutations with multiple pathways and targets implicated in mutagenesis. The present disclosure provides a strategy and approach for systematically identifying and quantifying the mutagenic contribution of a given mutagenic pathway (e.g., the APOBEC family of cytidine deaminases). Using this approach, one can define the mutagenic features and trends in a specific tumor cell type or cell line and track the time course of tumor mutagenesis as defined by which specific pathways are active at a given point in time. The observed data can then be cross-referenced with broader trends in clinical tumor progression and used to guide treatment and inform cancer diagnosis and prognosis. As disclosed herein, APOBEC3A, APOBEC3B, and REV1 have been identified as a driver of specific mutagenic signatures in tumor cells.

The increase in whole-genome sequencing throughput over the last decade has enabled systematic investigations into the patterns of somatic mutations in genomes (e.g., genomes of healthy cells and genomes of cancer cells) at high resolution. Such efforts revealed both unclustered and clustered mutations at cytosine bases commonly present at TCN (where N is any base) trinucleotide sequence contexts in human cancers (Nik-Zainal et al. 2012; Roberts et al. 2012). Previously recognized sequence preferences of the APOBEC3 family of cytidine deaminases, which target DNA and RNA of viruses and retroelements at TCN sequence contexts as part of the innate immune defense, led to the proposal that certain observed mutations may arise due to APOBEC3 off-target activity (Nik-Zainal et al. 2012; Roberts et al. 2012). The APOBEC3 family, which evolved to edit viral sequences, thus emerged as a putative double-edge sword lingering in human cells, but might sometimes faultily unleash its mutagenic effects on the human genome.

Subsequent mathematical deconvolution of somatic mutational patterns across thousands of human cancer genomes led to the identification of APOBEC3-associated mutational signatures in more than 75% of cancer types and more than 50% of all cancer genomes analyzed to date, with a particular prominence in many cancers of unknown etiological origins, such as breast cancer, bladder cancer, lung adenocarcinoma, esophageal adenocarcinoma, and other cancers (Alexandrov et al. 2020; Alexandrov et al. 2013). Mutagenesis by APOBEC3 deaminases thus emerged as a putative source of one of the most prominent mutational processes in cancer.

Two mutational signatures, termed ‘SBS2’ and ‘SBS13’ have been proposed to be caused by off-target (e.g., activity not associated with protection from viral infections) APOBEC3 activity (Alexandrov et al. 2020). SBS2 is characterized specifically by C>T base substitutions at TCN trinucleotides, while SBS13 is characterized by C>T, C>G, and C>A mutations at TCN motifs (FIG. 1A). These signatures consist of mostly unclustered mutations dispersed across the genome and diffusely clustered foci comprising usually up to four base substitutions (phenomenon termed ‘omikli’) (Mas-Ponte and Supek 2020). In contrast, the more densely clustered foci of APOBEC3-associated mutations at TCN contexts, termed ‘kataegis’, are often comprised of 5 or more single base substitutions and have been associated with structural rearrangements (Nik-Zainal et al. 2012; Mas-Ponte and Supek 2020). The spatial distribution of such unclustered and clustered APOBEC3-associated mutations is thought to be caused by APOBEC3 attacks on ssDNA substrates of different lengths. The APOBEC3 hypothesis (FIG. 1A) thus proposes that APOBEC3-associated DNA damage arises when one of the five APOBEC3 enzymes with a preference for editing at the TCN motifs, namely APOBEC3A, APOBEC3B, APOBEC3C, APOBEC3D/E, APOBEC3F, or APOBEC3H, deaminate cytosine base at a TCN motif thus converting it into a uracil base (Petljak and Maciejowski 2020). Subsequent processing of the uracil likely determines the resulting type of a cytosine mutation (i.e., C>A, C>T or C>G; FIG. 1A) (Petljak and Maciejowski 2020; Helleday et al. 2014). Replication across the uracil bases was proposed to give rise to C>T mutations and thus possibly SBS2, while excision of uracil by a glycosylase such as UNG or SMUG1 and downstream processing by the base-excision repair (BER) and translesion polymerases may give rise to C>T, C>G, and C>A mutations and thus a combination of SBS2 and SBS13 (Petljak and Maciejowski 2020). One hypothesis pertaining to the generation of kataegis proposes that such clustered mutations may arise following attack of an APOBEC3 enzyme on shorter ssDNA substrate exposed by resection associated with homology-mediated double strand break repair, given the common co-occurrence of such clustered mutations with structural variants in cancer (Taylor et al. 2013). Alternatively, kataegis may be caused by APOBEC3 editing of ssDNA generated by cytosolic nucleases exposed to chromatin during nuclear envelope rupturing events associated with chromosomal instability or telomere crisis (Maciejowski and Hatch 2020; Maciejowski et al. 2020; Ly et al. 2019). Genome-wide unclustered and diffusely clustered mutations are thought to be induced on longer ssDNA substrates that can be generated by replication, replication stalling, or post-replicative DNA mismatch repair (Mas-Ponte and Supek 2020; Morganella et al. 2016; Seplyarskiy et al. 2016; Haradhvala et al. 2016; Green et al. 2016).

The identity of the APOBEC3 deaminase(s) underlying somatic mutations in cancer is a major ongoing question in the field. Several studies revealed that APOBEC3B mRNA expression is upregulated in various cancers compared to the matched healthy tissues, and that it positively correlates with genome-wide APOBEC3 mutational burden (Burns et al. 2013; Roberts et al. 2013; Burns et al. 2013; de Bruin et al. 2014; Middlebrooks et al. 2016). Based on these insights, these studies concluded that APOBEC3B was the major contributor of mutations in breast and other cancers. The extent to which APOBEC3B generates mutations in cancer was put into question upon finding that a germline APOBEC3B deletion polymorphism associates with an elevated risk of breast cancer in East Asian populations and a higher overall burden of APOBEC3-associated mutations in breast cancers (Nik-Zainal et al. 2014; Long et al. 2013; Komatsu et al. 2008). The deletion polymorphism that effectively deletes the APOBEC3B sequence and fuses the APOBEC3A coding sequence to the 3′UTR of APOBEC3B was subsequently found to stabilize APOBEC3A mRNA (Caval et al. 2014). More recent refinements of expression-based analyses suggested that APOBEC3A is the only APOBEC3 whose expression correlates with APOBEC-induced mutational burden thus nominating it as a more prominent mutator relative to APOBEC3B (Cortez et al. 2019). Consistent with the possibility that APOBEC3A may be a more prominent mutator than APOBEC3B, examination of the extended sequence contexts at which mutations at TCN contexts occur in human cancer genomes revealed that pyrimidine(Y)-preceded TCN (YTCN) contexts preferred by APOBEC3A are more commonly mutated than purine(R)-preceded TCN (RTCN) motifs preferred by APOBEC3B in most cancers (Chan et al. 2015). Similarly, a minor portion of mutations at TCN context often present at hairpin loops, that are hotspot sequences preferably attacked by APOBEC3A, but not APOBEC3B in an in vitro biochemical assay (Buisson et al. 2019). Thus, whereas APOBEC3B was initially proposed as a major cause of mutational signatures in cancer, APOBEC3A has recently been highlighted as possibly a more relevant mutator. A role for APOBEC3H in cancer mutagenesis has also been proposed (Starrett et al. 2016). The identity of the APOBEC3 deaminase(s) responsible for clustered mutagenesis, kataegis, also remain cryptic. In support of a potential role for APOBEC3B, endogenous APOBEC3B was recently identified as the source of kataegis in a non-tumorigenic cell model of telomere crisis (Maciejowski et al. 2015; Maciejowski et al. 2020).

Speculations regarding the contributions of individual APOBEC3 enzymes and subsequent DNA repair and replication mechanisms that contribute different clustered and unclustered APOBEC3-associated mutations in cancer are supported by association-based rather than causal links (Petljak et al. 2019; Petljak and Maciejowski 2020; Granadillo Rodriguez et al. 2020; Green and Weitzman 2019). Experimental confirmation that APOBEC3 deaminases are indeed the mutators in cancer and identification of the relevant mutator is critical to pursuing the proposed therapeutic interventions based on modulating activities of the cryptic mutator APOBEC3 deaminase (Olson et al. 2018; Venkatesan et al. 2018; Swanton et al. 2015; Driscoll et al. 2020; Law et al. 2016; Green et al. 2017; Nikkilä et al. 2017; Buisson et al. 2017). Furthermore, the ability to investigate the mechanisms of APOBEC3 misregulation in cancer, which remain entirely unknown and critical to understanding the source of a large proportion of mutations in many cancers of unknown origins, depends on defining the relevant mutator enzymes.

Progress has been hindered by differences between the human and murine APOBEC3 loci and the lack of the genetically amenable human cancer cell models with naturally occurring APOBEC3-associated mutagenesis (Petljak and Maciejowski 2020). APOBEC3-associated mutational signatures were recently found to continue to be generated in many human cell lines from cancer cell lineages exposed to APOBEC3-associated mutagenesis in the past (Petljak et al. 2019). Unlike other mutational signatures that were acquired continuously over time, APOBEC3-associated mutations were generated in episodic bursts, suggesting that the underlying misregulation occurs intermittently rather than continuously in cancer cells. The episodic nature of APOBEC3-associated mutations was subsequently observed in primary human tissues (Lawson et al. 2020; Yoshida et al. 2020). Cell lines with active episodic APOBEC3-associated mutagenesis are thus expected to retain patterns of regulation operative in primary cancers. The individual roles of APOBEC3A and APOBEC3B enzymes in generating both APOBEC3-associated clustered and unclustered mutations are disclosed herein, as well as the roles of base excision repair (BER) components in generating the APOBEC3-associated mutations in human breast and lymphoma cancer cells.

Accordingly, the present disclosure provides methods for treating cancer in a subject, methods of diagnosing cancer in a subject, methods of determining prognosis of cancer in a subject, methods of tracking mutagenesis induced by a gene of interest, and methods of screening for inhibitors and synthetic lethalities. The present disclosure also provides cell lines and antibodies.

In one aspect, the present disclosure provides methods for treating cancer in a subject in need thereof with an agent. The methods may comprise using the agent to inhibit an APOBEC protein in the subject. In some embodiments, the method comprises inhibiting APOBEC3A. In some embodiments, the method comprises inhibiting APOBEC3B. The methods may also comprise using the agent to inhibit REV1 in a subject (for example, as disclosed in Chatterjee et al., Proc. Nat. Acad. Sci. U.S.A. 2020, 117(46), 28918-28921), since REV1 has been shown to play a role in the generation of APOBEC3-induced non-clustered signatures SBS2 and SBS13, as well as clustered kataegis and omikli events in cancer cell genomes, as described herein. The agent used in the methods described herein may be a small molecule, a protein (e.g., an antibody or fragment thereof), a peptide, or a nucleic acid. In some embodiments, the agent is an mRNA, an antisense RNA, an miRNA, an siRNA, an RNA aptamer, a double stranded RNA (dsRNA), a short hairpin RNA (shRNA), or an antisense oligonucleotide (ASO). In certain embodiments, the agent is an siRNA.

In another aspect, the present disclosure provides methods of identifying a subject in need of a treatment for cancer who is likely to respond to an APOBEC3A inhibitor. The methods may comprise (i) taking a biological sample (e.g., tumor biopsy) from the subject; and (ii) determining whether a mutational signature induced by APOBEC3A is present in the sample. The subject is likely to respond to an APOBEC3A inhibitor if a mutation induced by APOBEC3A is present in the sample. In some embodiments, determining whether a mutation induced by APOBEC3A is present in the sample is accomplished by performing whole-genome sequencing. In some embodiments, determining whether the mutational signature is present is accomplished by whole exome sequencing. Determining whether a mutation induced by APOBEC3A is present in the sample may also be accomplished by (i) providing a set of primers comprising a first primer and a second primer to the sample, wherein the first primer binds to a region of the genome upstream of a mutation induced by APOBEC3A and the second primer binds to a region of the genome downstream of the mutation induced by APOBEC3A; (ii) amplifying the region of the genome between the first primer and the second primer; and (iii) sequencing the amplified region of the genome.

In some embodiments, the mutations induced by APOBEC3A are single base substitutions (SBS). Single base substitutions induced by APOBEC3A include, but are not limited to, SBS1, SBS2, SBS5, SBS8_18_36, and SBS13 as defined, for example, in Petjalk, M. et al. Cell 2019, 176(6), 1282-1294 and Alenandrov et al., Nature 2020, 578(7793), 94-101.

In another aspect, the present disclosure provides methods of tracking mutagenesis induced by a gene of interest (e.g., APOBEC3A, APOBEC3B, REV1) in a population of cells over time. Such methods may comprise the following steps:
(i) knocking out the gene of interest in a cell from the population of cells to create a knockout (KO) cell line;
(ii) selecting a first KO clone from the KO cell line;
(iii) selecting a first wild-type (WT) clone from the population of cells;
(iv) propagating the first WT clone and the first KO clone by cell culture a first time into a first WT population of cells and a first KO population of cells;
(v) selecting a second WT clone and a second KO clone from the first WT population of cells and the first KO population of cells;
(vi) propagating the second WT clone and the second KO clone selected in step (v) by cell culture a second time to produce a second WT population of cells and a second KO population of cells;
(vii) sequencing the DNA of the second WT population of cells and the second KO population of cells; and
(viii) comparing the mutations present in the second WT population of cells and the second KO population of cells.

Knocking out a gene of interest may be accomplished by various genetic methods. In some embodiments, knocking out a gene of interest is accomplished by transfecting a cell from a population of cells with a vector encoding a nuclease. In certain embodiments, the nuclease is a CRISPR-associated nuclease (e.g., Cas9). The vector may also encode a guide RNA (gRNA). In some embodiments, the vector encodes a gRNA, wherein the sequence of a portion of the gRNA is complementary to a portion of the gene of interest.

The present disclosure contemplates the use of the methods disclosed herein for any gene of interest. In some embodiments, the gene of interest is an APOBEC deaminase (e.g., APOBEC3A or APOBEC3B). In certain embodiments, the gene of interest is APOBEC3A. In some embodiments, the gene of interest is REV1.

In another aspect, the present disclosure provides cancer cell lines comprising a population of knockout (KO) cells. The cells may comprise an APOBEC protein KO. In some embodiments, the cancer cell line comprises a population of APOBEC3A KO cells. In some embodiments, the cancer cell line comprises a population of APOBEC3B KO cells. In some embodiments, the cancer cell line comprises a population of REV1 KO cells.

The cancer cell lines of the present disclosure comprise various cell types including, but not limited to, bladder cancer cells, cervical cancer cells, lung cancer cells, head and neck cancer cells, breast cancer cells, esophageal cancer cells, lymphoma cells, oral squamous cell carcinoma cells, uterine cancer cells, ovarian adenocarcinoma cells, pancreatic adenocarcinoma cells, stomach adenocarcinoma cells, or biliary adenocarcinoma cells. In some embodiments, the cells are breast cancer cells (e.g., derived from the human breast cancer cell line BT-474 or MDA-MB-453). In some embodiments, the cells are lymphoma cells (e.g., derived from the human B cell lymphoma cancer cell line BC-1 or JSC-1). In some embodiments, the cells are derived from a sample taken from a patient (e.g., a cancer patient's tumor).

The methods described herein may also be used to track mutagenesis induced by a gene of interest over time.

Another aspect of the present disclosure provides isolated monoclonal antibodies generated from APOBEC protein peptide sequences, e.g., the N-terminal amino acids of APOBEC3A, such as the peptide sequence: MEASPASGPRHLMDPHIFTSNFNNGIGRH (SEQ ID NO: 1). In some embodiments, the antibody is a mouse monoclonal antibody. In some embodiments, the antibody is a humanized antibody. In certain embodiments, the antibody is an anti-APOBEC3A/B/G antibody. In certain embodiments, the antibody is an anti-APOBEC3A antibody.

In another aspect, the present disclosure provides methods for screening for inhibitors of an APOBEC protein (e.g., APOBEC3A or APOBEC3B). The methods may comprise (i) propagating a population of cells in the presence and absence of a candidate APOBEC3A inhibitor; and (ii) determining whether the frequency of a mutational signature induced by APOBEC3A is reduced in the presence of the candidate APOBEC3A inhibitor. The mutational signature induced by APOBEC3A may be single base substitutions (SBS). For example, single base substitutions include, but are not limited to, SBS1, SBS2, SBS5, SBS8_18_36, and SBS13.

Another aspect of the present disclosure provides methods of screening for inhibitors of DNA repair protein REV1 (referred to hereinafter as (“REV1”). The methods may comprise (i) propagating a population of cells in the presence and absence of a candidate APOBEC3A inhibitor; and (ii) determining whether the frequency of a mutational signature induced by REV1 is reduced in the presence of the candidate REV1 inhibitor. In some embodiments, the mutations induced by REV1 are single base substitutions (e.g., any of the single base substitutions disclosed herein).

In another aspect, the present disclosure provides methods for screening for a synthetic lethality associated with active APOBEC3A comprising propagating a population of WT cells and a population of ABOBEC3A KO cells in the presence of an agent capable of inhibiting the activity of a gene of interest. A synthetic lethality is identified when the population of WT cells is able to propagate in the presence of the agent, and the population of APOBEC3A KO cells is not able to propagate in the presence of the agent. The agent may be an inhibitor of a gene of interest. In some embodiments, the inhibitor is a small molecule inhibitor. In some embodiments, the inhibitor is an siRNA inhibitor. In certain embodiments, the agent is a Cas9 nuclease associated with a gRNA, wherein the sequence of a portion of the gRNA is complementary to a portion of the gene of interest.

In other aspects, the present disclosure also provides reagents for performing any of the methods described herein. In some embodiments, the reagents for performing any one of the methods disclosed herein are provided as part of a kit. In some embodiments, the kit further comprises instructions for performing one of the methods disclosed herein. Primers and vectors for performing the methods disclosed herein are also provided by the present disclosure.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1F show the use of human cancer cell lines to investigate the origins of APOBEC3-associated mutagenesis. FIG. 1A provides mechanisms underlying generation of APOBEC3-associated SBS2 and SBS13 mutational signatures in cancer. Each signature is displayed according to the proportion (y-axis) of 96-substitution classes denoted on the horizontal axis and defined by the six shaded and labeled SBS types and 16 possible alphabetically ordered trinucleotide sequence contexts at which each mutation type presents. FIG. 1B shows that the prevalence of SBS2 and SBS13 in sequences from 780 COSMIC cancer cell lines (top panel) and 1843 sequences from PCAWG primary human cancers (bottom panel). Each bar represents a percentage of mutations attributed to mutational signatures shaded according to the legend provided in an individual cell line or a primary cancer sample belonging to a cancer type denoted at the top. Percentages of mutations attributed to signatures other than APOBEC3-associated SBS2 and SBS13 were summed and denoted as “other.” BRCA and DLBC datasets are magnified to show individual cell lines, including those chosen for further study. FIG. 1C shows that the investigated cell lines carry signatures of historic APOBEC3-associated exposures. Each panel is displayed according to the counts (y-axis) of genome-wide 96-substitution classes denoted on the horizontal axis and defined by the six SBS types shaded according to the legend provided and 16 possible alphabetically ordered trinucleotide sequence contexts at which each mutation type presents. FIG. 1D provides a schematic of the experimental design used to track mutation acquisition over controlled in vitro timeframes. Each cell line was subjected to CRISPR-Cas9 knockouts of the candidate genes. Single cells were isolated from each targeted population of cells and grown into parent clones. Parent clones with confirmed knockouts of the candidate genes were continuously propagated in culture for 99-143 days. Following this period, individual cells were isolated from each parent population and grown into ‘daughter’ clones that were expanded for DNA isolation. DNA from parent and daughter clones was subjected to whole-genome sequencing and mutations were identified in each clone. Subtraction of mutations identified in parent clones from mutations present in their relevant daughters reveals mutations acquired during the in vitro timeframes spanning the two cloning events. FIG. 1E provides a sample overview. Individual experiments are denoted under “Cancer Cell Lines,” the numbers of days spanning the two subcloning events during which mutational acquisition was tracked are denoted under “Days Propagated,” and the total number of wild type and knockout (KO) parent and daughter clones subject to whole-genome sequencing (WGS) is provided under “WGS.” FIG. 1F provides profiles of mutational signatures extracted de novo from 815,923 SBS identified across mutational catalogues of 4 stock cell lines and 136 parent and daughter clones. Signature display is as per FIG. 1A. SBS (single base substitution), TLS (translesion synthesis), PCAWG (Pan-Cancer Analysis of Whole Genomes) WGS (whole-genome sequencing). Each signature is displayed according to the percentage (y-axis) of genome-wide 96-substitution classes denoted on horizontal axis.

FIGS. 2A-2T show that APOBEC3 deaminases drive acquisition of SBS2 and SBS13 in human cancer cells. FIGS. 2A-2H show immunoblotting with anti-APOBEC3A/B/G and anti-actin antibodies in the indicated cell lines. In FIGS. 2A, 2C, and 2E, triangles represent (from left to right) 40 μg, 20 μg, 10 μg, and 5 μg of extracts prepared from the indicated cell lines. Note that the anti-APOBEC3 antibody can detect both APOBEC3A and APOBEC3B. Multiple exposures are shown to better depict APOBEC3A and APOBEC3B signals. FIGS. 2I-2L show mutation acquisition in MDA-MB-453 (FIG. 2I), BT-474 (FIG. 2J), JSC-1 (FIG. 2K), and BC-1 (FIG. 2L) cell lines wild-type or CRISPR-Cas9 edited knockout clones as annotated. Each panel is displayed according to the counts (y-axis) of genome-wide 48 cytosine base substitution classes denoted on the horizontal axis and defined by the three SBS types shaded according to the legend provided and 16 possible alphabetically ordered trinucleotide sequence contexts at which each mutation type presents. The panels above the arrows reflect the mutational spectra of individual parent clones from the denoted experiments. Numbers by the arrow annotate the number of days during which mutation acquisition was tracked, while individual panels below the arrow each correspond to a mutational catalogue of a daughter clone and thus mutations acquired de novo during the indicated in vitro timeframe. Additional JSC-1 clones are shown in FIG. 9. FIGS. 2M-2T show the annotation of mutational signatures shaded according to the legend provided (FIGS. 2M, 2O, 2Q, and 2S, bars represent the numbers of base substitutions attributed to mutational signatures in annotated clones) and enrichments of the cytosine mutations at APOBEC-3B preferred RTCA/N and APOBEC3A-preferred YTCA/N sequence contexts (FIGS. 2N, 2P, 2R, and 2T, R is a purine base, Y is a pyrimidine base, N is any base, and mutated bases are underlined) across mutational catalogues of parent and daughter clones from denoted cell lines.

FIGS. 3A-3F shows that base-excision repair plays a critical role in generation of APOBEC3 mutations in cancer. FIGS. 3A-3B show immunoblotting with anti-APOBEC3A/B/G, anti-UNG, anti-REV1, and anti-actin antibodies in the indicated cell lines. Note that the anti-APOBEC3A/B/G monoclonal detects long and short APOBEC3A isoforms. Asterisks mark nonspecific signals. FIGS. 3C and 3E show mutation acquisition in MDA-MB-453 (FIG. 3C) and BT-474 (FIG. 3E) cell lines. Data for wild-type and CRISPR-Cas9 edited knockout clones is shown as annotated. Each panel is displayed according to the counts (y-axis) of genome-wide 48 cytosine base substitution classes denoted on horizontal axis and defined by the three SBS types shaded according to the legend provided and 16 possible alphabetically ordered trinucleotide sequence contexts at which each mutation type presents. The panels above the arrows reflect the mutational spectra of individual parent clones from the denoted experiments. The numbers by the arrow annotate the number of days during which mutation acquisition was tracked, while individual panels below the arrow each correspond to a mutational catalogue of a daughter clone and thus mutations acquired de novo during the indicated in vitro timeframe. FIGS. 3D and 3F show the annotation of mutational signatures shaded according to the legend provided (bars represent the numbers of base substitutions attributed to mutational signatures in annotated clones) across mutational catalogues of parent and daughter clones from MDA-MB-453 (FIG. 3D) and BT-474 (FIG. 3E) and cell lines. Bars represent base substitutions attributed to mutational signatures in annotated clones.

FIGS. 4A-4G show that APOBEC3 deaminases drive acquisition of kataegis in human cancer cells. FIG. 4A provides circos plots depicting mutations acquired in vitro in exemplary BC-1 daughter clones indicated on top. Base substitutions are plotted as dots in rainfall plots (log of the inter-mutation distance). Arrows point to examples of kataegis. Central lines indicate rearrangements (translocations, tandem duplications, inversions, and deletions according to the legend provided). FIG. 4B shows the frequencies of in vitro-acquired kataegis foci across individual daughter clones from experiments and from cell lines indicated at the horizontal axis. FIG. 4C shows the frequencies of mutations in individual kataegis foci identified across daughter clones from experiments and from cell lines indicated at the horizontal axis. FIG. 4D provides graphs where each bar represents a total number of genome-wide rearrangements identified in parent and daughter clones from denoted cell lines and experiments indicated on the horizontal axes. FIG. 4E provides rainfall plots of mutations acquired during the periods of defined in vitro growth in a selection of clones. Each dot represents a single base substitution, according to mutation-type (DBS=double-base substitution). The distances between mutations are plotted on the vertical axes on a log scale. The sample-dependent intermutation distance cutoffs for clustered mutations are shown as solid lines, while regional corrections were performed to account for megabase heterogeneity of mutation rates. Mutation density plots are shown above each rainfall plot depicting the normalized mutation densities across the genome that were used for the regional corrections. FIG. 4F shows the distribution of clustered APOBEC-like mutations (gray; cytosine mutations at TCN contexts) and all other mutations (non-APOBEC like; black), acquired de novo in daughter clones from designated cell lines and experiments. The total clustered tumor mutational burden (TMB) defined as mutations per megabase is further subclassified into the TMB of doublet-base substitutions, omikli associated events, and kataegic events, where each solid bar reflects the median mutational burden for a given set of clones. A Mann-Whitney U test was performed for all statistical comparisons. Types of clustered events across each experiment are shown as bar-plots with each shaded band proportionate to the events observed across all clones. FIG. 4G shows mutation spectra of clustered mutations in non-APOBEC-like contexts acquired de novo in designated clones.

FIGS. 5A-5F show the generation of APOBEC3A and APOBEC3B knockout cell line clones. FIG. 5A provides a schematic of the APOBEC3A locus. The position of exon 3, targeting sgRNAs (sgA3A #1 and sgA3A #2) and primers for PCR screening (JM669 and JM670) are indicated. FIG. 5B shows the PCR amplicons generated using primers JM669 and JM670 and genomic DNA templates prepared from the indicated cell lines. FIG. 5C provides plots depicting the percentage of sequenced amplicons generated as in FIG. 5B that contain deletions (black) or insertions (gray) at the indicated positions. FIG. 5D provides a schematic of the APOBEC3B locus. The position of exons 2-4, targeting sgRNAs (sgA3B #1 and sgA3B #2) and primers for PCR screening (JM663 and JM636) are indicated. FIG. 5E shows PCR amplicons generated using primers JM663 and JM636 and genomic DNA templates prepared from the indicated cell lines. FIG. 5F provides plots depicting the percentage of sequenced amplicons generated as in FIG. 5E that contain deletions (black) and inversions (gray).

FIGS. 6A-6F show the generation of REV1 and UNG knockout cell line clones. FIG. 6A provides a schematic of the UNG locus. The position of exon 1, targeting sgRNAs (sgUNG #1 and sgUNG #2), and primers for PCR screening (JM1093 and JM1094) are indicated. FIG. 6B shows the PCR amplicons generated using primers JM1093 and JM1094 and genomic DNA templates prepared from the indicated cell lines. FIG. 6C provides plots depicting the percentage of sequenced amplicons generated as in FIG. 6B that contain deletions (solid line). FIG. 6D provides a schematic of the REV1 locus. The position of exon 4, targeting sgRNAs (sgREV1 #1 and sgREV1 #2), and primers for PCR screening (KC35 and KC36) are indicated. FIG. 6E shows the PCR amplicons generated using primers KC35 and KC36 and genomic DNA templates prepared from the indicated cell lines. FIG. 6F provides plots depicting the percentage of sequenced amplicons generated as in FIG. 6D that contain deletions (solid line).

FIGS. 7A-7B show population doubling and apoptosis in targeted cancer cell lines. FIG. 7A provides graphs showing the proliferation rates (population doubling measures over successive passages) of the indicated cell lines. The mean and s.d. from n=3 independent biological replicates are shown. FIG. 7B provides plots showing percentages of apoptotic, necrotic, or living cells as indicated by propidium iodide and annexin V staining. Bars represent mean and s.d. from at least two independent experiments.

FIGS. 8A-8M show expression and deamination activities of APOBEC3A and APOBEC3B across cell line clones. FIG. 8A shows the peptides used to generate anti-APOBEC3A/B/G and anti-APOBEC3A mouse monoclonal antibodies. FIG. 8B shows immunoblotting with anti-APOBEC3A, anti-APOBEC3A/B/G, and anti-GFP antibodies in extracts prepared from HEK293T cells transfected with the indicated GFP-APOBEC3 constructs. FIG. 8C shows immunoblotting with anti-APOBEC3A, anti-APOBEC3A/B/G, and anti-actin antibodies in the indicated cell lines. FIGS. 8D-8G show normalized APOBEC3 mRNA levels in the indicated cell lines based on qPCR. The mean and s.d. of n=3 independent biological replicates are shown. FIGS. 8H, 8J, and 8L show cytidine deaminase activity in the indicated cell lines measured against a linear probe after RNase treatment (FIG. 8H), hairpin probe after RNase treatment (FIG. 8J), and hairpin probe after vehicle or RNase treatment (FIG. 8L). FIGS. 8I, 8K, and 8M show measurement of the percentage of processed DNA as in FIGS. 8H, 8J, and 8L.

FIG. 9 provides graphs showing that APOBEC3 deaminases drive acquisition of SBS2 and SBS13 in JSC-1 cell line. Additional clones obtained from JSC-1 experiments are presented in FIG. 2S.

FIG. 10 provides a circos plot-based depiction of mutations identified in individual cell line clones. Circos plots depict mutations acquired in vitro in clones indicated on top. Base substitutions are plotted as dots in rainfall plots (log of inter-mutation distance). Arrows point to examples of kataegis. Central lines indicate rearrangements (translocations, tandem duplications, inversions, and deletions according to the legends provided for each plot).

FIGS. 11A-11E show mutations in individual parent and daughter clones. The distribution and variant allele fractions of mutations identified across all clones are shown. In FIGS. 11A-11D, the left vertical axis depicts the percentage of SBS mutations from BT-474 (FIG. 11A), MDA-MB-453 (FIG. 11B), BC-1 (FIG. 11C) and JSC-1 (FIG. 11D) cell line clones indicated on the right vertical axis that had been identified across the related clones indicated on the horizontal axis upon genotyping of individual mutations. FIG. 11E provides graphs showing the distributions of variant alleles fractions (VAFs) of mutations identified in individual parent and daughter clones from the experiments indicated on top. VAF peaks can sometimes deviate from 50%, as expected for clonal heterozygous somatic mutations in a diploid genome, because cancer cell lines are often polyploid and heterozygous copy number changes across the genome can further modulate the VAF distribution. Bimodal distributions and sub-clonal peaks in wild type clones from the BC-1 cell line likely arose due to sub-clonal evolution of the relevant clones.

FIG. 12 shows analysis of chromosome rearrangements across cell line clones. Plots are provided showing numbers of rearrangement types detected genome-wide in the indicated cell line clones, shaded according to the legend provided.

### DEFINITIONS

Unless defined otherwise, all technical and scientific terms used herein have the meaning commonly understood by a person skilled in the art to which this invention belongs. As used herein, the following terms have the meanings ascribed to them unless specified otherwise.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “administer,” “administering,” or “administration” refers to implanting, absorbing, ingesting, injecting, inhaling, or otherwise introducing an agent or inhibitor as described herein, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

As used herein the term “inhibit” or “inhibition” in the context of enzymes, for example, in the context of APOBEC3A, APOBEC3B, or REV1, refers to a reduction in the activity of the enzyme. In some embodiments, the term refers to a reduction of the level of enzyme activity, e.g., APOBEC3A, APOBEC3B, or REV1 activity, to a level that is statistically significantly lower than an initial level, which may, for example, be a baseline level of enzyme activity. In some embodiments, the term refers to a reduction of the level of enzyme activity, e.g., APOBEC3A, APOBEC3B, or REV1 activity, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of enzyme activity.

The term “sample” or “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. A sample may be taken from a subject, e.g., for diagnostic purposes.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva). In some embodiments, the cancer is bladder cancer, cervical cancer, lung cancer, head and neck cancer, breast cancer, esophageal cancer, lymphoma, oral squamous cell carcinoma, uterine cancer, ovarian adenocarcinoma, pancreatic adenocarcinoma, stomach adenocarcinoma, or biliary adenocarcinoma. In certain embodiments, the cancer is lung cancer. In certain embodiments, lung cancer is lung adenocarcinoma or squamous cell carcinoma. In certain embodiments, the cancer is breast cancer. In certain embodiments, the cancer is B cell lymphoma.

The term “gene” or “gene of interest” refers to a nucleic acid fragment that expresses a protein, including regulatory sequences preceding (5′ non-coding sequences) and following (3′ non-coding sequences) the coding sequence. “Native gene” refers to a gene as found in nature with its own regulatory sequences. “Chimeric gene” or “chimeric construct” refers to any gene or a construct, not a native gene, comprising regulatory and coding sequences that are not found together in nature. Accordingly, a chimeric gene or chimeric construct may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but which is introduced into the host organism by gene transfer. Foreign genes can comprise native genes inserted into a non-native organism, or chimeric genes. A “transgene” is a gene that has been introduced into the genome by a transformation procedure.

An “antibody” refers to a glycoprotein belonging to the immunoglobulin superfamily. The terms antibody and immunoglobulin are used interchangeably. With some exceptions, mammalian antibodies are typically made of basic structural units each with two large heavy chains and two small light chain. There are several different types of antibody heavy chains, and several different kinds of antibodies, which are grouped together into different isotypes based on which heavy chain they possess. Five different antibody isotypes are known in mammals (IgG, IgA, IgE, IgD, and IgM), which perform different roles and help direct the appropriate immune response for each different type of foreign object they encounter. The term “antibody” as used herein also encompasses antibody fragments and nanobodies, as well as variants of antibodies and variants of antibody fragments and nanobodies.

“Small molecules” include molecules, whether naturally occurring or artificially created (e.g., via chemical synthesis) that have a relatively low molecular weight. Typically, a small molecule is an organic compound (e.g., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

Cytidine deaminases are enzymes involved in pyrimidine salvaging. Cytidine deaminases catalyze the irreversible hydrolytic deamination of cytidine and deoxycytidine to uridine and deoxyuridine, respectively. The majority of cytidine deaminases act on RNA, while a few act on DNA (e.g., single stranded DNA). APOBEC proteins are a family of cytidine deaminases. APOBEC proteins include, but are not limited to, APOBEC1, APOEC2, APOEC3A, APOBEC3B, APOBEC3C, APOBEC3D, APOBEC3F, APOBEC3G, APOBEC3H, APOBEC4, and activation-induced deaminase (AID).

The human protein APOBEC3A consists of the amino acid sequence:

(SEQ ID NO: 2)
MEASPASGPRHLMDPHIFTSNFNNGIGRHKTYLCYEVERLDNGTSVKMD
QHRGFLHNQAKNLLCGFYGRHAELRFLDLVPSLQLDPAQIYRVTWFISW
SPCFSWGCAGEVRAFLQENTHVRLRIFAARIYDYDPLYKEALQMLRDAG
AQVSIMTYDEFKHCWDTFVDHQGCPFQPWDGLDEHSQALSGRLRAILQN
QGN.

The human protein APOBEC3B consists of the amino acid sequence:

(SEQ ID NO: 3)
MNPQIRNPMERMYRDTFYDNFENEPILYGRSYTWLCYEVKIKRGRSNLL
WDTGVFRGQVYFKPQYHAEMCFLSWFCGNQLPAYKCFQITWFVSWTPCP
DCVAKLAEFLSEHPNVTLTISAARLYYYWERDYRRALCRLSQAGARVTI
MDYEEFAYCWENFVYNEGQQFMPWYKFDENYAFLHRTLKEILRYLMDPD
TFTFNFNNDPLVLRRRQTYLCYEVERLDNGTWVLMDQHMGFLCNEAKNL
LCGFYGRHAELRFLDLVPSLQLDPAQIYRVTWFISWSPCFSWGCAGEVR
AFLQENTHVRLRIFAARIYDYDPLYKEALQMLRDAGAQVSIMTYDEFEY
CWDTFVYRQGCPFQPWDGLEEHSQALSGRLRAILQNQGN.

The human protein REV1 is a DNA repair protein. REV1 is a Y family DNA polymerase and is sometimes referred to as a deoxycytidyl transferase because it inserts deoxycytidine across from lesions. REV1 uses an arginine as a template, which complements well with cytidine, and thus always adds a cytidine, no matter the nucleotide present at the abasic site. REV1 is thought to play a role in recruiting other TLS proteins. As described herein, REV1 has also been shown to play a role in the in the generation of APOBEC3-induced non-clustered signatures SBS2 and SBS13, as well as clustered kataegis and omikli events in cancer cell genomes.

Human REV1 consists of the amino acid sequence:

(SEQ ID NO: 4)
MRRGGWRKRAENDGWETWGGYMAAKVQKLEEQFRSDAAMQKDGTSSTIF
SGVAIYVNGYTDPSAEELRKLMMLHGGQYHVYYSRSKTTHIIATNLPNA
KIKELKGEKVIRPEWIVESIKAGRLLSYIPYQLYTKQSSVQKGLSFNPV
CRPEDPLPGPSNIAKQLNNRVNHIVKKIETENEVKVNGMNSWNEEDENN
DFSFVDLEQTSPGRKONGIPHPRGSTAIFNGHTPSSNGALKTQDCLVPM
VNSVASRLSPAFSQEEDKAEKSSTDFRDCTLQQLQQSTRNTDALRNPHR
TNSFSLSPLHSNTKINGAHHSTVQGPSSTKSTSSVSTFSKAAPSVPSKP
SDCNFISNFYSHSRLHHISMWKCELTEFVNTLQRQSNGIFPGREKLKKM
KTGRSALVVTDTGDMSVLNSPRHQSCIMHVDMDCFFVSVGIRNRPDLKG
KPVAVTSNRGTGRAPLRPGANPQLEWQYYQNKILKGKAADIPDSSLWEN
PDSAQANGIDSVLSRAEIASCSYEARQLGIKNGMFFGHAKQLCPNLQAV
PYDFHAYKEVAQTLYETLASYTHNIEAVSCDEALVDITEILAETKLTPD
EFANAVRMEIKDQTKCAASVGIGSNILLARMATRKAKPDGQYHLKPEEV
DDFIRGQLVTNLPGVGHSMESKLASLGIKTCGDLQYMTMAKLQKEFGPK
TGQMLYRFCRGLDDRPVRTEKERKSVSAEINYGIRFTQPKEAEAFLLSL
SEEIQRRLEATGMKGKRLTLKIMVRKPGAPVETAKFGGHGICDNIARTV
TLDQATDNAKIIGKAMLNMFHTMKLNISDMRGVGIHVNQLVPTNLNPST
CPSRPSVQSSHFPSGSYSVRDVFQVQKAKKSTEEEHKEVFRAAVDLEIS
SASRTCTFLPPFPAHLPTSPDTNKAESSGKWNGLHTPVSVQSRLNLSIE
VPSPSQLDQSVLEALPPDLREQVEQVCAVQQAESHGDKKKEPVNGCNTG
ILPQPVGTVLLQIPEPQESNSDAGINLIALPAFSQVDPEVFAALPAELQ
RELKAAYDQRQRQGENSTHQQSASASVPKNPLLHLKAAVKEKKRNKKKK
TIGSPKRIQSPLNNKLLNSPAKTLPGACGSPQKLIDGFLKHEGPPAEKP
LEELSASTSGVPGLSSLQSDPAGCVRPPAPNLAGAVEFNDVKTLLREWI
TTISDPMEEDILQVVKYCTDLIEEKDLEKLDLVIKYMKRLMQQSVESVW
NMAFDFILDNVQVVLQQTYGSTLKVT.

### DETAILED DESCRIPTION OF CERTAIN EMBODIMENTS

The APOBEC3 family of cytidine deaminases has emerged as a major putative source of somatic mutations in cancer. However, a lack of appropriate experimental models has hindered establishment of causal links between the activities of individual APOBEC3 enzymes and mutations in cancer cells, leaving the major mutator debatable and the mechanisms underlying different APOBEC3-attributed mutational signatures unknown. To test the long-postulated hypothesis pertaining to APOBEC3 mutagenesis in cancer, candidate APOBEC3 genes were deleted from cancer cell lines that naturally generate APOBEC3-associated mutations in episodic bursts. Deletion of the APOBEC3A paralog severely diminished the acquisition of mutations of speculative APOBEC3 origins in two breast cancer and two lymphoma cell lines, while increased APOBEC3 mutational burdens were observed in APOBEC3B knockout cell lines. APOBEC3A deletion also diminished the appearance of clusters of APOBEC3-associated mutation types, termed kataegis and omikli, which are frequently found in cancer genomes. The uracil glycosylase UNG and the translesion polymerase Rev1 were also found to play critical roles in the generation of mutations induced by APOBEC3A. These data represent the first experimental confirmation that APOBEC3 deaminases generate prevalent clustered and non-clustered mutational signatures in human cancer cells, identify the APOBEC3A and APOBEC3B paralogs as drivers and potential modulators of the episodic mutational bursts, and dissect the roles of the relevant enzymes in generating the associated mutations in breast cancer and B cell lymphoma cell lines. Accordingly, the present disclosure provides methods for treating cancer in a subject, methods of diagnosing cancer in a subject, methods of tracking mutagenesis induced by a gene of interest, and methods of screening for inhibitors and synthetic lethalities. The present disclosure also provides cell lines and antibodies. Finally, the present disclosure additionally provides reagents, kits, primers, and vectors for performing the methods disclosed herein.

### Methods for Treating Cancer

One aspect of the present disclosure provides methods for treating cancer in a subject in need thereof with an agent. The methods may comprise using the agent to inhibit an APOBEC protein in the subject. APOBEC proteins are a family of cytidine deaminases. APOBEC proteins include, but are not limited to, APOBEC1, APOEC2, APOEC3A, APOBEC3B, APOBEC3C, APOBEC3D, APOBEC3F, APOBEC3G, APOBEC3H, APOBEC4, and activation-induced deaminase (AID). In some embodiments, the methods comprise inhibiting APOBEC3A in a subject in need thereof. In some embodiments, the methods comprise inhibiting APOBEC3B in a subject in need thereof. In some embodiments, the methods comprise inhibiting AID in a subject in need thereof. The methods may also comprise using the agent to inhibit the translesion polymerase REV1 in a subject. REV1 is a DNA repair protein in humans. In some embodiments, the methods comprise inhibiting UNG. In certain embodiments, multiple proteins may be inhibited simultaneously, or sequentially (e.g., one or more of APOBEC3A, APOBEC3B, another APOBEC protein, REV1, and UNG are inhibited simultaneously, or sequentially).

The agent used in the methods for treating cancer described herein may be a small molecule. For example, APOBEC inhibitors are described in Olson et al., Cell Chemical Biology 2018, 25(1), 36-49 and Kvach et al., Biochemistry 2019, 58, 391-400. Small molecule inhibitors of APOBEC3A include, for example, those described in King, J. J. et al. ACS Pharmacol. Transl. Sci. 2021, 4(4), 1390-1407, including small molecules with the following structures, and derivatives thereof:

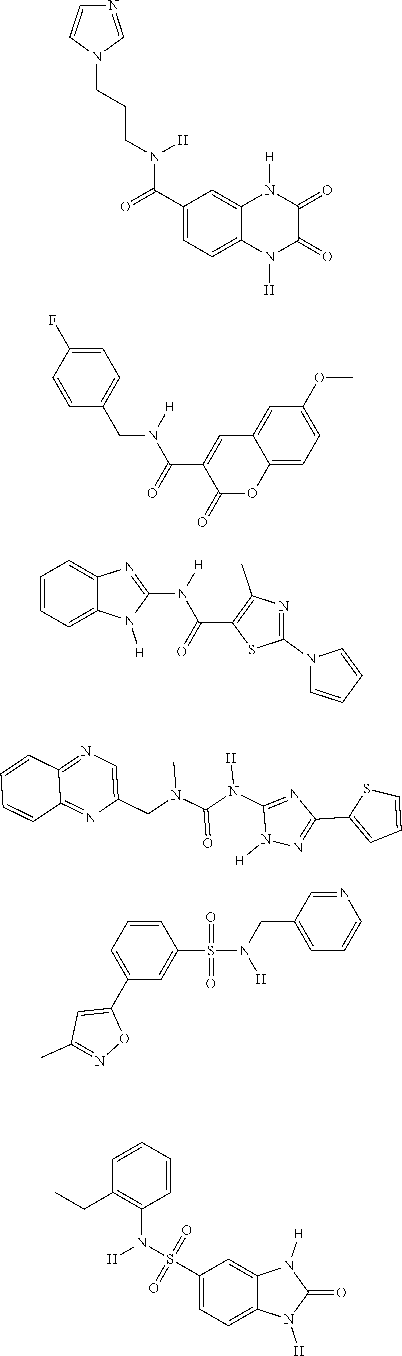

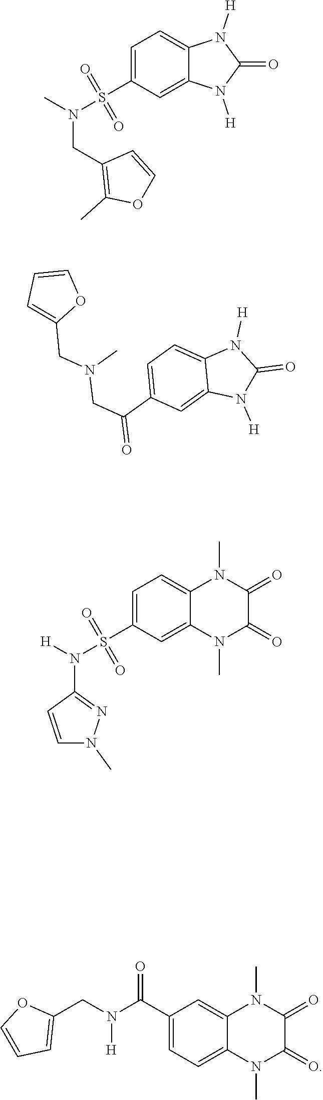 [SMILES: Cc1occc1CN(C)S(=O)(=O)c1ccc2[nH]c(=O)[nH]c2c1 ; CN(CC(=O)c1ccc2[nH]c(=O)[nH]c2c1)Cc1ccco1 ; Cn1ccc(NS(=O)(=O)c2ccc3c(c2)n(C)c(=O)c(=O)n3C)n1 ; Cn1c(=O)c(=O)n(C)c2cc(C(=O)NCc3ccco3)ccc21]

The agent used the methods for treating cancer disclosed herein may also be a protein (including an antibody as described herein). In some embodiments, the inhibitor is an anti-APOBEC3A antibody. In some embodiments, the inhibitor is an anti-APOBEC3B3 antibody. In some embodiments, the antibody is an anti-REV1 antibody.

The agent used in the methods of treating cancer disclosed herein may also be a nucleic acid. In some embodiments, the agent is an mRNA, an antisense RNA, an miRNA, an siRNA, an RNA aptamer, a double stranded RNA (dsRNA), a short hairpin RNA (shRNA), or an antisense oligonucleotide (ASO). In some embodiments, the agent is an siRNA. siRNAs are small inhibitory RNA duplexes that induce the RNA interference (RNAi) pathway, where the siRNA interferes with the expression of specific genes with a complementary nucleotide sequence. siRNA molecules can vary in length (e.g., between 18-30 or 20-25 base pairs) and contain varying degrees of complementarity to their target mRNA in the antisense strand. Some siRNA have unpaired overhanging bases on the 5′ or 3′ end of the sense strand and/or the antisense strand. The term siRNA includes duplexes of two separate strands, as well as single strands that can form hairpin structures comprising a duplex region. For example, an siRNA directed to knocking out APOBEC3A could be used in the treatment of cancer. In some embodiments, the siRNA is directed to knocking out APOBEC3B3. In some embodiments, the siRNA is directed to knocking out REV1. Suitable siRNAs for use in the methods described here include, for example, those disclosed in Cortez, L. M. et al. PLOS Genetics 2019, 15(12):e1008545.

Treatment of any cancer disclosed herein is contemplated by the methods of the present application. In particular, treatment of bladder cancer, cervical cancer, lung cancer, head and neck cancer, breast cancer, esophageal cancer, lymphoma, oral squamous cell carcinoma, uterine cancer, ovarian adenocarcinoma, pancreatic adenocarcinoma, stomach adenocarcinoma, or biliary adenocarcinoma is contemplated by the present disclosure.

Treatment of a subject with any cancer using the methods disclosed herein is contemplated by this disclosure. In some embodiments, subjects with a tumor with one or more mutational signatures associated with an APOBEC protein are treated using the methods disclosed herein. In some embodiments, the tumor in the subject being treated has one or more mutational signatures associated with APOBEC3A. In some embodiments, the one or more mutational signatures are associated with APOBEC3B. In certain embodiments, the one or more mutational signatures are associated with REV1. In certain embodiments, the one or more mutational signatures are associated with UNG.

In another aspect of the present disclosure, methods for treating cancer comprising enhancing the activity of an APOBEC protein are provided. In some embodiments, the activity of APOBEC3B is induced.

Methods for Diagnosing Cancer and/or Identifying a Subject in Need of Cancer Treatment

In another aspect, the present disclosure provides methods of identifying a subject in need of treatment for cancer. The present disclosure contemplates identifying subjects who are likely to respond to or benefit from being treated with an APOBEC3A inhibitor. The methods disclosed herein may comprise (i) taking a sample from the subject (e.g., a tissue biopsy from a tumor); and (ii) determining whether a mutational signature induced by APOBEC3A is present in the sample. The subject is likely to respond to or benefit from treatment with an APOBEC3A inhibitor if a mutational signature induced by APOBEC3A is present in the sample.

Determining whether a mutational signature induced by APOBEC3A is present in the sample may be accomplished through various methods known in the art. For example, determining whether such a mutational signature is present may be determined through any type of sequencing method known in the art, such as whole-genome sequencing. In some embodiments, determining whether the mutational signature is present is accomplished by whole exome sequencing. Determining whether a mutation induced by APOBEC3A is present in the sample may also be accomplished by targeted-gene sequencing (e.g., through a method such as (i) providing a set of primers comprising a first primer and a second primer to the sample, wherein the first primer binds to a region of the genome upstream of a mutational signature induced by APOBEC3A and the second primer binds to a region of the genome downstream of the mutational signature induced by APOBEC3A; (ii) amplifying the region of the genome between the first primer and the second primer; and (iii) sequencing the amplified region of the genome).

Various mutational signatures may be induced by APOBEC3A. For example, a mutational signature induced by APOBEC3A may be a single base substitution (SBS). An SBS is a genetic mutation in which a single nucleotide base is changed from a DNA or RNA sequence in an organism's genome. Single base substitutions induced by APOBEC proteins, such as APOBEC3A, include, but are not limited to, SBS1, SBS2, SBS5, SBS8_18_36, and SBS13. These mutational signatures are known in the art, for example, in Petljak, M. et al. Cell 2019, 176, 1282-1294.

### Methods for Tracking Mutagenesis

Another aspect of the present disclosure provides methods of tracking mutagenesis induced by a gene of interest (e.g., APOBEC3A cytidine deaminase) in a population of cells over time. Such methods may comprise the following steps:
(i) knocking out the gene of interest in a cell from the population of cells to create a knockout (KO) cell line;
(ii) selecting a first KO clone from the KO cell line;
(iii) selecting a first wild-type (WT) clone from the population of cells;
(iv) propagating the first WT clone and the first KO clone by cell culture a first time into a first WT population of cells and a first KO population of cells;
(v) selecting a second WT clone and a second KO clone from the first WT population of cells and the first KO population of cells;
(vi) propagating the second WT clone and the second KO clone selected in step (v) by cell culture a second time to produce a second WT population of cells and a second KO population of cells;
(vii) sequencing the DNA of the second WT population of cells and the second KO population of cells; and
(viii) comparing the mutations present in the second WT population of cells and the second KO population of cells.

Knocking out a gene of interest may be accomplished by any genetic method known in the art. For example, knocking out a gene of interest can be accomplished by transfecting a cell from a population of cells with a vector encoding a nuclease, such as a CRISPR-associated nuclease (e.g., Cas9 nuclease). The vector may also encode a guide RNA (gRNA), where the sequence of a portion of the gRNA is complementary to a portion of the gene of interest (e.g., the sequence has sufficient complementarity to be able to hybridize with the gene of interest, forming a stable duplex). Cells may also be treated with a nuclease and a gRNA directly. Such a treatment may also include a transfection reagent, or a fusion to the nuclease, to help the nuclease and gRNA enter the cell to edit the genome.

In the methods disclosed herein, the first and second propagating steps may each be performed for a variable number of days. For example, the first propagating step may be performed for at least 10 days. The first propagating step may also be performed for at least 15, at least 20, at least 25, at least 30, at least 35, at least 40, at least 45, or at least 50 days. The first propagating step may also be performed for up to 100, up to 150, up to 200, up to 250, or up to 300 days or more. In certain embodiments, the first propagating step is performed for anywhere between 50 and 150 days.

The sequencing step may be performed using various methods known in the art. For example, in some embodiments, the sequencing step comprises whole genome sequencing. In some embodiments, the sequencing step comprises whole exome sequencing. In certain embodiments, the sequencing step comprises targeted gene sequencing.

The present disclosure contemplates the use of the methods disclosed herein for tracking mutagenesis induced by any gene of interest. For example, in some embodiments, the gene of interest is an APOBEC deaminase (e.g., APOBEC3A, APOBEC3B, or any APOBEC deaminase disclosed herein). In certain embodiments, the gene of interest is REV1. In certain embodiments, the gene of interest is UNG.

The present disclosure also provides for distinguishing which APOBEC3A-associated mutational signatures (e.g., SBS's) are diagnostic for APOBEC3A activity, and which mutational signatures are not associated with APOBEC3A activity. Mutational signatures that are not associated with APOBEC3A activity can be used as standards to cross-compare or normalize SBS activity between different cell samples.

### Cell Lines

Another aspect of the present disclosure provides cancer cell lines comprising a population of knockout (KO) cells. The cells may comprise a knockout of an APOBEC protein. The cancer cell lines contemplated by the present disclosure include populations of cells comprising an APOBEC3A KO. In some embodiments, the cancer cell line comprises a population of APOBEC3B KO cells.

The cancer cell lines of the present disclosure comprise various cell types including, but not limited to, bladder cancer cells, cervical cancer cells, lung cancer cells, head and neck cancer cells, breast cancer cells, esophageal cancer cells, lymphoma cells, oral squamous cell carcinoma cells, uterine cancer cells, ovarian adenocarcinoma cells, pancreatic adenocarcinoma cells, stomach adenocarcinoma cells, or biliary adenocarcinoma cells. In some embodiments, the cells are breast cancer cells (e.g., derived from the human breast cancer cell line BT-474 or MDA-MB-453). In some embodiments, the cells are lymphoma cells (e.g., derived from the human B cell lymphoma cancer cell line BC-1 or JSC-1). In some embodiments, the cells are derived from a sample taken from a subject (e.g., a tumor sample).

### Monoclonal Antibodies

Another aspect of the present disclosure provides isolated monoclonal antibodies generated from APOBEC peptides, e.g., the N-terminal amino acids of APOBEC3A, such as the peptide sequence: MEASPASGPRHLMDPHIFTSNFNNGIGRH (SEQ ID NO: 1). The antibodies contemplated by the present disclosure may include any of the several different types of antibody heavy chains, and the several different kinds of antibodies, which are grouped together into different isotypes as disclosed herein. The antibodies disclosed herein may include, for example, any of the five mammalian antibody isotypes (IgG, IgA, IgE, IgD, and IgM).

The present disclosure provides isolated monoclonal antibodies generated from the peptide: MEASPASGPRHLMDPHIFTSNFNNGIGRH (SEQ ID NO: 1). In some embodiments, the monoclonal antibody is a mouse antibody. In some embodiments, the antibody is a human antibody. In some embodiments, the monoclonal antibody is a human antibody. In certain embodiments, the monoclonal antibody is an anti-APOBEC3A/B/G antibody. In certain embodiments, the monoclonal antibody is an anti-APOBEC3A antibody.

### Methods of Screening for Inhibitors

In another aspect, the present disclosure provides methods for screening for inhibitors. For example, methods of screening for inhibitors of an APOBEC protein (e.g., APOBEC3A or APOBEC3B) are provided. Such methods may comprise (i) propagating a population of cells in the presence and absence of a candidate APOBEC3A inhibitor; and (ii) determining whether the frequency of a mutational signature induced by APOBEC3A is reduced in the presence of the candidate APOBEC3A inhibitor. In another example, methods of screening for inhibitors of REV1 are provided. Such methods may comprise (i) propagating a population of cells in the presence and absence of a candidate REV1 inhibitor; and (ii) determining whether the frequency of a mutational signature induced by REV1 is reduced in the presence of the candidate REV1 inhibitor. Inhibitors of other APOBEC family proteins (e.g., APOBEC3B), or inhibitors of UNG, could also be screened for using similar methods.

The mutational signature induced by APOBEC3A may comprise single base substitutions (SBS). For example, single base substitutions include, but are not limited to, SBS1, SBS2, SBS5, SBS8_18_36, and SBS13, as well as any SBS disclosed herein.

### Methods of Screening for Synthetic Lethalities

In another aspect, the present disclosure provides methods for screening for a synthetic lethality associated with active APOBEC3A. A synthetic lethality arises when a combination of deficiencies (e.g., through genetic knockout, or through enzyme inhibition) of at least two genes leads to the death of a cell, while a deficiency of only one of the genes does not result in cell death. Such methods may comprise propagating a population of WT cells and a population of ABOBEC3A KO cells in the presence of an agent capable of inhibiting the activity of a gene of interest. A synthetic lethality is identified when the population of WT cells is able to propagate in the presence of the agent and the population of APOBEC3A KO cells is not able to propagate in the presence of the agent. The agent may be an inhibitor of a gene of interest. In some embodiments, the inhibitor is a small molecule inhibitor, as described herein. In some embodiments, the inhibitor is an siRNA inhibitor, as described herein. In certain embodiments, the agent is a Cas9 nuclease associated with a gRNA, wherein the sequence of a portion of the gRNA is complementary to a portion of the gene of interest, as described herein. The present disclosure also provides similar methods for screening for a synthetic lethality associated with active APOBEC3B, REV1, or UNG.

### Kits, Vectors, Primers, Reagents, Antibodies, and Cell Lines

In other aspects, the present disclosure also provides reagents for performing any of the methods described herein. In some embodiments, the reagents for performing any one of the methods disclosed herein are provided as part of a kit. In some embodiments, the kit further comprises instructions for performing one of the methods disclosed herein. Primers and vectors for performing the methods disclosed herein are also provided by the present disclosure. Additional cell lines and antibodies used in the methods described herein are also provided by this disclosure.

### EXAMPLES

### Example 1: Human Cancer Cell Lines with Active Mutagenesis—Models of APOBEC3 Mutagenesis in Cancer

Mutational signatures identified in a DNA sequence reflect traces of historic mutational processes. It was recently found that cell lines with evidential historic exposure to APOBEC3-associated mutagenesis often continue to generate the unclustered and clustered kataegis mutations associated with APOBEC3 deaminases in episodic bursts over time (Petljak et al. Cell 2019, 176(6), 1282-1294).

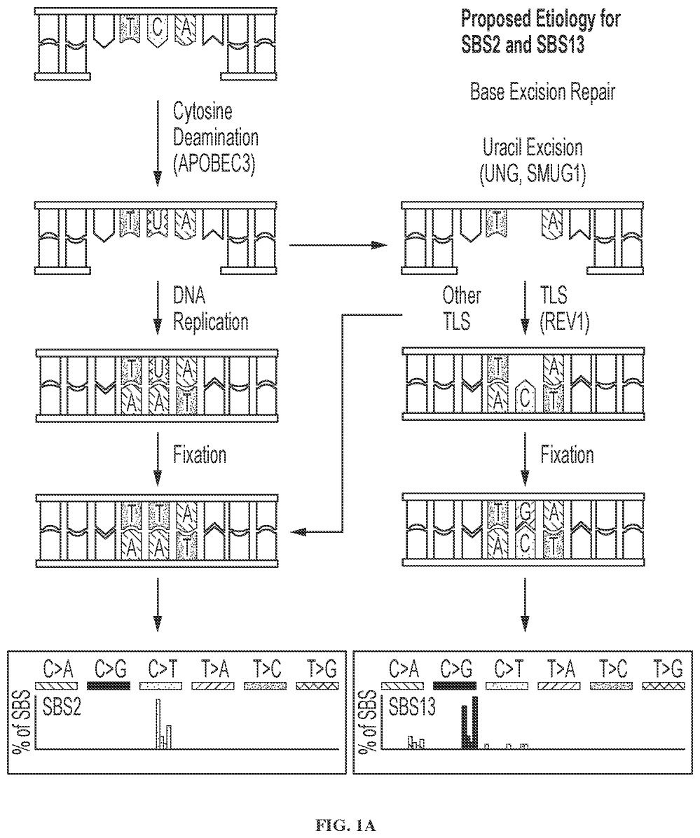

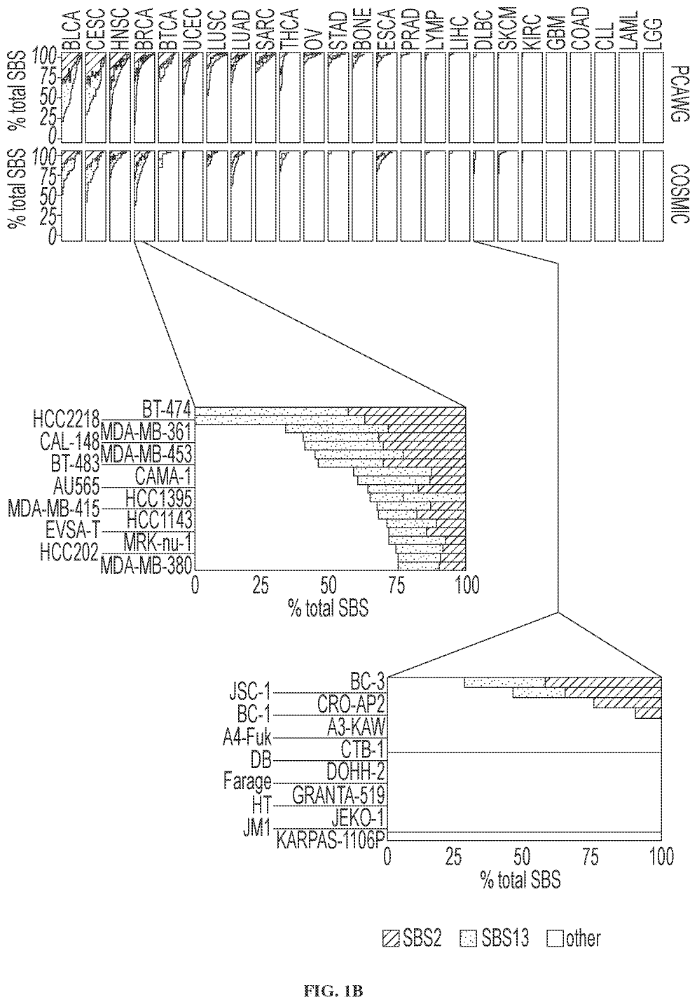

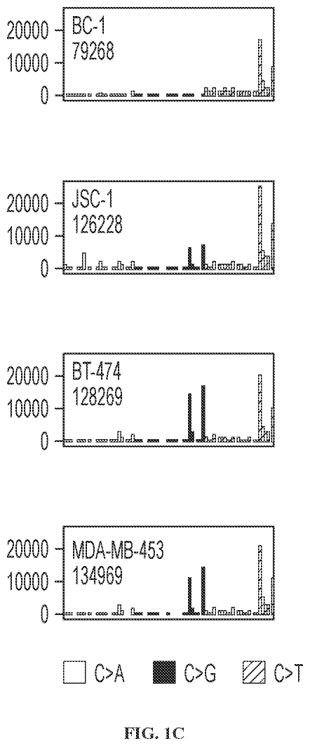

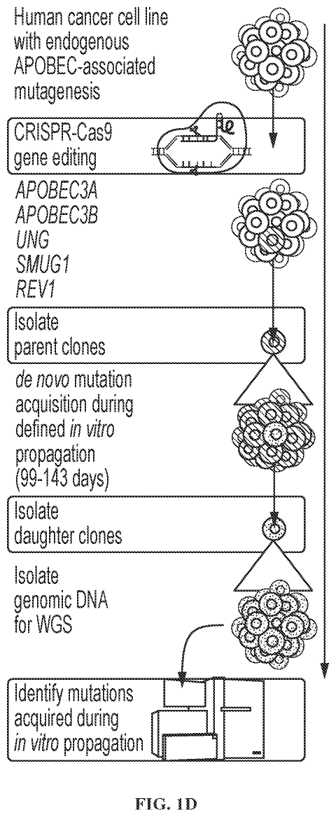

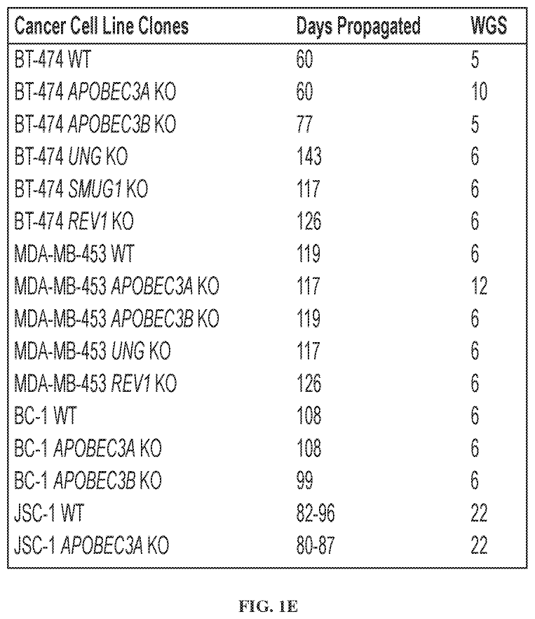

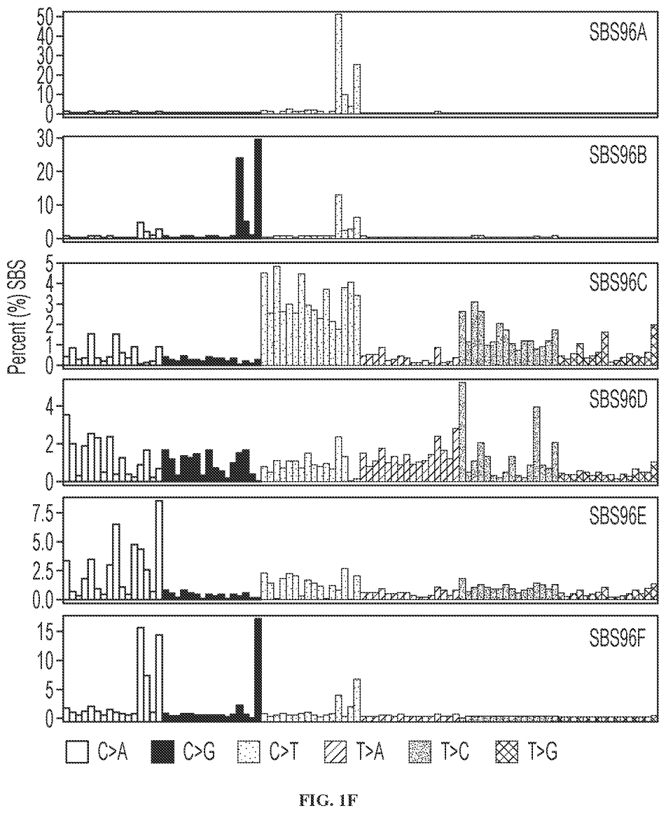

Comparison of the APOBEC3-associated mutational signatures across DNA sequences of 780 widely used human cancer cell lines and 1843 primary human cancers (FIG. 1B) revealed that the prevalence of the relevant signatures (SBS2 and SBS13) in cell lines closely resembles their prevalence across the matching types of primary cancers, whereby cancers of breast, bladder, cervix, esophageal adenocarcinoma, lung, head and neck and skin are among the most affected (Petljak et al. Cell 2019, 176(6), 1282-1294; Jarvis et al. 2018; Alexandrov et al. 2020). This was in contrast to cancers of glioma, medulloblastoma, neuroblastoma, acute myeloid leukemias, sarcomas and colorectal cancers, where these signatures are rarely found (FIG. 1B). The specific appearance of the APOBEC3-associated signatures across human cell lines suggests that these signatures do not reflect a common mutational process associated with in vitro cultivation. Instead, APOBEC3-associated signatures in cell lines most likely reflect traces of the exposures that in part occurred while the individual cell lineages were still evolving in vivo in cancer patients from which the cell lines were derived.

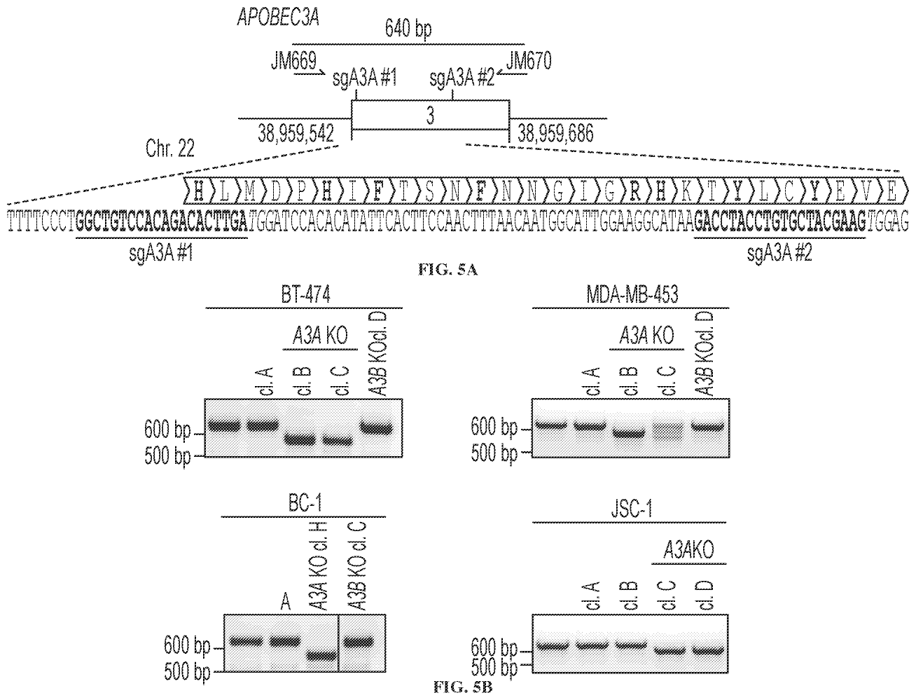

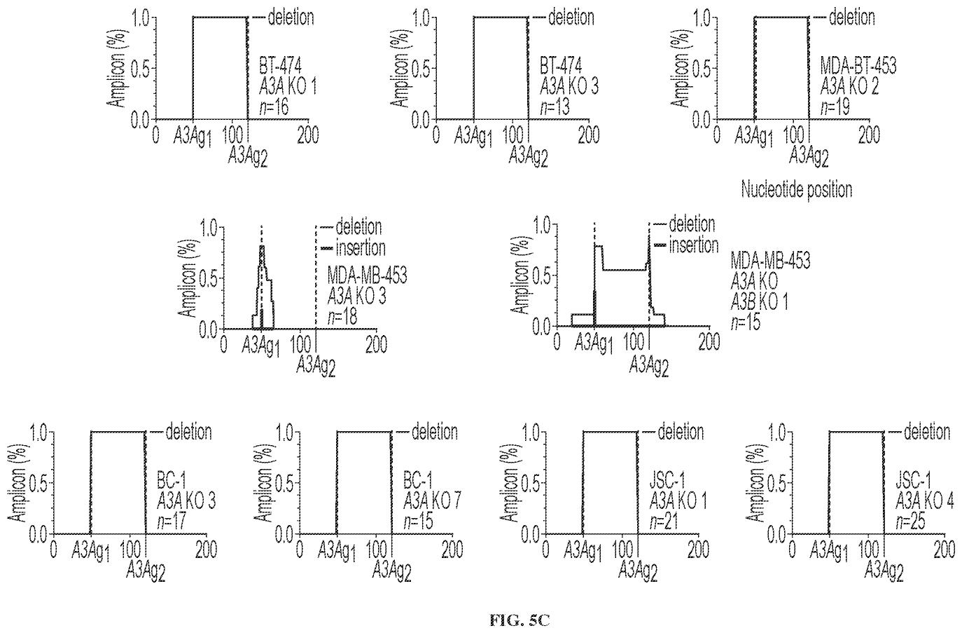

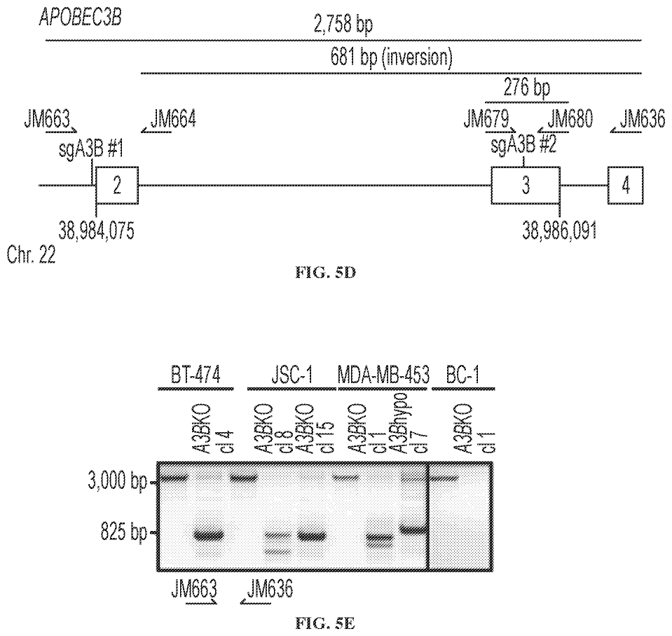

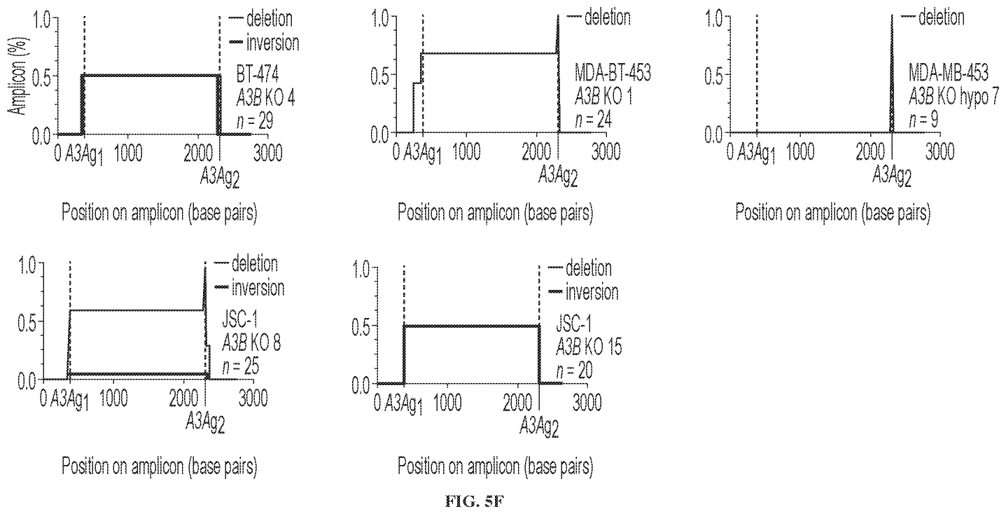

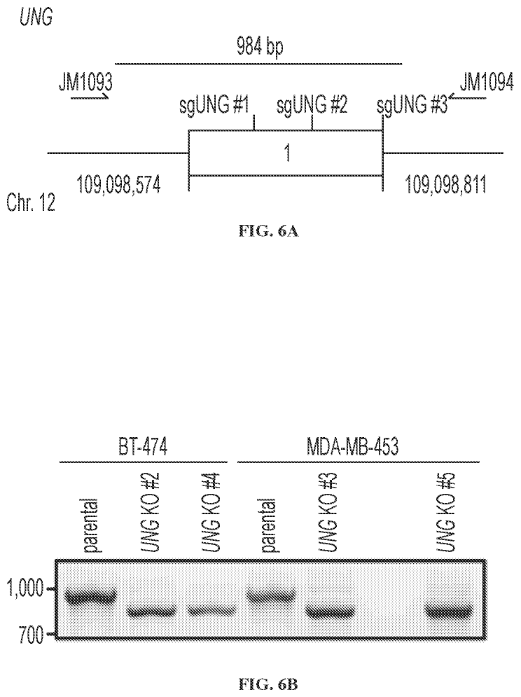

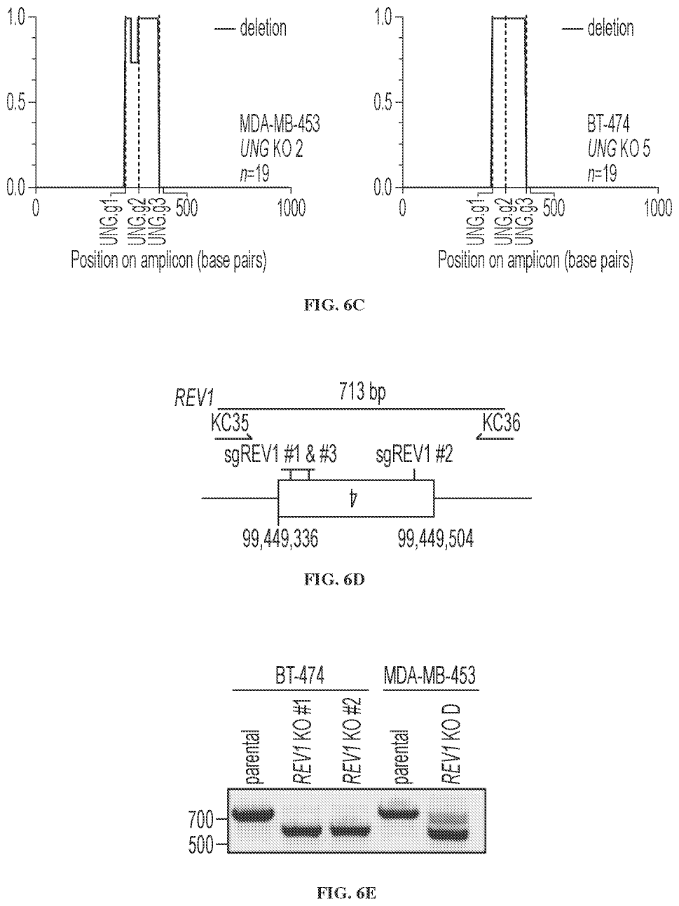

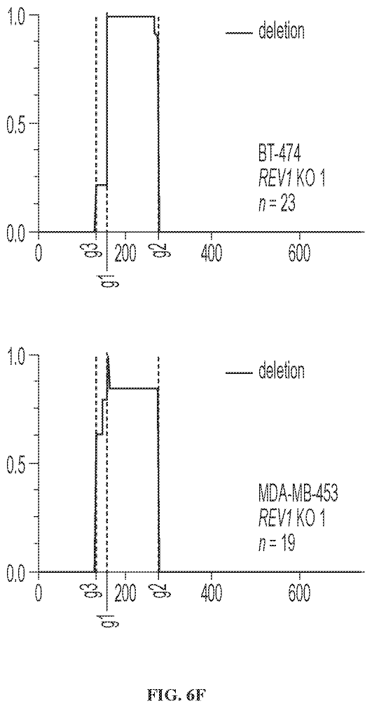

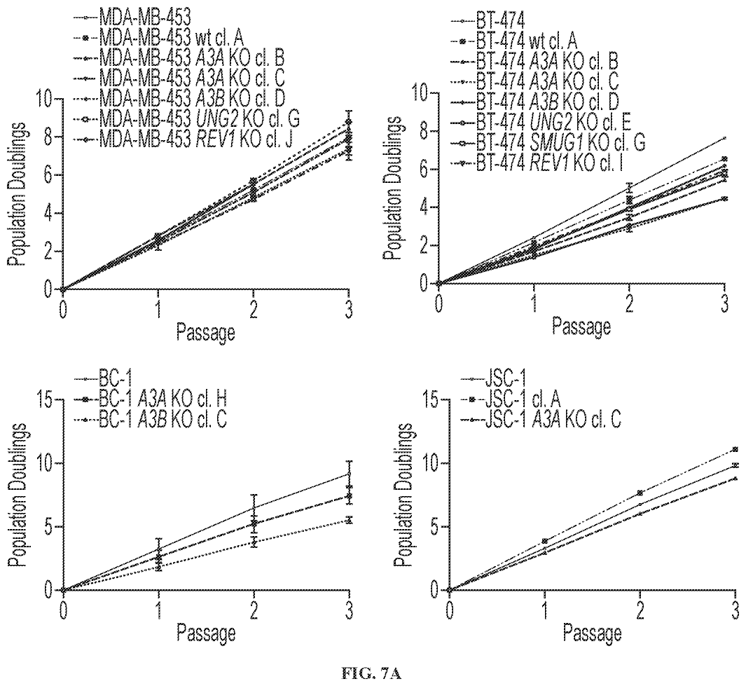

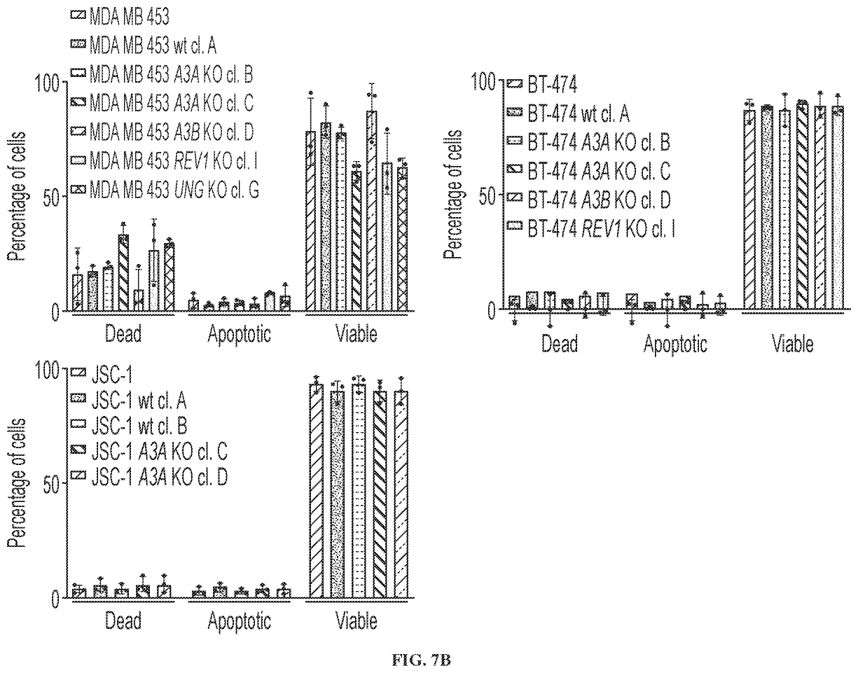

To determine the relative contributions of individual genes to generation of APOBEC3-associated signatures, a selection of candidate genes were deleted from two commonly used human breast cancer cell lines (BT-474 and MDA-MB-453), as well as two B cell lymphoma (BC-1 and JSC-1) cancer cell lines previously shown to naturally acquire clustered kataegis and unclustered APOBEC3-associated mutations over time without experimental perturbations (FIGS. 1D, 1E, 5A-5F, and 6A-6F) (Petljak et al. Cell 2019, 176(6), 1282-1294). Methods to identify de novo mutations acquired during in vitro propagation between two subcloning events were adapted to compare the mutation acquisition between the wild-type (WT) and knockout (KO) clones over controlled timeframes (FIG. 1D). In brief, constructs that express Cas9 and guide RNAs (gRNAs) against individual genes of interest were transfected into cancer cell lines by electroporation (FIGS. 1D and 1E; FIG. 5; FIG. 6). Single-cell derived WT or KO “parent” clones were subjected to long-term cultivation of 60-143 days corresponding to a timeframe over which mutation acquisition was investigated (FIGS. 1D and 1E). Following this period in culture, a further round of subcloning was carried out on the cell population from each of these parent clones, and one or more single-cell “daughter” clones were derived and shortly propagated to obtain DNA sufficient for analysis (FIG. 1D). In total, 136 individual parent and daughter clones were obtained, their individual DNAs were extracted, and ˜30× whole-genome sequence coverage was obtained for each sample. The workflow served to allow for the identification of the pre-existent mutations in bulk cell lines and individual parent cell lineages. This approach enabled identification of mutations present in daughter clones but absent from their parents, which have thus occurred over controlled in vitro periods and over equal numbers of cellular divisions between the relevant subcloning events, and have been acquired de novo over a defined period of in vitro propagation (FIGS. 1E and 7A-7B).

Examination of SBS profiles of the bulk cell lines revealed that BT-474, MDA-MB-453 and JSC-1 cell lines carried patterns of both SBS2 and SBS13, while BC-1 displayed only SBS2 signature (FIG. 1C), as reported previously (Petljak et al. Cell 2019, 176(6), 1282-1294). De novo identification of mutational signatures from 815,923 SBS discovered across all 136 clones and four bulk cell line samples revealed signatures of six ongoing mutational processes that have been operative in cell lines under analysis (FIG. 1F). Decomposition of their patterns into patterns of SBS signatures previously identified across much larger datasets from primary cancers (Petljak et al. Cell 2019, 176(6), 1282-1294) revealed that signature SBSA represents APOBEC3-associated signature SBS2 characterized by C>T mutations at TCN contexts. Signatures SBSB and SBSE were both branded by C>A, C>T and C>G mutations at TCN motifs reflecting patterns of admixed signatures SBS2 and SBS13 (Alexandrov et al. 2020), albeit individual mutation types presented at different proportions whereby SBSB is dominated by C>G and C>T mutations, while SBSE is dominated by C>A and C>T mutations. Identification of two signatures with distinct proportions of individual mutation types further corroborates the speculation that different processes that follow APOBEC3-induced DNA damage give rise to different types of cytosine mutations at TCN motifs (FIG. 1A). The identified SBSC signature reflects mixed patterns of mutational signatures SBS1, associated with 5-methylcytosine deamination, and SBS5, of unknown origin (Alexandrov et al. 2020). Both SBS1 and SBS5 signatures have been associated with processes that operate continuously throughout life across most normal and cancer cells (Alexandrov et al. 2015). SBSD reflects admixed patterns of several C>A signatures, some of which were previously attributed to oxidative stress in primary cancers and in vitro cultures (Petljak et al. 2019; Rouhani et al. 2016; Pilati et al. 2017; van Loon et al. 2010). Other identified signatures included SBS30, associated with inactivating mutations in the BER gene NTHL1 (Grolleman et al. 2019), and SBS8, SBS18, and SBS36, signatures of C>A mutations commonly attributed to oxidative stress in primary cancers and in vitro cultures (Petljak et al. 2019; Rouhani et al. 2016; Pilati et al. 2017; van Loon et al. 2010).

Next, the burdens of individual mutational signatures were quantified across the pre-existing mutations identified in parent clones and de novo acquired mutations identified in daughter clones to investigate the contributions of candidate genes to acquisition of APOBEC-associated mutations. First, the signatures identified here were deconvoluted (FIG. 1F) into their counterpart profiles that were identified previously across larger cancer datasets, which are thus more specific than their sometimes admixed patterns identified here. Second, the profiles of identified signatures identified in this way were used to estimate their contributions to mutational catalogs of each clone, across the experiments discussed below.

### Example 2: APOBEC3 Deaminases Drive and Modulate Acquisition of SBS2 and SBS13 in Human Cancer Cells

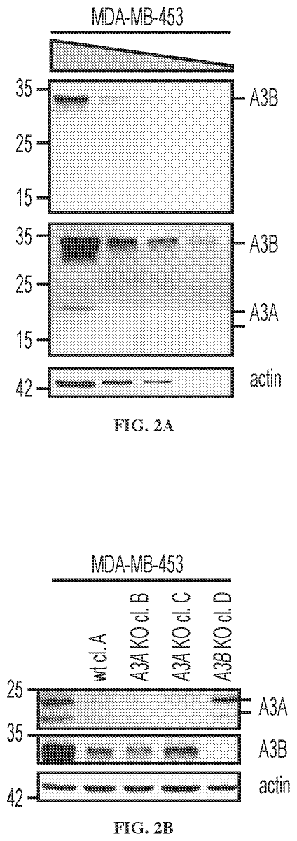

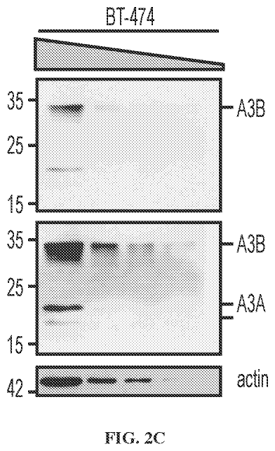

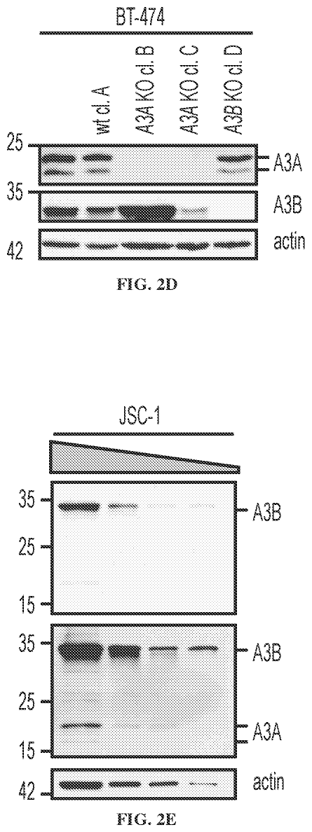

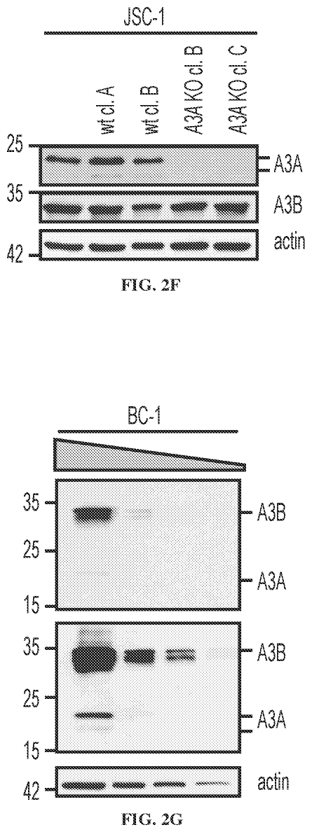

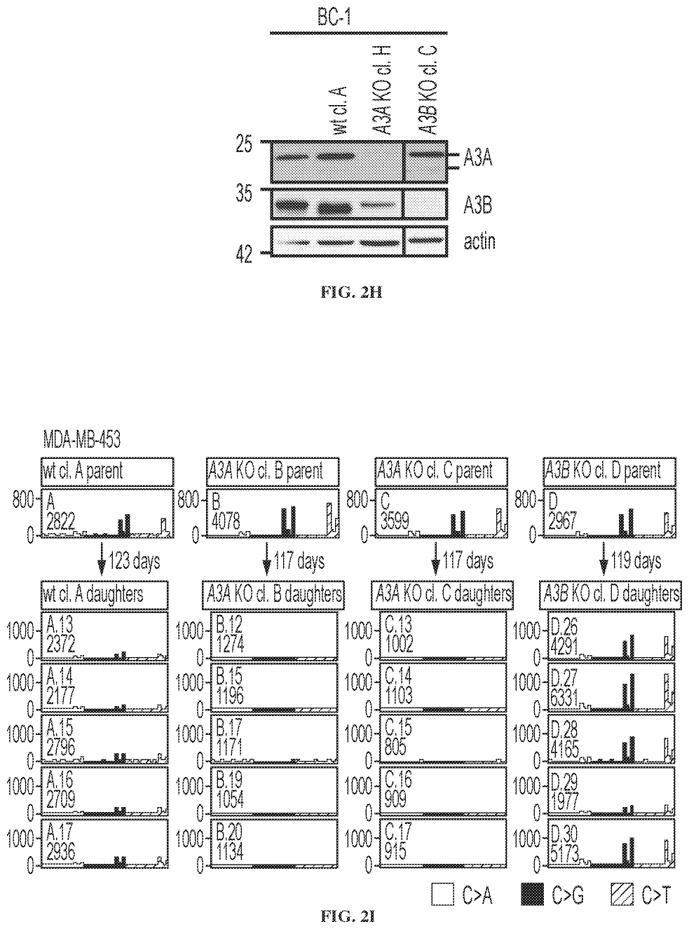

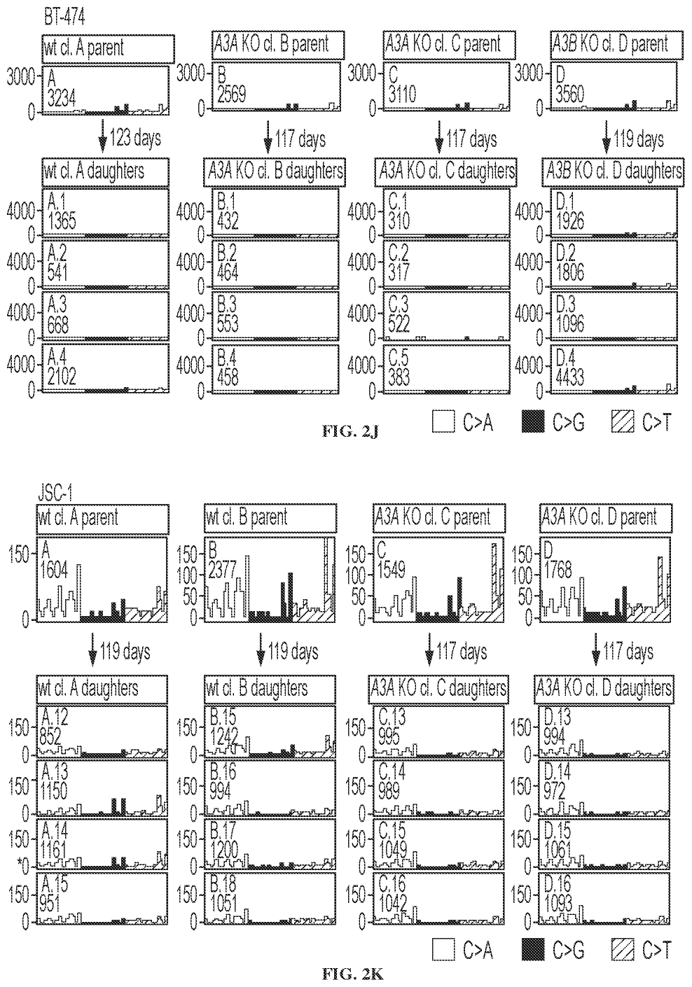

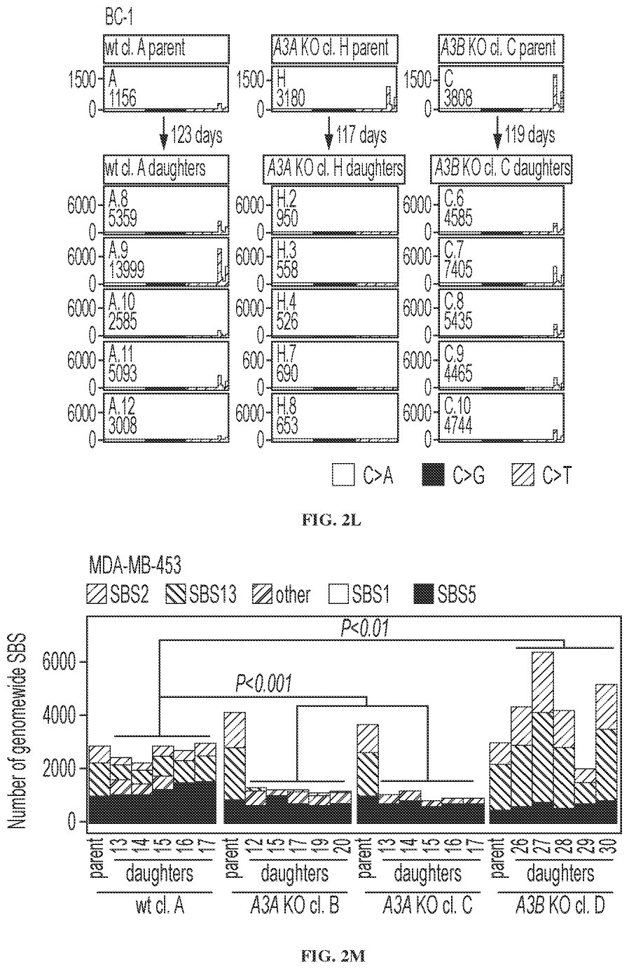

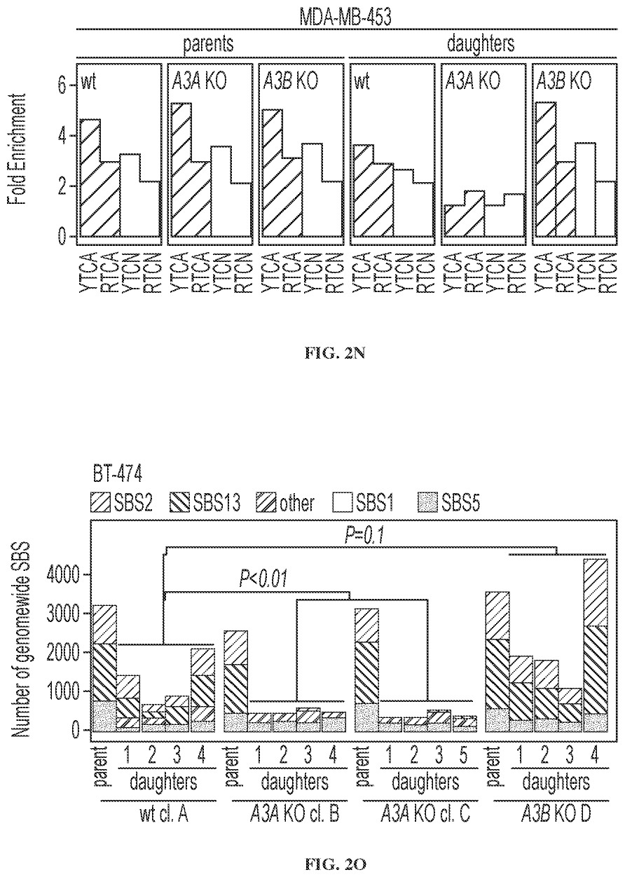

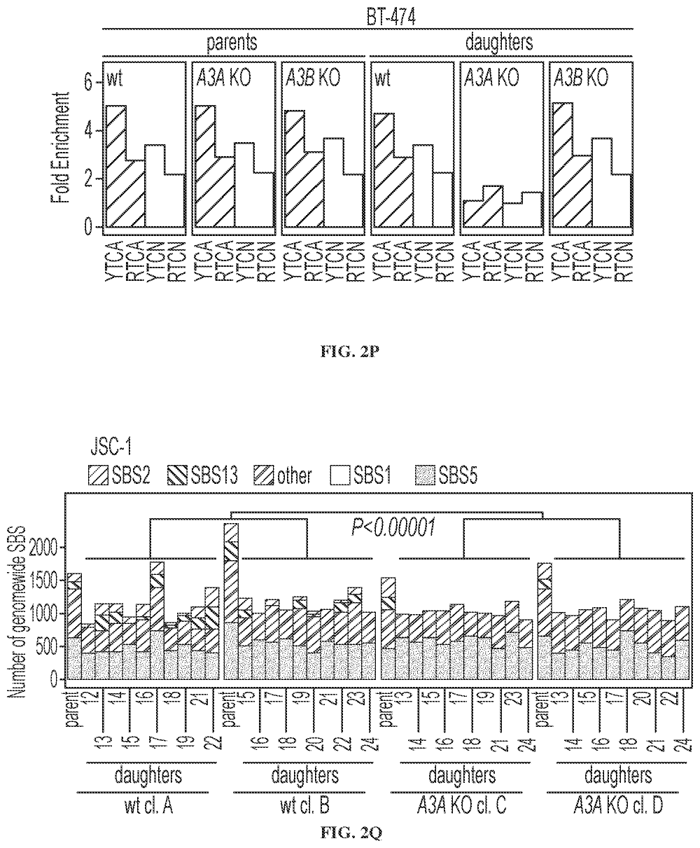

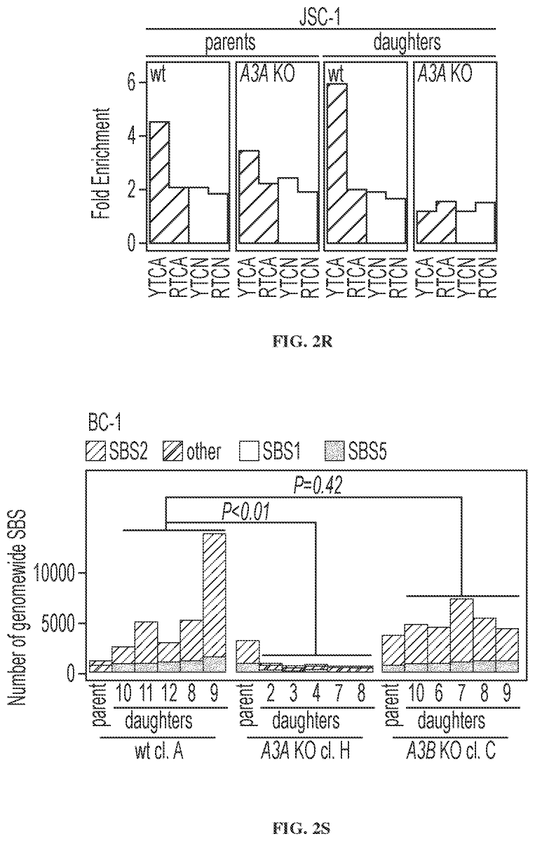

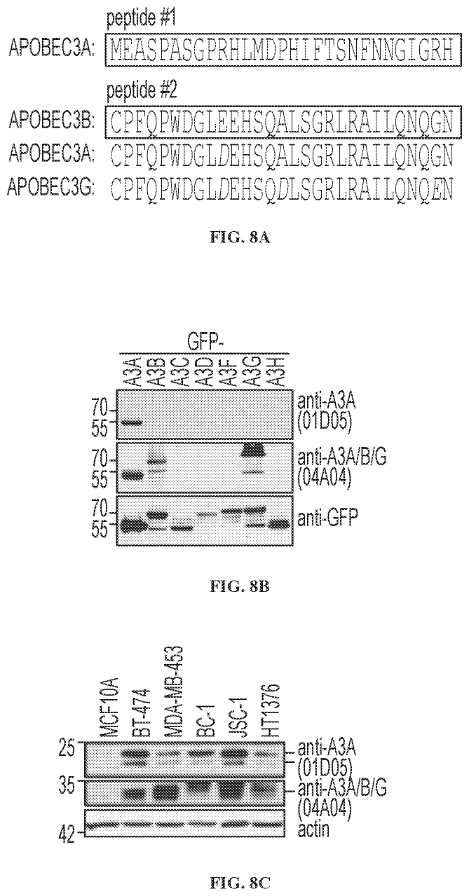

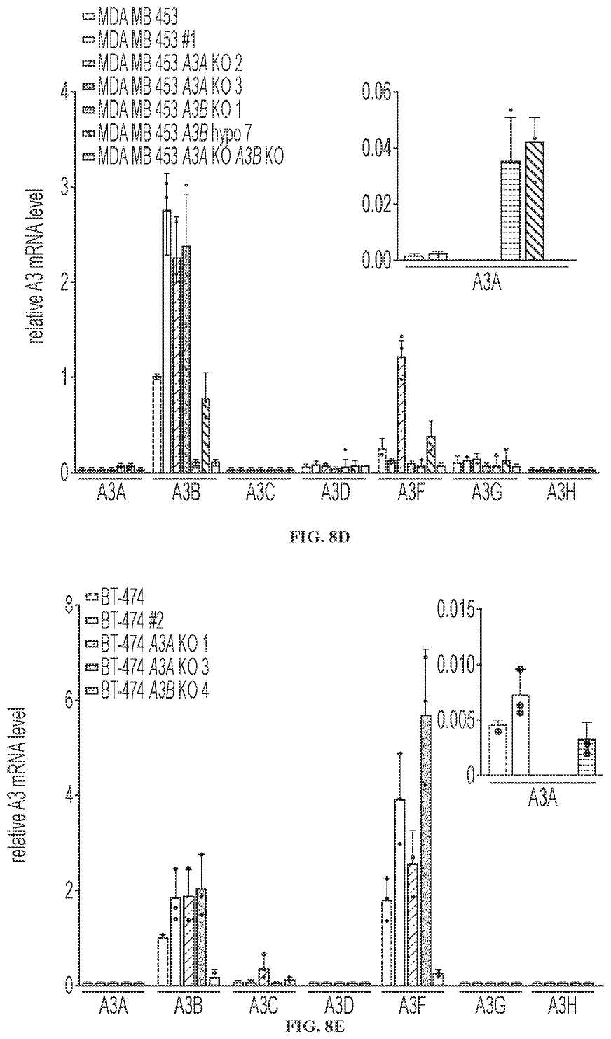

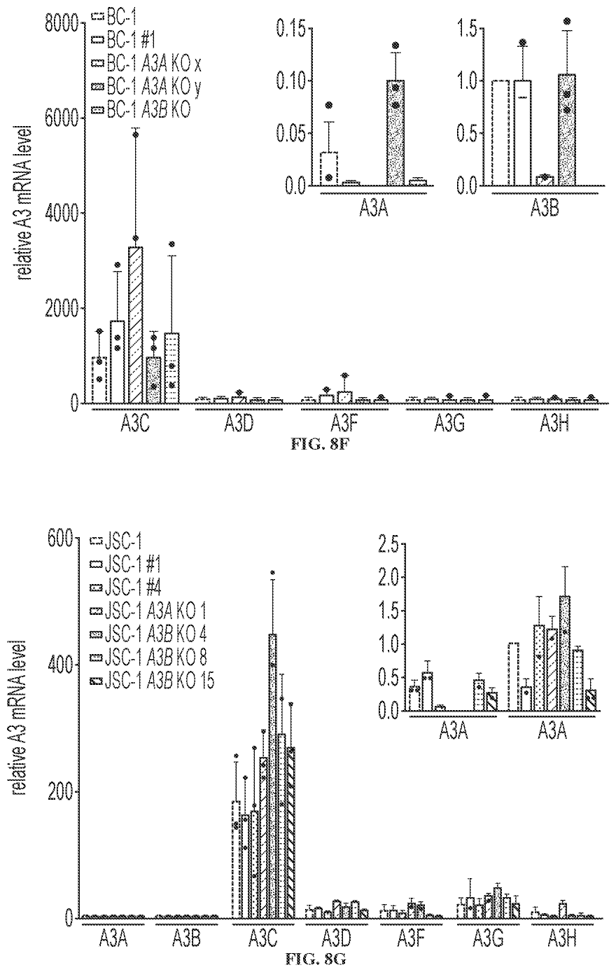

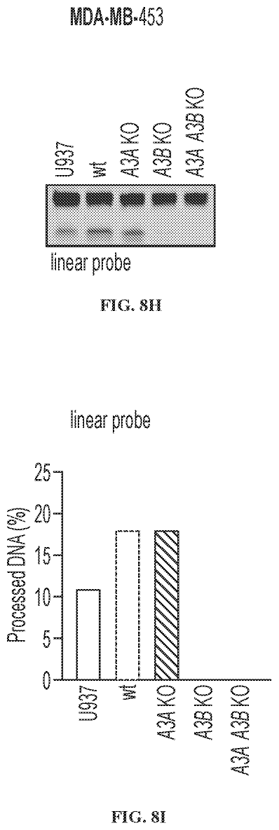

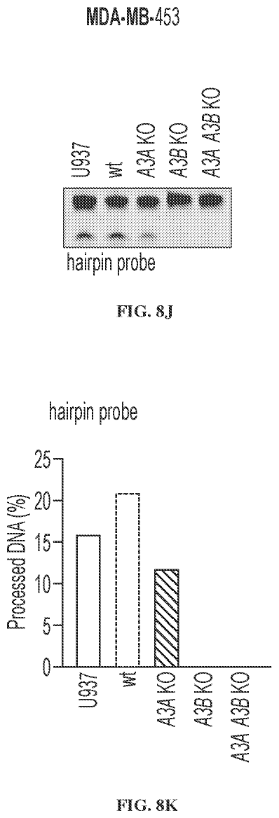

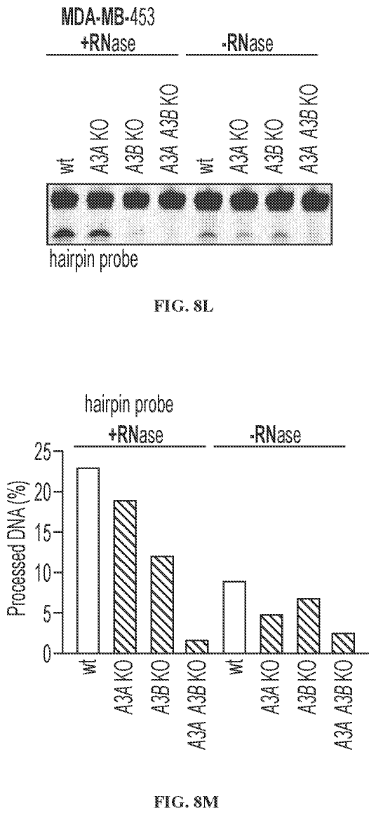

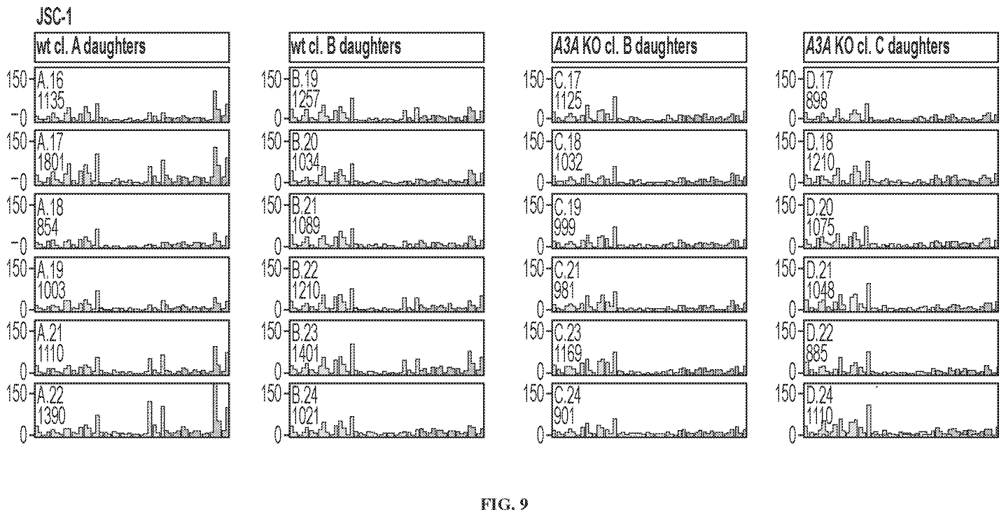

To test whether endogenous APOBEC3 activity represents an enzymatic source of cancer mutagenesis and delineate potential roles of individual APOBEC3 paralogs, APOBEC3A and APOBEC3B were deleted by CRISPR-Cas9 gene targeting from cancer cell lines with evidence of APOBEC3A and APOBEC3B expression and active APOBEC3-associated mutagenesis (FIGS. 2B, 2D, 2F, and 2H, FIGS. 5A-5F, FIGS. 2A-2F, FIGS. 8D-8G). Analysis of APOBEC3 expression by qPCR and by immunoblotting with an APOBEC3A/B/G-specific monoclonal antibody showed that APOBEC3B mRNA and protein levels were substantially elevated relative to APOBEC3A. Loss of APOBEC3A and APOBEC3B expression in targeted cell lines was verified by qPCR and immunoblotting (FIGS. 2A-H). The expression levels of non-targeted APOBEC3 paralogs fluctuated across both WT and KO clones but were not systematically affected by gene targeting (FIG. 8).

Continued generation of SBS2 and SBS13 was detectable in WT clones of breast cancer and both B cell lymphoma cell lines (FIGS. 2I-2L, FIG. 9). Unlike other mutational signatures, burdens of APOBEC3-associated mutations varied across individual daughter clones (FIGS. 2I-2L, FIG. 9). This was most prominent in BC-1 cell line, where for example, daughter A.9 from the BC-1 cell line acquired 12,598 APOBEC3-associated SBS2 and SBS13 mutations in 108 days while a daughter A.10, which was propagated in parallel and derived from the same parent clone, exhibited only 1,807 of the respective mutations. APOBEC3A and APOBEC3B expressions varied across examined WT clones, but APOBEC3B was uniformly more abundant than the minimally expressed APOBEC3A (FIGS. 8D-8G). Analysis of cytosine mutations at APOBEC3A-preferred YTCA/YTCN and APOBEC3B-preferred RTCA/RTCN sequence contexts (Y=pyrimidine base, R=purine base, N=any base) revealed enrichment of the cytosine mutations in APOBEC3A-preferred contexts (FIGS. 2N, 2P, 2R, and 2T) across wild-type clones, corresponding to the enrichment of mutations in such contexts in most cancers (Buisson et al. 2019).

Consistent with widely reported observations of upregulation of APOBEC3B in breast and other cancer types (Burns et al. Nature 2013; Burns et al. Nat. Genet. 2013; Leonard et al. 2013), all cell lines exhibited substantially elevated mRNA and protein levels of APOBEC3B relative to APOBEC3A (FIGS. 2A, 2C, 2E, and 2G; FIGS. 8A-8G). Analyses across individual wild-type clones revealed that APOBEC3A and APOBEC3B expressions varied, but APOBEC3B was uniformly more abundant than the minimally expressed APOBEC3A. In line with its elevated expression levels, APOBEC3B represented the major cytidine deaminase activity directed against linear and hairpin probes in extracts prepared from MDA-MB-453 cells (FIGS. 8H-8K). However, as reported before (Cortez et al. 2019), the presence of cellular RNA in extracts inhibited APOBEC3B activity, revealing that both APOBEC3A and APOBEC3B were enzymatically active against hairpin loop substrates in MDA-MB-453 cells (FIGS. 8L-8M). In contrast to previous reports (Cortez et al. 2019; Buisson et al. 2019), neither APOBEC3A nor APOBEC3B emerged as the dominant activity under these conditions. Deletion of each paralog elicited comparable losses in deaminase activity, and removal of both APOBEC3A and APOBEC3B was required to eliminate deaminase activity. Thus, high expression levels and deaminase activity seemingly implicate APOBEC3B as the major mutator in all cancer cell lines analyzed here, while analyses of extended sequence contexts favor a role for APOBEC3A.

Despite low expression of APOBEC3A compared to APOBEC3B in all breast and lymphoma cell lines and measurable activities from both enzymes upon DNA substrates in vitro, deletion of APOBEC3A, but not APOBEC3B, severely diminished SBS2 and SBS13 mutations in daughter clones isolated from KO parent clones (FIGS. 2I-2T, FIG. 9). For example, daughter clones isolated from a wild-type MDA-MB-453 parent clone acquired, on average, 1049±280 SBS2 and SBS13 mutations in 119 days while the daughter clones isolated from two of the MDA-MB-453 APOBEC3A KO cell lines exhibited significantly diminished numbers of the corresponding mutations over 117 days of culture (45±59 SBS; p<0.00001 one-tailed t-test; FIG. 2M). Similar results were obtained in APOBEC3A KOs of another breast (BT-474) and both B cell lymphoma cell lines (JSC-1 and BC-1), which exhibited severely diminished accumulation of SBS2 and SBS13 mutations (respectively; p<0.00001, p<0.05; p<0.001; FIGS. 2M, 2O, 2Q, and 2S; FIG. 9). Although strongly diminished, APOBEC3-associated SBS2 and SBS13 mutations were not completely eliminated in many of the APOBEC3A knockout daughter clones from BT-474, MDA-MB-453, and BC-1 cell lines, indicating that additional APOBEC3 member(s) may be generating smaller burdens of mutations in these samples. Indeed, deletion of APOBEC3A was accompanied by a shift in the enrichment of mutations from APOBEC3A-preferred YTCN to APOBEC3B-preferred RTCN sequence contexts in daughter clones (FIGS. 2N, 2P, and 2T), suggesting that APOBEC3B may also cause mutations. Taken together, these experiments implicate APOBEC3A as the main driver of SBS2 and SBS13 in breast and B cell lymphoma lines and suggest that another APOBEC3 enzyme with a likely preference for RTCN motifs, such as APOBEC3B, may also contribute.

Analysis of cytosine mutations acquired at APOBEC3A-preferred YTCN and APOBEC3B-preferred RTCN sequence contexts revealed that mutational catalogues of most WT clones were enriched in APOBEC3A-preferred YTCN contexts, in line with APOBEC3A being the major mutator (FIG. 2). Although strongly diminished, APOBEC3-associated SBS2 and SBS13 mutations were not completely eliminated in many of the APOBEC3A KO daughter clones, indicating that additional APOBEC3 members may be generating smaller burdens of mutations in these samples (FIG. 2; FIG. 9). Indeed, deletion of APOBEC3A was sometimes accompanied by a shift in the enrichment of mutations from APOBEC3A-preferred YTCN to APOBEC3B-preferred RTCN sequence contexts in daughter clones (FIGS. 2N, 2P, 2R, and 2T), suggesting that APOBEC3B may be contributing minor mutational burdens in cell lines where APOBEC3A was deleted.

While deletion of APOBEC3B did not diminish overall mutational burdens, daughter clones isolated from the APOBEC3B KO breast cancer cell lines BT-474 and MDA-MB-453 exhibited more SBS2 and SBS13 mutations on average than their WT counterparts (FIGS. 2M and 2O). This was not apparent in the BC-1 cell line, presumably due to higher levels of fluctuation in WT clones. SBS1 and SBS5 mutations, which occur independently of APOBEC3 activity and are generated continuously in cells, were summed and used to normalize mutation counts in order to control for possible differences in cell division across individual BT-474 and MDA-MB-453 APOBEC3B KO clones. This analysis revealed a significant increase in the numbers of SBS2 and SBS13 mutations in MDA-MB-453 APOBEC3B KO clones relative to WT clones (p<0.001; FIG. 2). Shorter propagation times used in the BT-474 experiments (FIG. 1E) were possibly not long enough to observe the relevant differences. Analyses of extended sequence contexts across APOBEC3B-deleted clones (FIGS. 2N, 2P, 2R, and 2T) revealed that the increased mutational burdens are usually enriched in APOBEC3A-preferred YTCN sequence contexts. Thus, while a minor mutagenic role for APOBEC3B in the examined cell lines cannot be excluded, it is possible that APOBEC3B limits APOBEC3A-driven episodic mutagenesis by an unknown mechanism. The increase in mutations in the MDA-MB-453 cell line was reminiscent of the higher APOBEC3-associated mutational burdens observed in breast cancers that develop in carriers of a common germline deletion polymorphism that effectively deletes APOBEC3B (Nik-Zainal et al. 2014). The mechanisms underlying these observations remain unknown. Burdens of SBS5 occasionally varied in clones from the MDA-MB-453 and BC-1 cell lines, albeit not as substantially as burdens of SBS2 and SBS13 (FIGS. 2M and 2S). SBS30, SBS8, SBS18, and SBS36 contributed small numbers of mutations compared to other signatures. The sums of mutations attributed to these signatures were thus represented together (“other”) and fluctuated across individual clones due to mutational burdens that were underpowered for accurate quantification (FIGS. 2M, 2O, 2Q, 2S). Taken together, these experiments implicate APOBEC3A as a main driver of SBS2 and SBS13 in breast and B cell lymphoma lines and suggest that another APOBEC3 enzyme with a likely preference for RTCN motifs, such as APOBEC3B, may also contribute mutations.

### Example 3: Base-Excision Repair Plays a Critical Role in Generation of APOBEC3 Mutations in Cancer

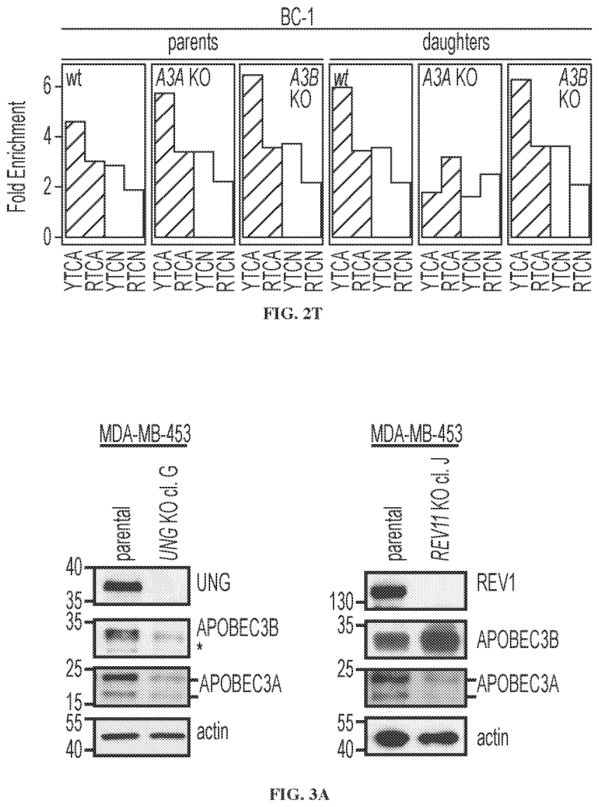

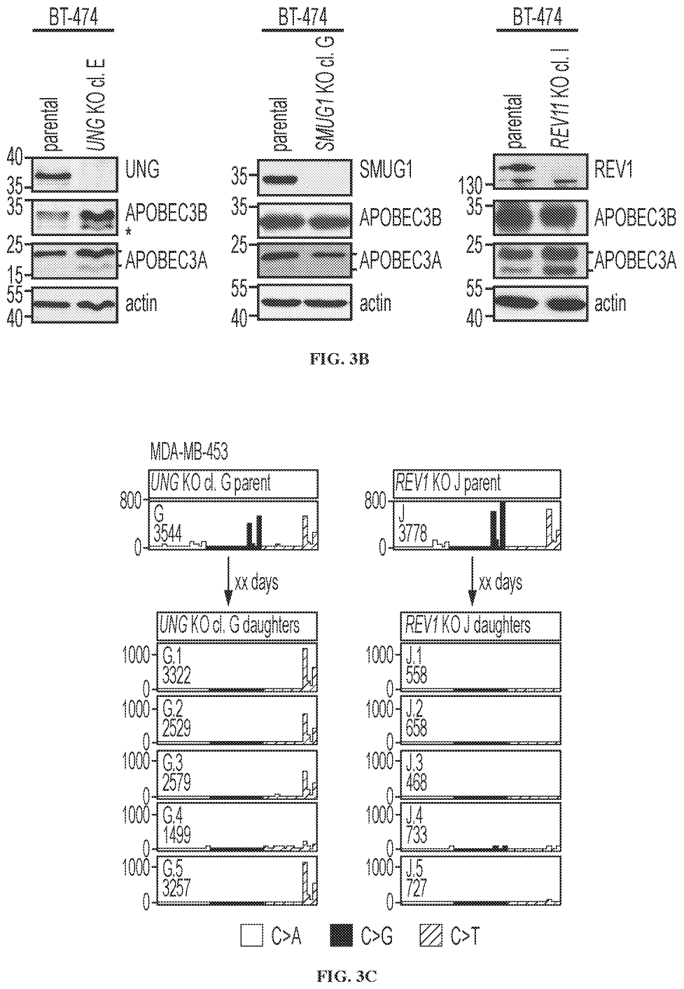

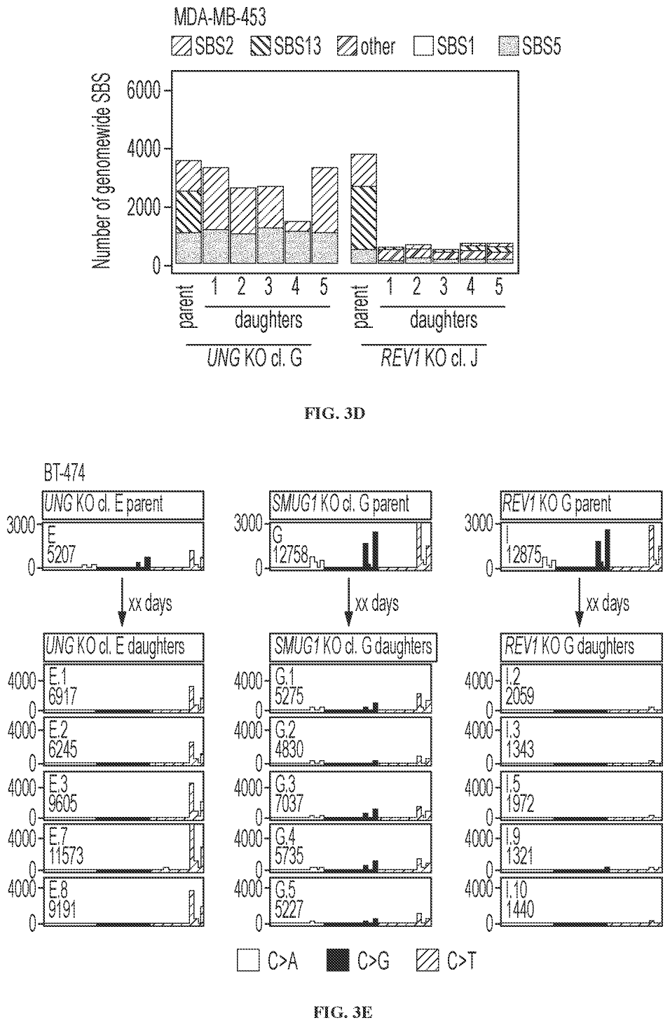

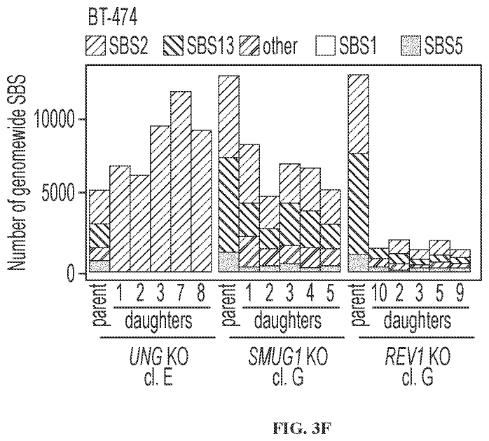

Most cancers and cell lines with mutational signatures of APOBEC3 deaminases exhibit both SBS2 and SBS13 signatures, albeit at different relative proportions (FIG. 1B) (Alexandrov et al. 2020; Alexandrov et al. 2013; Petljak et al. 2019). The presence of SBS2 in the absence of SBS13 is shown here for the first time in a BC-1 lymphoma cell line (FIG. 1C; FIG. 2S). To directly assess the impact of BER on the generation of SBS2 and SBS13 in cancer cells (FIG. 1A), the uracil glycosylases UNG and SMUG1 were deleted in BT-474 and MDA-MB-453 cells by CRISPR/Cas9 editing. Successful gene targeting was confirmed by PCR and Sanger sequencing and loss of expression was verified by immunoblotting (FIG. 6; FIGS. 3A and 3B).

In sharp contrast to WT clones from MDA-MB-453 and BT-474 cell lines, which exhibited both SBS2 and SBS13, daughters isolated from the UNG KO clones exhibited exclusively SBS2 mutations (FIGS. 3C-3F). This confirms that generation of transversion mutations in SBS13 depends on UNG-dependent uracil excision following APOBEC3-mediated cytosine deamination (model in FIG. 1A). In MDA-MB-453, where UNG KO clones were propagated to a similar number of days as WT clones (respectively, 117 and 119 days), UNG deletion did not elicit a substantial impact on the overall burden of SBS2 and SBS13 mutations (P=0.07, Mann-Whitney test; FIGS. 2I, 2M, 3C, and 3D). Thus, most of the unexcised uracils generated by APOBEC3A base editing appear to be converted into C>T mutations by UNG-independent mechanisms including DNA replication (FIG. 1A). Deletion of the nuclear uracil DNA glycosylase SMUG1, which can occasionally substitute for UNG (Nilsen et al. 2001), did not substantially affect overall cytosine mutational burden or the acquisition of C>A or C>G transversions in BT-474 and MDA-MB-453 cancer cell lines (FIGS. 3E and 3F). These results indicate that SMUG1 is dispensable for the generation of SBS13. The observed dependency on UNG for the processing of APOBEC3A-generated uracils may derive from its ability to process both single-stranded and double-stranded DNA (dsDNA), while SMUG1 activity is essentially specific to dsDNA (Doseth et al. 2012).

Following uracil excision, replication across abasic sites by translesion synthesis (TLS) polymerases has been speculated to give rise to C>A and C>G transversions, as well as a portion of C>T mutations, based on models of activation-induced cytidine deaminase (AID) APOBEC family member during immunoglobulin gene somatic hypermutation (Masuda et al. 2009; Sale et al. 2012). Specifically, Rev1 was proposed to form a scaffold for components of TLS upon AID-mediated somatic hypermutation mediated by the AID APOBEC family member and to thus play a critical role in generation of a broad range of TLS-associated mutations (Simpson 2003). To assess the contribution of TLS to generation of SBS2 and SBS13, REV1 was targeted by CRISPR/Cas9 editing in breast cancer cell lines, and loss of expression was verified by immunoblotting (FIGS. 6D-6F). Consistent with the role of REV1 during AID-mediated somatic hypermutation (Simpson 2003; Ross and Sale 2006), both transition and transversion mutations were severely diminished in daughter clones isolated from REV1 KO parent clones (FIGS. 3C-3F). In MDA-MB-453 cells, this led to almost a 6-fold decrease in SBS2 and SBS13 in REV1 KO clones compared to WT clones (p<0.0001), while in BT-474 to more than a 4-fold decrease when REV1 KO clones were compared to UNG/SMUG1 KO clones that were propagated for a similar number of days (both p<0.0001) (FIGS. 3C and 3D). These results suggest that REV1 and TLS play a critical role in the generation of both SBS2 and SBS13, but not all SBS2 and SBS13 mutations are dependent on Rev1. Substantial depletion of SBS2 signature mutations in REV1, but not UNG KOs, suggests that REV1 may have a key role in generation of C>T mutations that is not associated with BER-associated TLS.

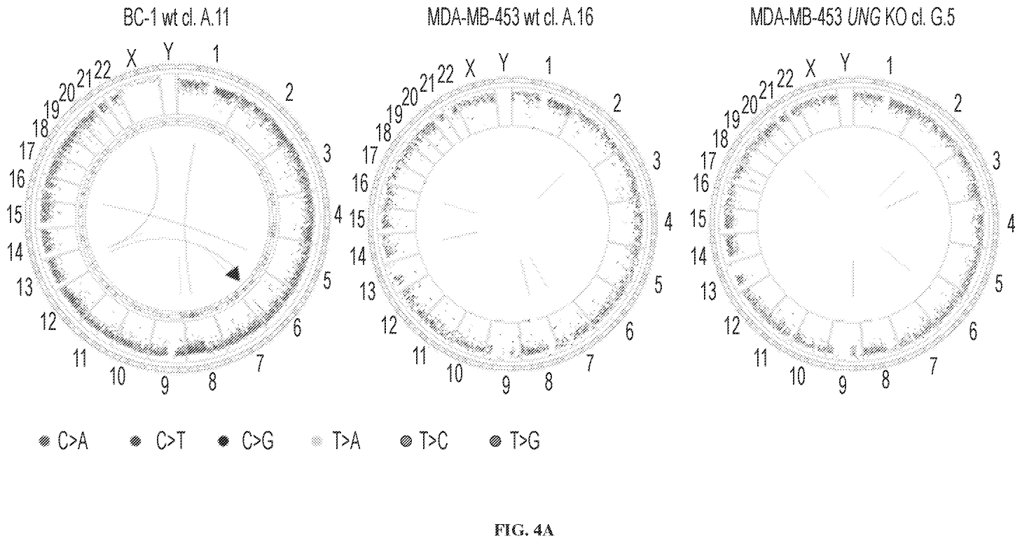

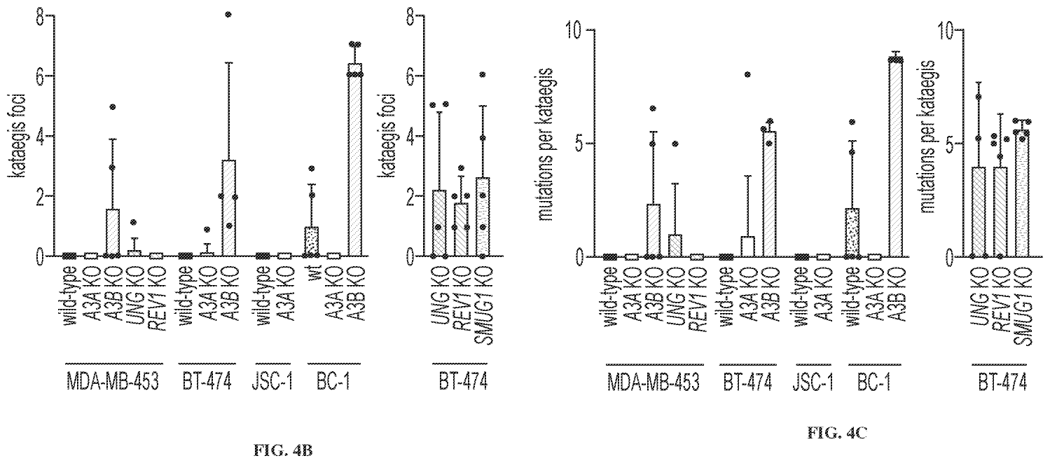

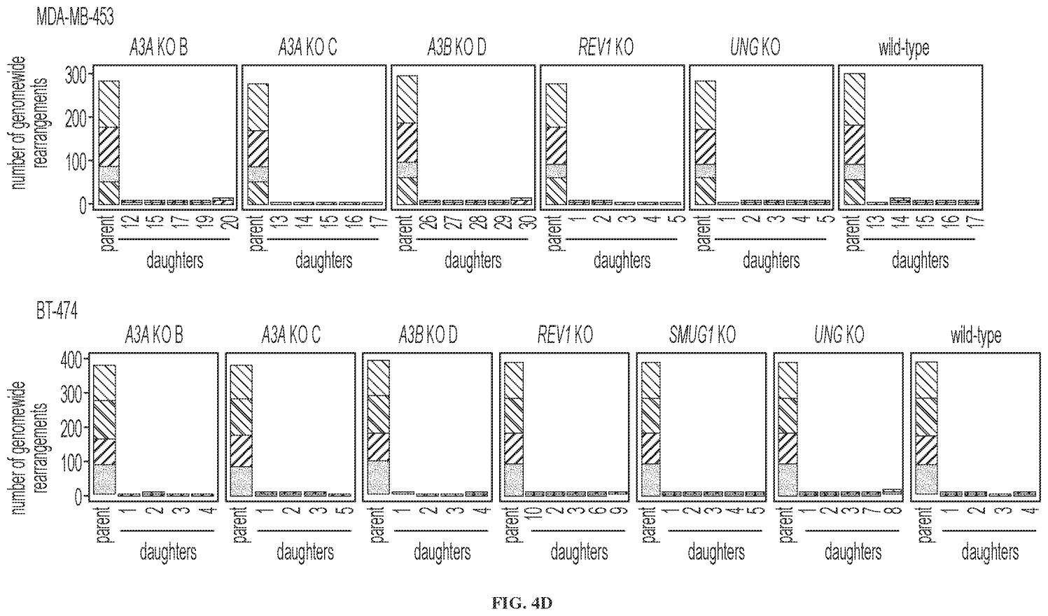

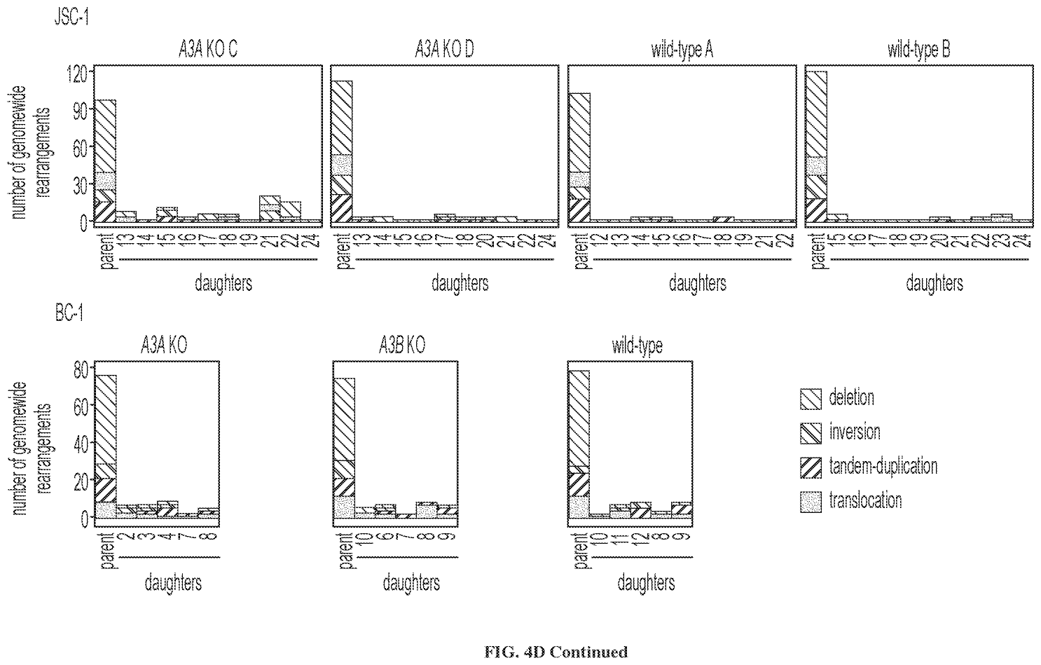

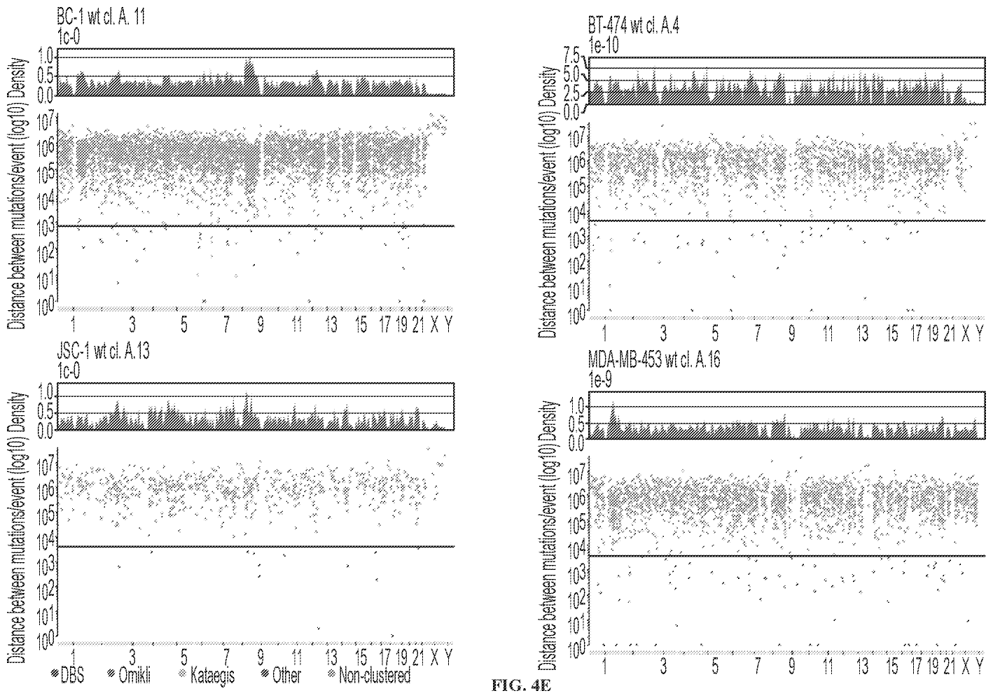

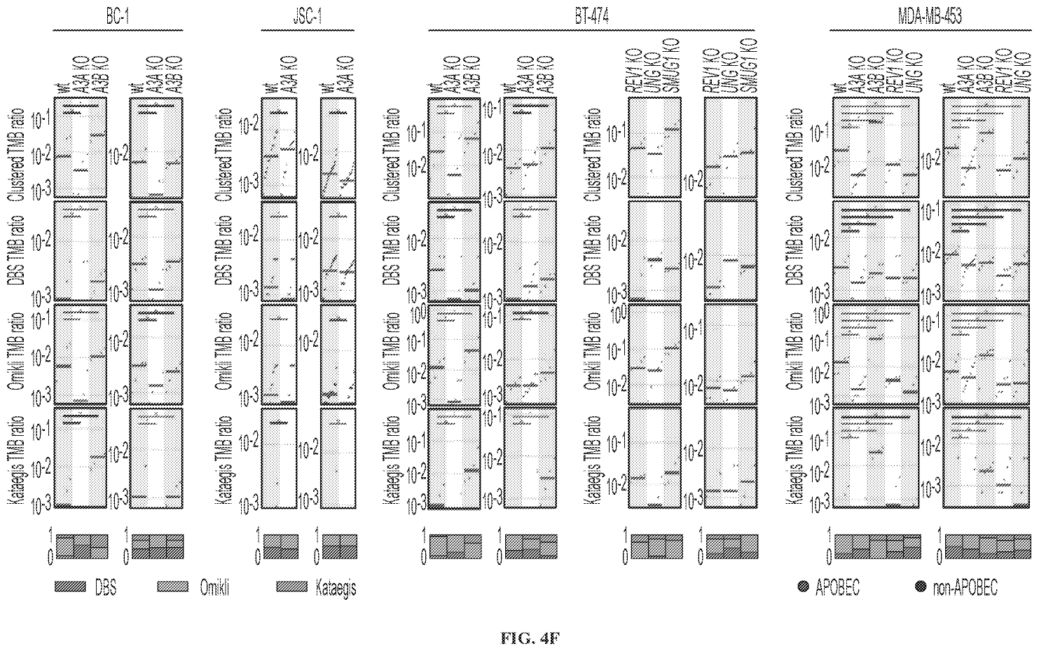

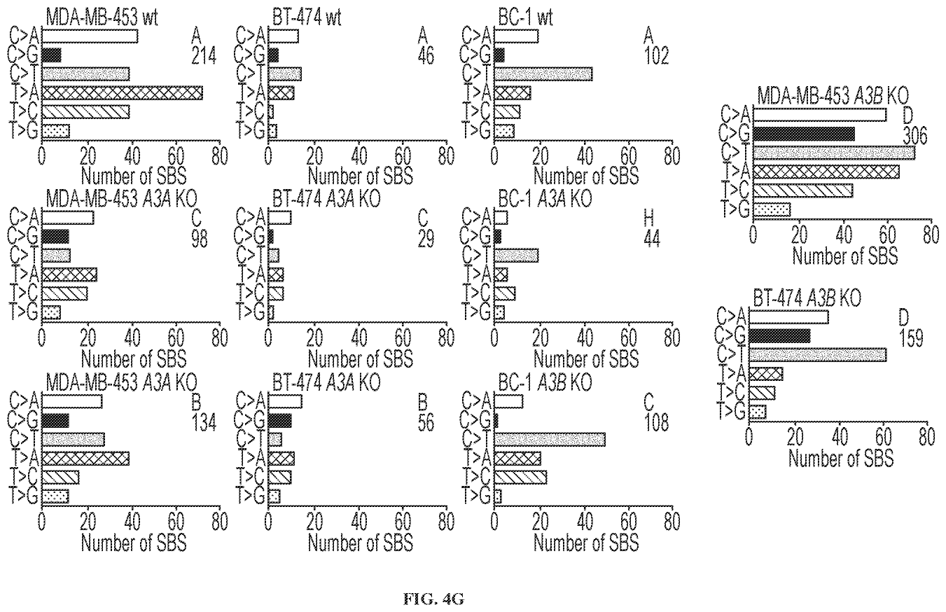

Furthermore, the data suggests that in the absence of REV1, alternative, less mutagenic pathways, may be used to navigate the lesion. One such possibility is recombination-mediated bypass, which has previously been proposed to act downstream of AID (Simpson 2003). While such pathways were proposed to come at a cost of the increased genomic instability, an increase in rearrangements was not observed (FIGS. 4A and 4B). Alternatively, however, it is possible that cells with extensive chromosomal aberration were negatively selected in the population of parent cells. Depletion of SBS2 and SBS13 signature mutations in the REV1 KOs appears to occur independently of disturbed growth or APOBEC3A depletion (FIGS. 3A and 3B; FIG. 8). Nevertheless, the possibility that APOBEC3A mutagenic episodes were synthetically lethal or selected against in REV1 KO cells cannot be excluded.

Unlike SBS1, mutational burdens attributed to SBS5 were significantly depleted in REV1 knockout cells of MDA-MB-453 cell lines (p=4.0×10-3, Mann-Whitney test). SBS5 has been attributed to an unknown process that is continuously operative across all tissues (Alexandrov et al. 2015; Kim et al. 2016), and its increased burdens in bladder cancers have been associated with mutations in the ERCC2 gene encoding a DNA helicase that plays a central role in the NER pathway (Kim et al. 2016). The data suggests that REV1 may play a critical part in the underlying mutational process.

### Example 4: APOBEC3 Deaminases Drive Acquisition of Kataegis and Omikli Mutations in Human Cancer Cells

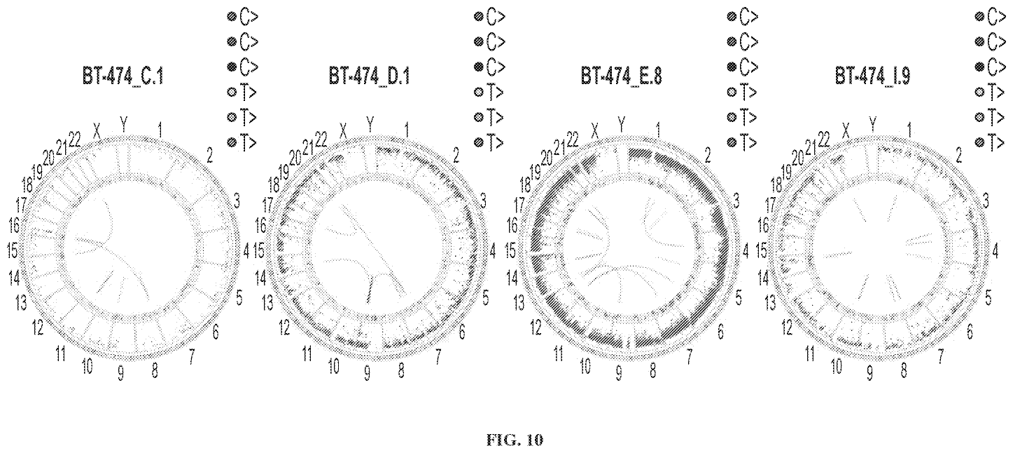

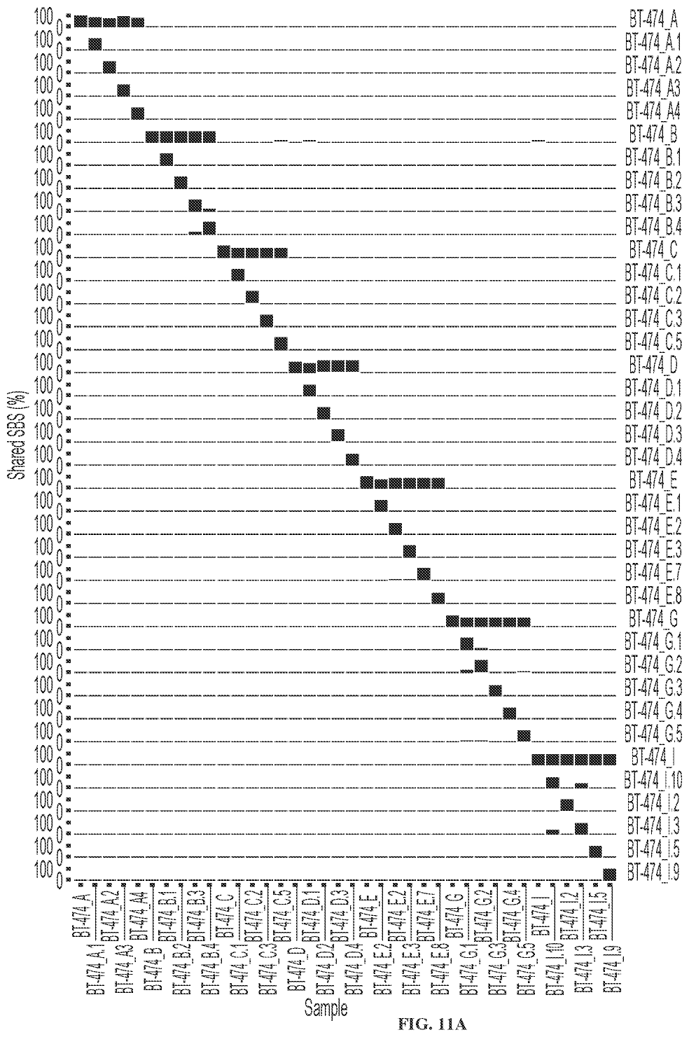

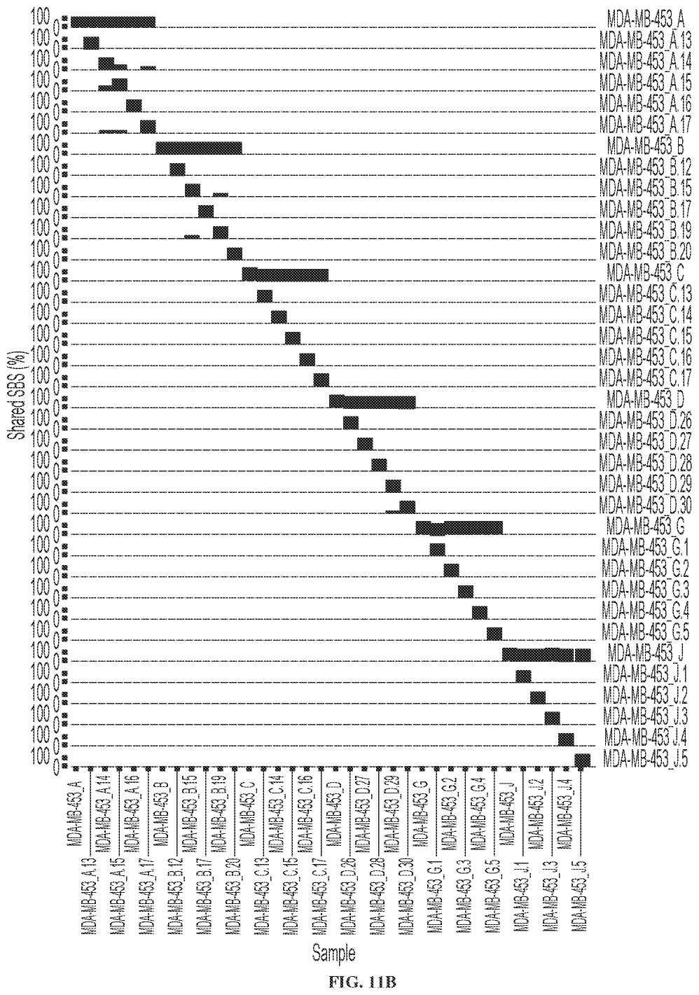

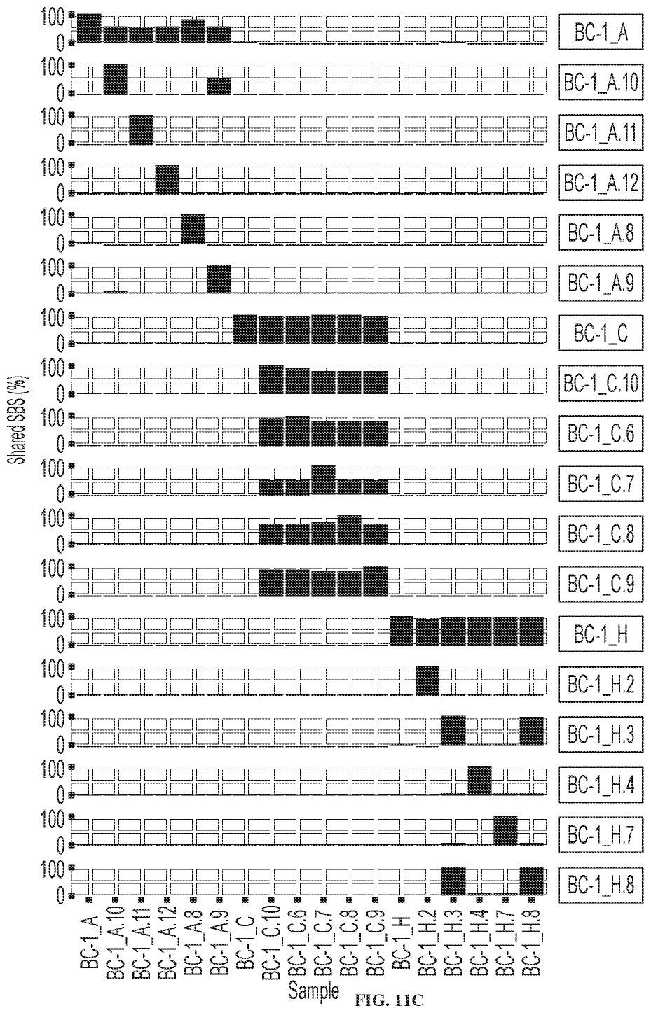

Most APOBEC3-associated mutations in examined cell lines were dispersed throughout the genome (FIG. 4A; FIG. 10). The ongoing acquisition of hypermutation kataegis foci, characterized by densely clustered cytosine mutations occurring at TCN motifs in cis with respect to each other, was previously reported in cell lines under analysis (Petljak et al. 2019). All cell lines acquired additional smaller numbers of clustered mutations, which commonly presented at the APOBEC3-associated cytosine mutations in TCN sequence contexts, including kataegis foci of densely clustered SBS mutations, omikli clusters of more sparsely distributed SBS mutations, and doublet base substitutions (DBS) (FIG. 4E). Kataegis was observed in WT BC-1 cells, albeit rarely (FIGS. 4A-4C; FIG. 10). Deletion of APOBEC3A, but not APOBEC3B, eliminated kataegis foci and omikli clusters from the BC-1, MDA-MB-453, and BT-474 cell lines (FIG. 4F). Indeed, consistent with the increased burden of genome-wide SBS2 and SBS13 observed in APOBEC3B-deleted clones from MDA-MB-453, there was an elevated number of APOBEC3-like kataegis foci in APOBEC3B knockout clones from all cell lines, and APOBEC3-like omikli was increased in APOBEC3B knockout clones from the breast cancer cell lines (FIG. 4F). Neither APOBEC3A nor APOBEC3B were required for generation of kataegis foci and omikli, as both were observed in BT-474 APOBEC3A KO daughters (FIG. 4B). Taken together these data indicate that APOBEC3A is the main driver of APOBEC-like kataegis and omikli, but suggest that additional mutators, such as APOBEC3B, may play a minor role as previously proposed (Maciejowski et al. 2020).

In line with an increased burden of genome-wide SBS2 and SBS13 observed in APOBEC3B-deleted clones, the highest number of kataegis foci were observed in APOBEC3B KO clones from all cell lines (FIG. 4A-4C). Thus, the mechanisms underlying misregulation of APOBEC3 that leads to generation of genome-wide and clustered APOBEC3 mutations are at least in part the same. Taken together these data indicate that APOBEC3A is the predominant driver of kataegis, while APOBEC3B may modulate kataegis acquisition.

Unexpectedly, loss of APOBEC3A also caused a reduction in clustered mutations occurring outside of APOBEC3-like sequence contexts in BC-1 and MDA-MB-453 cells, while deletion of APOBEC3B led to their modest increase in breast cancer cell lines (FIGS. 4F and 4G). These SBS primarily consisted of C>T transitions, consistent with the possibility that they may derive, in part, from non-canonical APOBEC3A base editing at exposed regions of ssDNA.

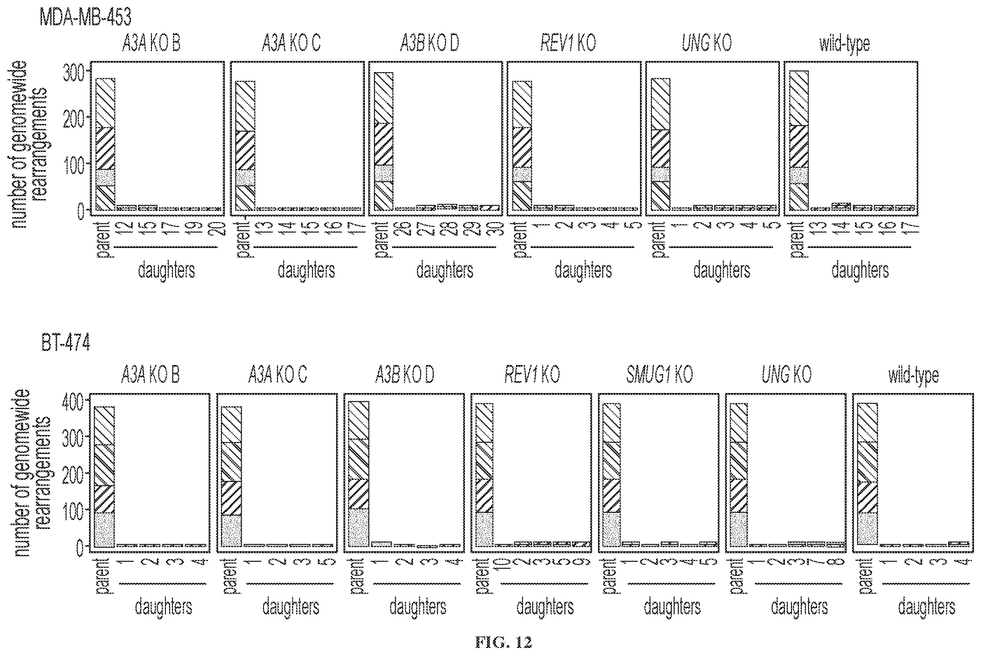

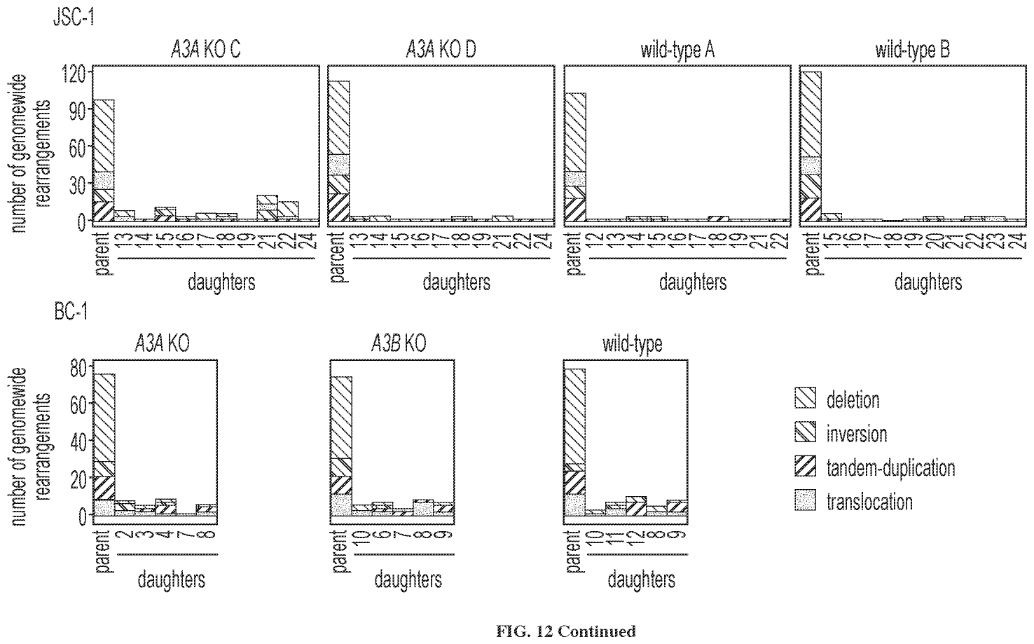

Kataegis foci often co-localize with rearrangements in primary cancers, a phenomenon in part attributed to APOBEC3 attacks on ssDNA exposed during the resection phase of homologous recombination-mediated double-strand break DNA repair (Taylor et al. 2013; Nik-Zainal et al. 2012). A separate explanation proposes that APOBEC3-induced deamination may precede the dsDNA breaks (Taylor et al. 2013), if ssDNA breaks generated upon UNG-mediated uracil excision represent the initiating lesions for formation of subsequent dsDNA breaks. However, kataegis did not depend on UNG in MDA-MB-453 and BT-474 cell lines (FIGS. 4B and 4C). Additionally, there were several examples of kataegis foci that appeared to occur independently of any proximal rearrangements in cell line clones (FIG. 4A; FIG. 10). These data suggest that mechanisms other than DNA double-strand break repair may underlie the generation of kataegis. However, the possibility that initiating DNA double strand breaks were successfully repaired cannot be excluded as cell lineages harboring chromosome rearrangements may have been selected against during in vitro propagation (FIG. 4D; FIG. 12). Finally, in line with REV1 contributing to a broader spectrum of SBS mutations (FIGS. 3C-3F), including non-clustered signatures SBS5 and APOBEC-associated SBS2 and SBS13, deletion of REV1 in MDA-MB-453 cells resulted in reduced mutational burdens of clustered mutations occurring both within and outside of the APOBEC3-like sequence contexts.

### Discussion

The present disclosure provides the first direct evidence that cytidine deaminases represent potent mutators in human cancer cells. The data establish APOBEC3A as the main driver of highly prevalent genome-wide and clustered kataegis APOBEC3-associated mutational signatures in breast and B cell lymphoma cancer cells. APOBEC3-associated mutational signatures are enriched at YTCN sequence contexts in the majority of individual human cancers and cancer types (Chan et al. 2015; Burns et al. Nature 2013; Burns et al. Nat. Genet. 2013). The finding described herein that APOBEC3A accounts for most APOBEC-associated mutations at YTCN sequence contexts in human cancer cells strongly indicates that APOBEC3A drives acquisition of the large majority of all APOBEC-associated mutations observed in cancer genomes. All the cancer cell lines analyzed in this study, where APOBEC3A is the predominant driver of the relevant mutations, possess high levels of APOBEC3B expression relative to APOBEC3A, an observation that was previously used to nominate APOBEC3B as the major mutator in cancer (Burns et al. Nature 2013; Burns et al. Nat. Genet. 2013; Leonard et al. 2013). Furthermore, despite APOBEC3A being the predominant mutator, activities of APOBEC3A and APOBEC3B were similar in in vitro deamination assays that have commonly been used as substitute readouts of mutagenesis by individual enzymes (Burns et al. Nature 2013; Burns et al. Nat. Genet. 2013). Thus, the data show that increased expression and deamination activities of individual APOBEC members may not always translate into active mutagenesis. These findings caution against the widespread use of such readouts as sole substitute measures of active mutagenesis by APOBEC3 deaminases, which resulted in distinct predictions regarding APOBEC members as predominant mutators in cancer (Burns et al. Nature 2013; Burns et al. Nat. Genet. 2013; Cortez et al. 2019; Jalili et al. 2020). The direct measurements of mutagenic activities of APOBEC3A and APOBEC3B enzymes in human cancer cell line genomes used here represent the strongest available support that mutagenesis by APOBEC3A, and not APOBEC3B, represents the major source of some of the most prevalent mutational signatures in human cancer. Recent work, largely based on correlations between individual APOBEC3 expression levels and deamination activities, has implicated distinct APOBEC3 members as drivers of targeted therapy resistance in lung cancers (Mayekar et al. 2020; Isozaki et al. 2021). The results described herein call for the use of more direct measures of APOBEC3 activity to delineate the role of individual APOBEC3 enzymes in cancer genome evolution.

Finally, these data implicate UNG and REV1, and thus BER, in the generation of APOBEC3-induced non-clustered signatures SBS2 and SBS13, as well as clustered kataegis and omikli events in cancer cell genomes. Experimental confirmation of APOBEC3 deaminases as mutators in human cancer cells and identification of APOBEC3A as the main generator of widespread mutations in cancer marks a critical advance in pursuing therapeutic interventions based on modulating the generation of the associated SBS signatures and in investigating the origins of APOBEC3-associated mutations in cancer. These data show that modulation of mutagenic activities by APOBEC3A offers avenues for therapeutic interventions.

### Methods

Cell Culture: MDA-MB-453, BT-474, JSC-1, and BC-1 cancer cell lines were acquired from the cryopreserved aliquots of 1,001 cell lines, extensively characterized as part of the Genomics of Drug Sensitivity in Cancer (GDSC) (Iorio et al. 2016; Garnett et al. 2012) and COSMIC Cell Line projects (Petljak et al. 2019; Forbes et al. 2017). Cell lines were genotyped previously by SNP and STR profiling, as part of the COSMIC Cell Line Project (cancer.sanger.ac.uk/cell_lines) and individual clones obtained here were Fluidigm genotyped to ensure that their accurate identities. MCF10A cells were from Maria Jasin (MSKCC).

All cell lines were mycoplasma negative and fingerprinted by single nucleotide polymorphism (SNP) and short tandem repeat (STR) profiling at the MSKCC Antibody and Bioresource Core. MDA-MB-453 cells were grown in DMEM:F12 medium supplemented with 10% fetal bovine serum (FBS) and 100 U/mL penicillin-streptomycin. BC-1, BT-474, and JSC-1 cells were grown in RPMI medium supplemented with 10% FBS, 1% penicillin-streptomycin, 1% sodium pyruvate, and 1% glucose. MCF10A cells were cultured in 1:1 mixture of F12:DMEM media supplemented with 5% horse serum (Thermo Fisher Scientific), 20 ng/ml human EGF (Sigma), 0.5 mg/ml hydrocortisone (Sigma), 100 ng/ml cholera toxin (Sigma) and 10 μg/ml recombinant human insulin (Sigma).

Generation of Knockout Cell Lines: 10⁶ cells were electroporated using the Lonza 4D-Nucleofector X Unit (MDA-MB-453) or Lonza Nucleofector 2b Device (BT-474, BC-1, JSC-1) using programs DK-100 (MDA-MB-453), X-001 (BT-474), or T-001 (BC-1, JSC-1) in buffer SF+18% supplement (MDA-MB-453) or 80% Solution 1 (125 mM Na₂HPO₄·7H₂O, 12.5 mM KCl, acetic acid to pH=7.75) and 20% Solution 2 (55 mM MgCl₂) (BT-474, BC-1, JSC-1) and 9 μg (UNG, SMUG1, REV1) or 10 μg (A3A, A3B) of pU6-sgRNA_CBh-Cas9-T2A-mCherry plasmid DNA. Electroporated cells were plated into 10 cm dishes and the media was changed after 24 h. mCherry positive cells were single-cell sorted into 96-well plates by FACS using FACSAria (BD Biosciences). To generate the APOBEC3B KO in JSC-1 cells, 106 cells were transfected with 10 μg pU6-sgA3B_CBh-Cas9-T2A-mCherry DNA using Lipofectamine 3000 reagent (ThermoFisher Scientific cat. #L3000015). Cells were plated for 48 h, after which mCherry positive cells were bulk sorted, grown, and subcloned by limiting dilution.

Knockout Screening and Validation by PCR: CRISPR KO Clone Screening. Cells were pelleted and their genomic DNA isolated using the Cell Monolayer protocol of the Zymo Research Genomic DNA Isolation Kit (cat. #ZD3025). Purified genomic DNA for CRISPR/Cas9 knockout screens was amplified using Touchdown PCR. Each PCR reaction consisted of: 7.4 μL ddH₂O, 1.25 μL 10×PCR buffer (166 mM NH₄SO₄, 670 mM Tris base (pH 8.8), 67 mM MgCl₂, 100 mM β-mercaptoethanol), 1.5 μL 10 mM dNTPs, 0.75 μL DMSO, 0.25 μL forward and reverse primers (10 μM each), 0.1 μL Platinum Taq DNA Polymerase (Invitrogen, cat. #10966083), and 1 μL genomic DNA.

PCR for Sanger Sequencing: PCR reactions for Sanger Sequencing were performed using the Invitrogen Platinum Taq DNA Polymerase (Invitrogen, cat. #10966083) protocol. 25 ng of genomic DNA was used for each reaction. DNA from PCR reactions was purified from agarose gels using the Invitrogen PureLink Quick Gel Extraction Kit (Invitrogen, cat. #K210012). Gel-purified DNA was cloned using the TOPO TA Cloning Kit for Sequencing (Invitrogen, cat. #450030) and grown on LB-Amp plates and colonies were selected for sequencing (Genewiz).

RNA Isolation and Quantitative PCR: Cells were pelleted, and their RNA isolated using the Zymo Research Quick-RNA Miniprep Kit (cat. #R1054). RNA was quantified and converted to cDNA using the Invitrogen SuperScript IV First-Strand Synthesis System (cat. #18091050). cDNA synthesis reactions were performed using 2 μL of 50 ng/μL random hexamers, 2 μL of 10 mM dNTPs, 4 μg RNA, and DEPC-treated water to a volume of 26 μL. The mixture was heated at 65° C. for 5 minutes, then cooled on ice for 5 minutes. Primers, probes, and cycling conditions were adapted from published methods (Refsland et al. 2010).

Immunoblotting: Cells were lysed in 2× sample buffer (100 mM TrisHCl, pH 6.8, 4% SDS, 10% β-mercaptoethanol). The whole-cell lysate was subjected to SDS-polyacrylamide gel electrophoresis on NuPAGE 4-12% Bis-Tris gradient gels (Novex Life Technologies) and proteins were transferred onto a nitrocellulose membrane (Millipore).

Cells were lysed in RIPA buffer [(150 mM NaCl, 50 mM Tris-HCl pH=8, 1% NP-40, 0.5% sodium deoxycholate, 0.1% SDS, Pierce Protease Inhibitor Tablet, EDTA free] or sample buffer (125 mM Tris-HCl pH 6.8, 1 M β-mercaptoethanol, 4% SDS, 20% glycerol, 0.02% bromophenol blue). Quantification of RIPA extracts was performed using the Thermo Scientific Pierce BCA Protein Assay kit. Protein transfer was performed via wet transfer using 1×Towbin buffer (25 mM Tris, 192 mM glycine, 0.01% SDS, 20% methanol) and nitrocellulose membrane. Blocking was performed in 5% milk in 1×TBST (19 mM Tris, 137 mM NaCl, 2.7 mM KCl, and 0.1% Tween-20) for 1 h at room temperature (RT). The following antibodies were used: anti-APOBEC3A (see below; WB 1:500), anti-APOBEC3B (Abeam; ab184990; WB 1:500), anti-SMUG1 (Abeam; ab192240; WB 1:1,000), anti-UNG (abeam; ab109214; WB 1:1,000), anti-GFP (Santa Cruz; sc-9996; WB 1:1,000), anti-3-actin (Abeam; ab8224; WB 1:3,000), anti-3-actin (Abeam, ab8227; WB 1:3,000); anti-Mouse IgG HRP (Thermo Fisher Scientific; 31432; 1:10,000), and anti-Rabbit IgG HRP (SouthernBiotech; 6441-05; 1:10,000).

APOBEC3 monoclonal antibody generation: Residues 1-29 (N1-term) or 13-43 (N2-term) from APOBEC3A and residues 354-382 (C-term) from APOBEC3B and were used to create three peptide immunogens (EZBiolab). Five mice were given three injections using Keyhole-Limpet-Hemocyanin (KLH)-conjugated peptides over the course of 12 weeks (MSKCC Antibody and Bioresource Core). Test bleeds from the mice were screened for anti-APOBEC3A titers by ELISA against APOBEC3A peptides conjugated to BSA. Mice showing positive anti-APOBEC3A immune responses were selected for final immunization boost before their spleens were harvested for B-cell isolation and hybridoma production. Hybridoma fusions of myeloma (SP2/IL6) cells and viable splenocytes from the selected mice were performed by MSKCC Antibody and Bioresource Core. Cell supernatants were screened by APOBEC3A ELISA. The strongest positive hybridoma pools were subcloned by limiting dilution to generate monoclonal hybridoma cell lines. Hybridomas 04A04 and 01D05 were expanded then grown in 1% FBS medium for XX. This medium was clarified by centrifugation to remove cells and then passed over a Protein G column (04A04) or Protein A column (01D05) to bind mAb. The resulting mAb was eluted in PBS (04A04) or 100 mM NaCitrate pH 6, 150 mM NaCl buffer (01D05).

In vitro DNA deaminase activity assay: Deamination activity assays were performed as described (Stenglein et al. 2010). Briefly, 1 million cells were pelleted and lysed in buffer (25 mM HEPES, 150 mM NaCl, 1 mM EDTA, 10% glycerol, 0.5% Triton-X, 1× protease inhibitor), sheared through a 28½-gauge syringe, then cleared by centrifugation at 13,000×g for 10 minutes at 4° C. Deaminase reactions (16.5 μl cell extracts with 2 μl UDG buffer (NEB), 0.5 μl RNase A (20 mg/ml), 1 μl 1 μM probe (linear=5′IRD800/ATTATTATTATTATTATTATTTCATTTATTTATTTATTTA (SEQ ID NO: 5) or hairpin=5′IRD800/ATTATTATTATTGCAAGCTGTTCAGCTTGCTGAATTTATT (SEQ ID NO: 6)), and 0.3 μl UDG (NEB)) were incubated at 37° C. for 2 hours followed by addition of 2 μl 1M NaOH and 15 minutes at 95° C. to cleave abasic sites. Reactions were then neutralized with 2 μl 1 M HCl, terminated by adding 20 μl urea sample buffer (90% formamide+EDTA), and separated on a pre-warmed 15% acrylamide/urea gel in 1×TBE buffer at 60° C. for 70 minutes at 100V to monitor DNA cleavage. Gels were imaged by Odyssey Infrared Imaging System (Li-COR) and quantified via ImageJ.

Comparison of APOBEC3-associated mutational signatures in cell line and primary cancer data: Annotations of mutational signatures across 1,001 human cancer cell lines and 2,710 primary cancers from multiple cancer types were published previously (Petljak et al. 2019). Where possible, cancer and cell line cancer classes were matched. Eventually, 780 cell lines and 1843 primary cancers from matching cancer types were used in analyses presented in FIG. 1B. The signature annotation is available in previously published Table S3 of (Petljak et al. 2019).

Whole-genome sequencing: Genomic DNA was extracted from a total of 136 individual clones using the DNeasy Blood and Tissue Kit (QIAGEN) and quantified with Biotium Accuclear Ultra high sensitivity dsDNA Quantitative kit using Mosquito LV liquid platform, Bravo WS and BMG FLUOstar Omega plate reader. Samples were diluted to 200 ng/120 μl using Tecan liquid handling platform, sheared to 450 bp using a Covaris LE220 instrument and purified using Agencourt AMPure XP SPRI beads on Agilent Bravo WS. Library construction (ER, A-tailing and ligation) was performed using ‘NEB Ultra II custom kit’ on an Agilent Bravo WS automation system. PCR was set up using Agilent Bravo WS automation system, KapaHiFi Hot start mix and IDT 96 iPCR tag barcodes or unique dual indexes (UDI, Ilumina). PCR included 6 standard cycles: 1) incubation at 95° C. 5 mins; 2) incubation at 98° C. 30 s; 3) incubation at 65° C. 30 s; 4) incubation at 72° C. 1 min; 5) cycle from 2, 5 more times; 6) incubation at 72° C. 10 mins. Post-PCR plates were purified with Agencourt AMPure XP SPRI beads on Beckman BioMek NX96 liquid handling platform. Libraries were quantified with Biotium Accuclear Ultra high sensitivity dsDNA Quantitative kit using Mosquito LV liquid handling platform, Bravo WS and BMG FLUOstar Omega plate reader, pooled in equimolar amounts on a Beckman BioMek NX-8 liquid handling platform and normalized to 2.8 nM ready for cluster generation on a c-BOT. Pooled samples were loaded on the Illumina Hiseq X platform using 150 PE run lengths and sequenced to approximately 30× coverage. Sequencing reads were aligned to the reference human genome (GRCh37) using Burrows-Wheeler Alignment (BWA)-MEM (https://github.com/cancerit/PCAP-core). Unmapped, non-uniquely mapped reads and duplicate reads were excluded from further analyses.

Mutation calling: Somatic single base substitutions (SBS) were discovered using CaVEMan (https://github.com/cancerit/cgpCaVEManWrapper) (Jones et al. 2016), with major and minor copy number options set to, respectively, 5 and 2, to maximize discovery sensitivity. Rearrangements were identified with the BRASS algorithm (https://github.com/cancerit/BRASS). Sequences of the corresponding parent clones were used as reference genomes to discover mutations in individual daughter clones, whereas an unrelated normal human genome (Petljak et al. 2019) was used as a reference to discover mutations in parent clones. Mutations shared between parent clones (see below) were used to derive proxies for the mutational catalogues of bulk cell lines in FIG. 1C. Rearrangements were retained only if identified as absent from the reference sequences by BRASS. SBS discovered with CaVEMan were filtered over the two additional steps: first, to remove the low-quality loci and second, to ensure that the final mutational catalogues from daughter clones retained exclusively somatic mutations acquired during the examined in vitro periods and that the mutational catalogues from parent clones retained predominantly somatic mutations acquired in individual parent cell lineages prior to the examined in vitro periods spanning the two cloning events.

First, only SBS flagged as ‘PASS’ by Caveman when analyzed across the panel of 98 unmatched normal samples (github.com/cancerit/cgpCaVEManWrapper) (Jones et al. 2016) were considered, removing large proportions of mapping and sequencing artefacts, as well as the common germline variation presenting across the 98 healthy samples (Jones et al. 2016). Four post-hoc filters were applied to ‘PASS’ variants to further remove sequencing and mapping artefacts that occur with XTEN and BWA-mem-aligned data and to ensure that the mutation loci were well covered in the reference sequences. ‘PASS’ mutations were removed if (Filter 1) the median alignment score (ASMD) of mutation-reporting reads was less or equal to 140; (Filter 2) the mutation locus had the clipping index (CLPM) greater than 0; (Filter 3) the mutation locus was covered by 20 or fewer reads in the reference samples used in comparisons; and (Filter 4) less than two sequencing reads of opposite directions reported the mutation.

Second, all mutation loci that passed the filters above across all available clones obtained from the matching cell lines were genotyped. cgpVAF was used to count the number of mutant and wild type reads across individual clones (github.com/cancerit/vafCorrect) and mutations from each parent or daughter clone that were found at cumulative VAF of >5% across >10% of clones from other parental lineages were removed (Filter 5). Mutations presenting at other clones below these cut-offs were determined false-positive calls upon manual inspection of individual reads and were thus retained. In mutational catalogues from parent clones, this step removed the majority of the germline mutations and a smaller proportion of somatic mutations shared between parent clones, thus retaining predominantly somatic mutations acquired in individual parent cell lineages prior to the examined in vitro periods spanning the two cloning events. Such likely pre-existent germline and somatic mutations identified were accumulated across the related parent clones into mutational catalogues of bulk cell lines (FIG. 1C). In mutational catalogues from daughter clones, the same filter removed mutations which presented across clones from other parental lineages and were thus likely acquired before examined in vitro periods but were not captured in the corresponding reference sequences. The percentages of mutations removed with this filter also represent the upper-level estimates of the remaining false-positive de novo SBS calls in mutational catalogues from daughter clones, which would not have been captured in the reference sequences and would be designated as de novo. Such mutations would not be removed by filtering against other parental lineages, but their estimated proportions do not affect results and are generally minor (median ˜2.5%). Finally, while this filter removes most of the germline and the preexisting variation, a smaller proportion of the removed mutations may have arisen independently across multiple parental lineages at the hairpin loci that are hotspots for APOBEC-3 associated mutagenesis. However, such hotspot loci are extremely rare and would affect negligible numbers of mutations compared to much more prevalent genome-wide APOBEC3-associated mutations that occur outside of such loci (Buisson et al. 2019).

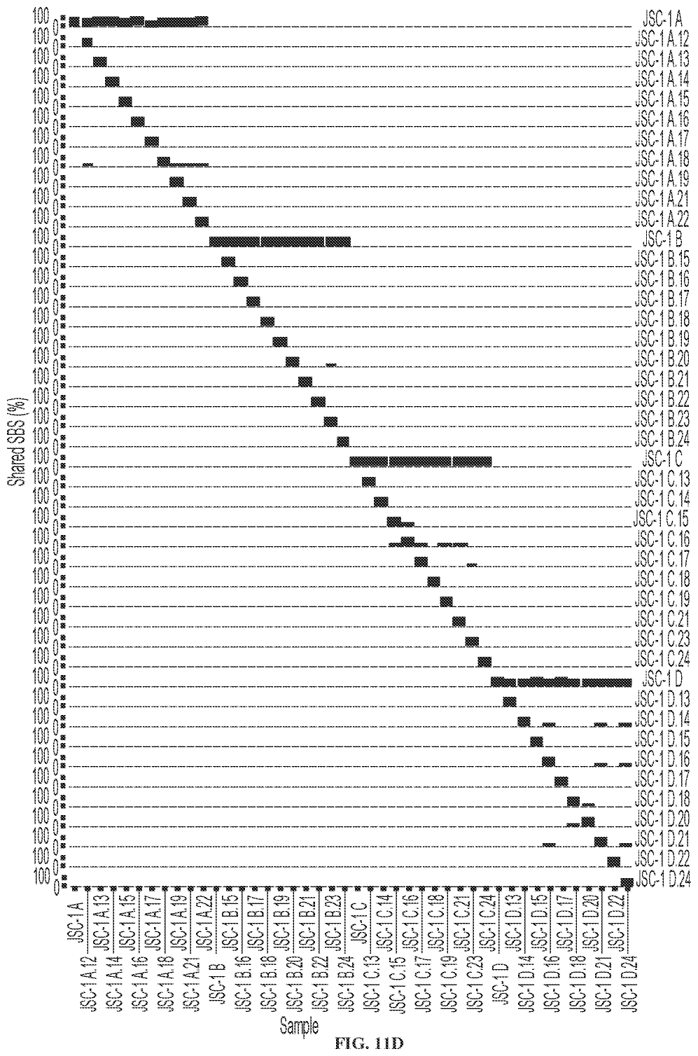

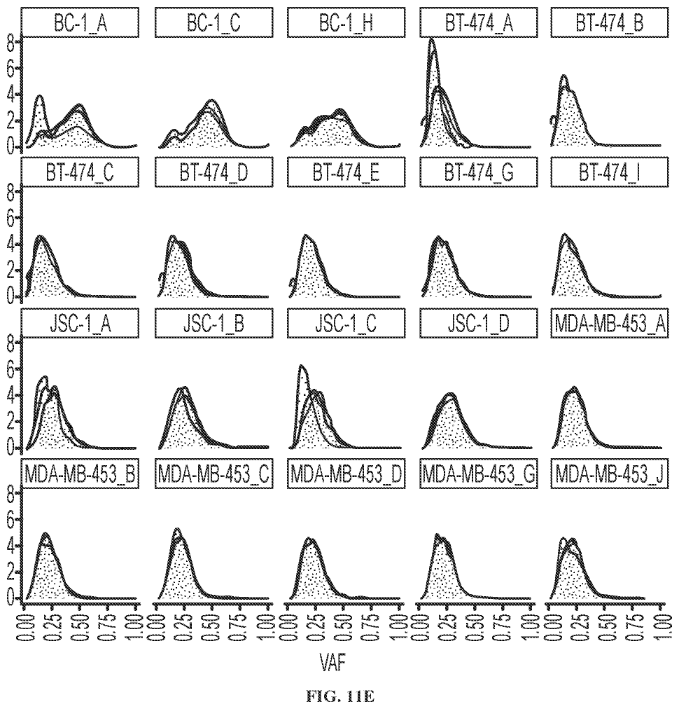

Validation of parent-daughter allocations: Genotyping of mutation loci across all clones revealed that, occasionally, a large proportion of mutations absent from the parent clones, and thus postulated to be acquired de novo in culture, was shared between some or all of their daughters (e.g., FIG. 11C, BC-1_C lineage daughter clones). To exclude the possibility that high proportions of shared mutations stem from allocations of the relevant daughters to the wrong parents, it was investigated whether the expected CRISPR-edits were detected in the genome sequences from all such daughters and whether such shared mutations were present in any available clones from other parental lineages. This originally revealed a swap between two lineages and a couple of clones from JSC-1 cell line. A few clones that exhibited a higher level of sharedness were not resolved in this way (e.g., daughters from BC-1_C lineage; BC-1_H.3 and BC-1_H.8). To exclude the possibility of clone cross-contaminations, in which case VAF of shared mutations would be lower than VAFs of other clonal mutations in some clones, it was confirmed that the VAF distributions of shared mutations followed those of other clonal mutations.

In the absence of sample swaps and putative contaminations, high proportions of clonal mutations that are shared between some of the related daughters and absent from their corresponding parents indicate that these mutations were indeed acquired de novo. Such daughters were overall rare and most likely established from the common subclone that arose at some point during the cultivation of the parent clone, after its DNA was already extracted.

Validation of clonal sample origins: To ensure that samples were clonal and single-cell-derived, proportions of the variant-reporting reads were examined (equivalent to variant allele fraction, VAF) at the mutation loci. Consistent with the polyploid background of most cell lines under investigation (Petljak et al. 2019), VAF distributions often deviated from the average of ˜50% expected for clonal heterozygous somatic mutations occurring in a diploid genome. The largely unimodal VAF distributions validated the clonal origin of the majority of the samples. Bimodal VAF distributions were observed in several clones. However, in all cases, at least one of the peaks followed the VAF distribution of other clonal samples from the same cell line, indicating that the other peak presenting in some clones likely originates from the sub-clonal evolution taking place in culture after the relevant single cells were isolated. Such instances were overall rare, but most common in the clones from the BC-1 cell line.

Sequence context-based classification of single base substitutions: SigProfilerMatrixGenerator (python v.1.1; github.com/AlexandrovLab/SigProfilerMatrixGenerator) (Bergstrom et al. 2019) was used to categorize SBSs into three separate sequence-context based classifications, which were used in analyses of mutation enrichments at APOBEC3-associated target motifs, and mutational signatures analyses. The algorithm allocates each SBS to (1) one of the 6-class categories (C>A, C>G, C>T, T>A, T>C and T>G) in which the mutated base is represented by the pyrimidine of the base pair; (2) to one of the 96-class categories (in which each of 6-class mutation types is further split into 16 subcategories baked on the flanking 5′ and 3′ bases); (3) and to one of the 1,536-class categories (in which each of 6-class mutation types is further split into 256 subcategories based on two flanking bases 5′ and 3′ to the mutated base).

Enrichment of APOBEC3-associated mutations at target motifs: Once SBSs were allocated to their sequence context classes as described, whereby the mutated base is represented by the pyrimidine base of the base pair, C>T and C>G base substitutions at TCN (N is any mutation) contexts which brand APOBEC3-associated SBS2 and SBS13 signatures were classified as ‘APOBEC’, whereas C>T and C>G substitutions at other contexts were classified as ‘OTHER’. C>A substitutions were excluded for simplicity, because some of the C>A mutations have been attributed to both APOBEC mutagenesis, as well as other mutational processes commonly arising during in vitro cell cultivation (Petljak et al. 2019). Enrichment of ‘APOBEC’ mutations was then investigated in the target sequence motifs associated with APOBEC mutagenesis previously, including specific pentanucleotide motifs (Chan et al. 2015) across all clones.

Enrichment of APOBEC3-associated mutations at trinucleotide and pentanucleotide motifs: Enrichment of APOBEC3-associated mutations was compared across the pentanucleotide motifs that were previously associated with APOBEC3A (YTCN and YTCA, where Y is a pyrimidine base) and APOBEC3B activities (RTCN and RTCA, where R is a purine base) in yeast overexpression systems (Chan et al. 2015). Relevant APOBEC3-associated trinucleotide and pentanucletide sequence motifs were quantified with sequence_utils (v.1.1.0, github.com/cancerit/sequence_utils/releases/tag/1.1.0; (github.com/cancerit/sequence_utils/wiki #sequence-context-of-regions-processed-by-caveman) across human autosomal chromosomes (GRCh37) and by excluding the regions not considered by the CaVEMan algorithm in detecting SBS. Middle base pair of each reference pentanucleotide sequence was considered a putative mutation target and the sequence context surrounding it was quantified using the DNA strand belonging to the pyrimidine base of the target base-pair, giving rise to a total of 96 trinucleotide and 512 possible pentanucleotide contexts that were quantified across both DNA strands (e.g., AGT trinucleotide is reported as ACT; AAGCA pentanucleotide is reported as TGCTT; middle ‘target’ bases underlined). Enrichment of ‘APOBEC’ mutations at the pentanucleotide motifs of interest was calculated as described previously (Petljak et al. 2019; Chan et al. 2015). For example, to calculate enrichment (E) of ‘APOBEC’ mutations at RTCN sites the following was used: ERTCN=(MutAPOBEC(RTCN)/ConRTCN)/(MutAPOBEC(TCN)/ConTCN).

MutAPOBEC(TCN) is the total number of ‘APOBEC’ mutations (C>G and C>T mutations at TCN contexts) in autosomal chromosomes; MutAPOBEC(RTCN) is the sum of ‘APOBEC’ mutations at RTCN contexts in autosomal chromosomes; whereas ConTCN and ConRTCN represent the total number of TCN and RTCN contexts available among the regions considered by Caveman when calling mutations across the autosomal chromosomes. As described, both DNA strands are considered, but the mutation types and target motifs are reported based on the strand of the pyrimidine base of the target base pair.

Mutational signatures analysis: Mutational signatures analyses were performed using the SigProfilerExtractor tool (v. 1.0.17; github.com/AlexandrovLab/SigProfilerExtractor) (Islam et al. 2021), which is a method based on nonnegative matrix factorization (NMF) for de novo extraction of mutational signatures from a given matrix of SBS types. SBS were classified into 96 classes based on their trinucleotide sequence contexts (see ‘Sequence context-based classification of single base substitutions’). The tool was used over 500 iterations to identify profiles of mutational signatures operative across a total of 815,923 genome-wide mutations identified across 4 bulk cell lines and their corresponding 136 daughter and parent clones.

Mutational signatures were extracted de novo and subsequently mapped to the known COSMIC Mutational Signatures of cleaner patterns (v3, //cancer.sanger.ac.uk/cosmic/signatures). Activities of identified COSMIC mutational signatures were quantified in each clone as part of the factorization of the input 96-SBS channel matrices, whereby numbers of SBS mutations belonging to each identified signature were quantified in the genome of each sample. All the relevant outputs from SigProfilerExtractor include profiles of de novo extracted signatures, metrics related to mapping of de novo signatures to COSMIC signature profiles and per-sample activity estimations.

Kataegis identification: Kataegis, or foci of localized hypermutation (Nik-Zainal et al., 2012a), were quantified in 136 whole-genome sequenced parent and daughter clones. The relevant focus was defined as a cluster of 5 or more consecutive APOBEC3-associated mutations (C>A, C>T, and C>G substitutions at TCN trinucleotides), exhibit strand-coordination and have an average inter-mutation distance of <7,500 bases. While the approach may miss some foci, sensitivity of detection was sacrificed to obtain higher predictive value of kataegis foci.

Identification of clustered mutations: To detect clustered single base substitutions, a sample-dependent inter-mutational distance (IMD) cutoff was derived, which is unlikely to occur by chance given the mutational pattern and mutational burden of each clone. To derive a background model reflecting the distribution of mutations that one would expect to observe by chance, SigProfilerSimulator (v1.1.2) was used to randomly simulate the mutations in each clone across the genome (Bergstrom et al. 2020). Specifically, the model was generated to maintain the +/−1 bp sequence context for each substitution, the strand coordination including the transcribed or untranscribed strand within genic regions (Bergstrom et al. 2020) and the total number of mutations across each chromosome for a given sample. All single base substitutions were randomly simulated 100 times and used to calculate the sample-dependent IMD cutoff so that 90% of mutations below this threshold were clustered with respect to the simulated model (i.e., not occurring by chance with a q-value <0.01). Further, the heterogeneity in mutation rates across the genome and the variances in clonality or copy-number were considered by correcting for mutation rich regions present in 10 Mb-sized windows and by using a threshold for the difference in variant allele frequencies between subsequent substitutions in a clustered event (variant allele frequency difference <0.10). Subsequently, the clustered mutations were subclassified into specific categories of events: (i) doublet substitutions; two adjacent mutations with consistent variant allele frequencies; (ii) extended multi-base substitutions; previously termed omikli events (Mas-Ponte et al. 2020) that reflect any two mutational events greater than 1 bp and less than the sample-dependent IMD cutoff with consistent variant allele frequencies; (iii) large mutational events; previously termed kataegi (Nik-Zainal et al. 2012) with three or more mutational events greater than 1 bp and less than the sample-dependent IMD cutoff with consistent variant allele frequencies. Lastly, statistical comparisons across clones were performed using a Mann-Whitney U test.

### REFERENCES

Nik-Zainal, S. et al. Mutational processes molding the genomes of 21 breast cancers. Cell 149, 979-993 (2012).
Roberts, S. A. et al. Clustered mutations in yeast and in human cancers can arise from damaged long single-strand DNA regions. Mol. Cell 46, 424-435 (2012).
Petljak, M. Maciejowski, J. Molecular Origins of APOBEC-Associated Mutations in Cancer. DNA Repair 102905 (2020).
Petljak, M. et al. Characterizing Mutational Signatures in Human Cancer Cell Lines Reveals Episodic APOBEC Mutagenesis. Cell 176, 1282-1294.e20 (2019).
Alexandrov, L. B. et al. The repertoire of mutational signatures in human cancer. Nature 578, 94-101 (2020).
Alexandrov, L. B. et al. Signatures of mutational processes in human cancer. Nature 500, 415-421 (2013).
Helleday, T., Eshtad, S. Nik-Zainal, S. Mechanisms underlying mutational signatures in human cancers. Nat. Rev. Genet. 15, 585-598 (2014).
Chan, K. et al. An APOBEC3A hypermutation signature is distinguishable from the signature of background mutagenesis by APOBEC3B in human cancers. Nat. Genet. 47, 1067-1072 (2015).
Taylor, B. J. et al. DNA deaminases induce break-associated mutation showers with implication of APOBEC3B and 3A in breast cancer kataegis. Elife 2, e00534 (2013).
Granadillo Rodriguez, M., Flath, B. Chelico, L. The interesting relationship between APOBEC3 deoxycytidine deaminases and cancer: a long road ahead. Open Biol. 10, 200188 (2020).
Green, A. M. Weitzman, M. D. The spectrum of APOBEC3 activity: From anti-viral agents to anti-cancer opportunities. DNA Repair 83, 102700 (2019).
Burns, M. B. et al. APOBEC3B is an enzymatic source of mutation in breast cancer. Nature 494, 366-370 (2013).
Burns, M. B., Temiz, N. A. Harris, R. S. Evidence for APOBEC3B mutagenesis in multiple human cancers. Nat. Genet. 45, 977-983 (2013).
Roberts, S. A. et al. An APOBEC cytidine deaminase mutagenesis pattern is widespread in human cancers. Nat. Genet. 45, 970-976 (2013).
Cortez, L. M. et al. APOBEC3A is a prominent cytidine deaminase in breast cancer. PLoS Genet. 15, e1008545 (2019).
Buisson, R. et al. Passenger hotspot mutations in cancer driven by APOBEC3A and mesoscale genomic features. Science 364, (2019).
Nik-Zainal, S. et al. Association of a germline copy number polymorphism of APOBEC3A and APOBEC3B with burden of putative APOBEC-dependent mutations in breast cancer. Nat. Genet. 46, 487-491 (2014).
Starrett, G. J. et al. The DNA cytosine deaminase APOBEC3H haplotype I likely contributes to breast and lung cancer mutagenesis. Nat. Commun. 7, 12918 (2016).
Middlebrooks, C. D. et al. Association of germline variants in the APOBEC3 region with cancer risk and enrichment with APOBEC-signature mutations in tumors. Nat. Genet. 48, 1330-1338 (2016).
ICGC/TCGA Pan-Cancer Analysis of Whole Genomes Consortium. Pan-cancer analysis of whole genomes. Nature 578, 82-93 (2020).
Venkatesan, S. et al. Perspective: APOBEC mutagenesis in drug resistance and immune escape in HIV and cancer evolution. Ann. Oncol. 29, 563-572 (2018).
Swanton, C., McGranahan, N., Starrett, G. J. Harris, R. S. APOBEC Enzymes: Mutagenic Fuel for Cancer Evolution and Heterogeneity. Cancer Discov. 5, 704-712 (2015).
Green, A. M. et al. Cytosine Deaminase APOBEC3A Sensitizes Leukemia Cells to Inhibition of the DNA Replication Checkpoint. Cancer Res. 77, 4579-4588 (2017).
Buisson, R., Lawrence, M. S., Benes, C. H. Zou, L. APOBEC3A and APOBEC3B Activities Render Cancer Cells Susceptible to ATR Inhibition. Cancer Res. 77, 4567-4578 (2017).
Law, E. K. et al. The DNA cytosine deaminase APOBEC3B promotes tamoxifen resistance in ER-positive breast cancer. Sci. Adv. 2, e1601737 (2016).
Driscoll, C. B. et al. APOBEC3B-mediated corruption of the tumor cell immunopeptidome induces heteroclitic neoepitopes for cancer immunotherapy. Nat. Commun. 11, 790 (2020).
Nikkilä, J. et al. Elevated APOBEC3B expression drives a kataegic-like mutation signature and replication stress-related therapeutic vulnerabilities in p53-defective cells. Br. J. Cancer 117, 113-123 (2017).
Olson, M. E., Harris, R. S. Harki, D. A. APOBEC Enzymes as Targets for Virus and Cancer Therapy. Cell Chemical Biology vol. 25 36-49 (2018).
Harris, R. S., Petersen-Mahrt, S. K. Neuberger, M. S. RNA editing enzyme APOBEC1 and some of its homologs can act as DNA mutators. Mol. Cell 10, 1247-1253 (2002).
Jarvis, M. C., Ebrahimi, D., Temiz, N. A. Harris, R. S. Mutation Signatures Including APOBEC in Cancer Cell Lines. JNCI Cancer Spectr. 2, (2018).
Alexandrov, L. B. et al. Clock-like mutational processes in human somatic cells. Nat. Genet. 47, 1402-1407 (2015).
Grolleman, J. E. et al. Mutational Signature Analysis Reveals NTHL1 Deficiency to Cause a Multi-tumor Phenotype. Cancer Cell 35, 256-266.e5 (2019).
Rouhani, F. J. et al. Mutational History of a Human Cell Lineage from Somatic to Induced Pluripotent Stem Cells. PLoS Genet. 12, e1005932 (2016).
Pilati, C. et al. Mutational signature analysis identifies MUTYH deficiency in colorectal cancers and adrenocortical carcinomas. J. Pathol. 242, 10-15 (2017).
van Loon, B., Markkanen, E. Hubscher, U. Oxygen as a friend and enemy: How to combat the mutational potential of 8-oxo-guanine. DNA Repair 9, 604-616 (2010).
Leonard, B. et al. APOBEC3B upregulation and genomic mutation patterns in serous ovarian carcinoma. Cancer Res. 73, 7222-7231 (2013).
Nilsen, H. et al. Excision of deaminated cytosine from the vertebrate genome: role of the SMUG1 uracil-DNA glycosylase. EMBO J. 20, 4278-4286 (2001).
Doseth, B., Ekre, C., Slupphaug, G., Krokan, H. E. Kavli, B. Strikingly different properties of uracil-DNA glycosylases UNG2 and SMUG1 may explain divergent roles in processing of genomic uracil. DNA Repair 11, 587-593 (2012).
Masuda, K. et al. A critical role for REV1 in regulating the induction of C: G transitions and A: T mutations during Ig gene hypermutation. The Journal of Immunology 183, 1846-1850 (2009).
Sale, J. E., Lehmann, A. R. Woodgate, R. Y-family DNA polymerases and their role in tolerance of cellular DNA damage. Nat. Rev. Mol. Cell Biol. 13, 141-152 (2012).
Simpson, L. J. Rev1 is essential for DNA damage tolerance and non-templated immunoglobulin gene mutation in a vertebrate cell line. The EMBO Journal vol. 22 1654-1664 (2003).
Ross, A.-L. Sale, J. E. The catalytic activity of REV1 is employed during immunoglobulin gene diversification in DT40. Mol. Immunol. 43, 1587-1594 (2006).
Kim, J. et al. Somatic ERCC2 mutations are associated with a distinct genomic signature in urothelial tumors. Nat. Genet. 48, 600-606 (2016).
Maciejowski, J. et al. APOBEC3-dependent kataegis and TREX1-driven chromothripsis during telomere crisis. Nat. Genet. (2020) doi:10.1038/s41588-020-0667-5.
Nik-Zainal, S. et al. The life history of 21 breast cancers. Cell 149, 994-1007 (2012).
Chan, K. Gordenin, D. A. Clusters of Multiple Mutations: Incidence and Molecular Mechanisms. Annu. Rev. Genet. 49, 243-267 (2015).
Jalili, P. et al. Quantification of ongoing APOBEC3A activity in tumor cells by monitoring RNA editing at hotspots. Nat. Commun. 11, 2971 (2020).
Mayekar, M. K. et al. Targeted cancer therapy induces APOBEC fuelling the evolution of drug resistance. Cold Spring Harbor Laboratory 2020.12.18.423280 (2020) doi:10.1101/2020.12.18.423280.
Isozaki, H., Abbasi, A., Nikpour, N., Langenbucher, A. Su, W. APOBEC3A drives acquired resistance to targeted therapies in non-small cell lung cancer. bioRxiv (2021).
Caval, V., Suspene, R., Shapira, M., Vartanian, J.-P. Wain-Hobson, S. A prevalent cancer susceptibility APOBEC3A hybrid allele bearing APOBEC3B 3′UTR enhances chromosomal DNA damage. Nat. Commun. 5, 5129 (2014).
Iorio, F. et al. A Landscape of Pharmacogenomic Interactions in Cancer. Cell 166, 740-754 (2016).
Garnett, M. J. et al. Systematic identification of genomic markers of drug sensitivity in cancer cells. Nature 483, 570-575 (2012).
Petljak, M. et al. Characterizing Mutational Signatures in Human Cancer Cell Lines Reveals Episodic APOBEC Mutagenesis. Cell 176, 1282-1294.e20 (2019).
Forbes, S. A. et al. COSMIC: somatic cancer genetics at high-resolution. Nucleic Acids Res. 45, D777-D783 (2017).
Refsland, E. W. et al. Quantitative profiling of the full APOBEC3 mRNA repertoire in lymphocytes and tissues: implications for HIV-1 restriction. Nucleic Acids Res. 38, 4274-4284 (2010).
Stenglein, M. D., Burns, M. B., Li, M., Lengyel, J. Harris, R. S. APOBEC3 proteins mediate the clearance of foreign DNA from human cells. Nat. Struct. Mol. Biol. 17, 222-229 (2010).
Jones, D. et al. cgpCaVEManWrapper: Simple Execution of CaVEMan in Order to Detect Somatic Single Nucleotide Variants in NGS Data. Curr. Protoc. Bioinformatics 56, 15.10.1-15.10.18 (2016).
Buisson, R. et al. Passenger hotspot mutations in cancer driven by APOBEC3A and mesoscale genomic features. Science 364, (2019).
Bergstrom, E. N. et al. SigProfilerMatrixGenerator: a tool for visualizing and exploring patterns of small mutational events. BMC Genomics 20, 685 (2019).
Chan, K. et al. An APOBEC3A hypermutation signature is distinguishable from the signature of background mutagenesis by APOBEC3B in human cancers. Nat. Genet. 47, 1067-1072 (2015).
Islam, S. M. A., Ashiqul Islam, S. M. Alexandrov, L. B. Bioinformatic Methods to Identify Mutational Signatures in Cancer. Leukemia Stem Cells 447-473 (2021) doi:10.1007/978-1-0716-0810-4-28.
Bergstrom, E. N., Barnes, M., Martincorena, I. Alexandrov, L. B. Generating realistic null hypothesis of cancer mutational landscapes using SigProfilerSimulator. BMC Bioinformatics 21, 438 (2020).
Mas-Ponte, D. Supek, F. DNA mismatch repair promotes APOBEC3-mediated diffuse hypermutation in human cancers. Nat. Genet. 52, 958-968 (2020).
Nik-Zainal, S. et al. Mutational Processes Molding the Genomes of 21 Breast Cancers. Cell vol. 149 979-993 (2012).

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following embodiments.

What is claimed is:

1. A method of treating cancer in a subject in need thereof comprising inhibiting one or more APOBEC deaminases and/or one or more DNA repair proteins with an agent.

2. The method of claim 1, wherein the method comprises inhibiting one or more APOBEC deaminases with an agent.

3. The method of claim 2, wherein the APOBEC deaminase is APOBEC3A.

4. The method of claim 2, wherein the APOBEC deaminase is APOBEC3B.

5. The method of claim 1, wherein the method comprises inhibiting a DNA repair protein with an agent.

6. The method of claim 5, wherein the DNA repair protein is REV1.

7. The method of claim 1, wherein the method comprises inhibiting an APOBEC deaminase and a DNA repair protein with an agent.

8. The method of claim 7, wherein the APOBEC deaminase is APOBEC3A.

9. The method of claim 7, wherein the APOBEC deaminase is APOBEC3B.

10. The method of any one of claims 7-9, wherein the DNA repair protein is REV1.

11. The method of claim 7, wherein the method comprises inhibiting APOBEC3A and REV1 with an agent.

12. The method of any one of claims 1-11, wherein the agent is a small molecule, a protein, a peptide, or a nucleic acid.

13. The method of any one of claims 1-12, wherein the agent is an mRNA, an antisense RNA, an miRNA, an siRNA, an RNA aptamer, a double stranded RNA (dsRNA), a short hairpin RNA (shRNA), or an antisense oligonucleotide (ASO).

14. The method of any one of claims 1-13, wherein the agent is an siRNA.

15. The method of any one of claims 1-11, wherein the agent is an antibody or a fragment thereof.

16. The method of any one of claims 1-15, wherein the cancer is bladder cancer, cervical cancer, lung cancer, head and neck cancer, breast cancer, esophageal cancer, lymphoma, oral squamous cell carcinoma, uterine cancer, ovarian adenocarcinoma, pancreatic adenocarcinoma, stomach adenocarcinoma, or biliary adenocarcinoma.

17. The method of any one of claims 1-16, wherein the cancer is lung cancer.

18. The method of claim 17, wherein the lung cancer is lung adenocarcinoma or squamous cell carcinoma.

19. The method of any one of claims 1-16, wherein the cancer is breast cancer.

20. The method of any one of claims 1-16, wherein the cancer is B cell lymphoma.

21. A method of treating cancer in a subject in need thereof comprising inhibiting APOBEC3A with an agent.

22. The method of claim 21, wherein the agent is a small molecule, a protein, a peptide, or a nucleic acid.

23. The method of claim 21 or 22, wherein the agent is an mRNA, an antisense RNA, an miRNA, an siRNA, an RNA aptamer, a double stranded RNA (dsRNA), a short hairpin RNA (shRNA), or an antisense oligonucleotide (ASO).

24. The method of any one of claims 21-23, wherein the agent is an siRNA.

25. The method of claim 21 or 22, wherein the agent is an antibody or a fragment thereof.

26. The method of any one of claims 21-25, wherein the cancer is bladder cancer, cervical cancer, lung cancer, head and neck cancer, breast cancer, esophageal cancer, lymphoma, oral squamous cell carcinoma, uterine cancer, ovarian adenocarcinoma, pancreatic adenocarcinoma, stomach adenocarcinoma, or biliary adenocarcinoma.

27. The method of any one of claims 21-26, wherein the cancer is lung cancer.

28. The method of claim 27, wherein the lung cancer is lung adenocarcinoma or squamous cell carcinoma.

29. The method of any one of claims 21-26, wherein the cancer is breast cancer.

30. The method of any one of claims 21-26, wherein the cancer is B cell lymphoma.

31. A method of identifying a subject in need of a treatment for cancer who is likely to respond to an APOBEC3A inhibitor comprising:
(i) providing a sample from the subject; and
(ii) determining whether a mutational signature induced by APOBEC3A is present in the sample, wherein the subject is likely to respond to the APOBEC3A inhibitor if the mutational signature induced by APOBEC3A are present in the sample.

32. The method of claim 31, wherein the determining of step (ii) comprises:
(i) providing a set of primers comprising a first primer and a second primer to the sample, wherein the first primer binds to a region of the genome upstream of a mutational signature induced by APOBEC3A, and the second primer binds to a region of the genome downstream of the mutational signature induced by APOBEC3A;
(ii) amplifying the region of the genome between the first primer and the second primer; and
(iii) sequencing the amplified region of the genome.

33. A method of identifying a subject in need of a treatment for cancer who is likely to respond to an APOBEC3B inhibitor comprising:
(i) providing a sample from the subject; and
(ii) determining whether a mutational signature induced by APOBEC3B is present in the sample, wherein the subject is likely to respond to the APOBEC3B inhibitor if the mutational signature induced by APOBEC3B are present in the sample.

34. The method of claim 33, wherein the determining of step (ii) comprises:
(i) providing a set of primers comprising a first primer and a second primer to the sample, wherein the first primer binds to a region of the genome upstream of a mutational signature induced by APOBEC3B, and the second primer binds to a region of the genome downstream of the mutational signature induced by APOBEC3B;
(ii) amplifying the region of the genome between the first primer and the second primer; and
(iii) sequencing the amplified region of the genome.

35. A method of identifying a subject in need of a treatment for cancer who is likely to respond to an REV1 inhibitor comprising:
(i) providing a sample from the subject; and
(ii) determining whether a mutational signature induced by REV1 is present in the sample, wherein the subject is likely to respond to the REV1 inhibitor if the mutational signature induced by REV1 are present in the sample.

36. The method of claim 35, wherein the determining of step (ii) comprises:
(i) providing a set of primers comprising a first primer and a second primer to the sample, wherein the first primer binds to a region of the genome upstream of a mutational signature induced by REV1, and the second primer binds to a region of the genome downstream of the mutational signature induced by REV1;
(ii) amplifying the region of the genome between the first primer and the second primer; and
(iii) sequencing the amplified region of the genome.

37. The method of any one of claims 31-36, wherein the determining of step (ii) comprises whole-genome sequencing.

38. The method of any one of claims 31-36, wherein the determining of step (ii) comprises whole-exome sequencing.

39. The method of any one of claims 31-38, wherein the mutational signature is a single base substitution (SBS).

40. The method of claim 39, wherein the SBS is SBS1, SBS2, SBS5, SBS8_18_36, or SBS13.

41. The method of any one of claims 31-40, wherein the cancer is bladder cancer, cervical cancer, lung cancer, head and neck cancer, breast cancer, esophageal cancer, lymphoma, oral squamous cell carcinoma, uterine cancer, ovarian adenocarcinoma, pancreatic adenocarcinoma, stomach adenocarcinoma, or biliary adenocarcinoma.

42. The method of any one of claims 31-41, wherein the cancer is lung cancer.

43. The method of claim 42, wherein the lung cancer is lung adenocarcinoma or squamous cell carcinoma.

44. The method of any one of claims 31-41, wherein the cancer is breast cancer.

45. The method of any one of claims 31-41, wherein the cancer is B cell lymphoma.

46. A method of tracking mutagenesis induced by a gene of interest in a population of cells over time comprising:
(i) knocking out the gene of interest in a cell from the population of cells to create a knockout (KO) cell line;
(ii) selecting a first KO clone from the KO cell line;
(iii) selecting a first wild-type (WT) clone from the population of cells;
(iv) propagating the first WT clone and the first KO clone by cell culture a first time into a first WT population of cells and a first KO population of cells;
(v) selecting a second WT clone and a second KO clone from the first WT population of cells and the first KO population of cells;
(vi) propagating the second WT clone and the second KO clone selected in step (v) by cell culture a second time to produce a second WT population of cells and a second KO population of cells;
(vii) sequencing the DNA of the second WT population of cells and the second KO population of cells; and
(viii) comparing the mutations present in the second WT population of cells and the second KO population of cells.

47. The method of claim 46, wherein the knocking out of step (i) comprises transfecting a cell from the population of cells with a vector encoding a nuclease.

48. The method of claim 47, wherein the nuclease is a Cas9 nuclease.

49. The method of claim 47 or 48, wherein the vector also encodes a gRNA, wherein the sequence of a portion of the gRNA is complementary to a portion of the gene of interest.

50. The method of any one of claims 46-49, wherein the propagating of step (iv) is performed for more than 10 days.

51. The method of any one of claims 46-50, wherein the propagating of step (iv) is performed for 50-150 days.

52. The method of any one of claims 46-51, wherein the sequencing of step (vii) is whole-genome sequencing.

53. The method of any one of claims 46-52, wherein the gene of interest is an APOBEC deaminase.

54. The method of any one of claims 46-53, wherein the gene of interest is APOBEC3A.

55. The method of any one of claims 46-54, wherein the gene of interest is APOBEC3B.

56. The method of any one of claims 46-52, wherein the gene of interest is REV1.

57. The method of any one of claims 46-52, wherein the gene of interest is AID.

58. The method of any one of claims 46-52, wherein the gene of interest is UNG.

59. The method of any one of claims 46-58, wherein the population of cells comprises bladder cancer cells, cervical cancer cells, lung cancer cells, head and neck cancer cells, breast cancer cells, esophageal cancer cells, lymphoma cells, oral squamous cell carcinoma cells, uterine cancer cells, ovarian adenocarcinoma cells, pancreatic adenocarcinoma cells, pancreatic adenocarcinoma cells, stomach adenocarcinoma cells, or biliary adenocarcinoma cells.

60. The method of any one of claims 46-59, wherein the population of cells comprises lung cancer cells.

61. The method of claim 60, wherein the lung cancer cells comprise lung adenocarcinoma cells or squamous cell carcinoma cells.

62. The method of any one of claims 46-59, wherein the population of cells comprises breast cancer cells.

63. The method of any one of claims 46-59, wherein the population of cells comprises B cell lymphoma cells.

64. A cancer cell line comprising a population of APOBEC3A knockout cells.

65. The cancer cell line of claim 64, wherein the cells are bladder cancer cells, cervical cancer cells, lung cancer cells, head and neck cancer cells, breast cancer cells, esophageal cancer cells, lymphoma cells, oral squamous cell carcinoma cells, uterine cancer cells, ovarian adenocarcinoma cells, pancreatic adenocarcinoma cells, stomach adenocarcinoma cells, or biliary adenocarcinoma cells.

66. The cancer cell line of claim 64 or 65, wherein the cells are lung cancer cells.

67. The cancer cell line of claim 64 or 65, wherein the cells are breast cancer cells.

68. The cancer cell line of claim 67, wherein the cells are derived from the human breast cancer cell line BT-474 or MDA-MB-453.

69. The cancer cell line of claim 64 or 65, wherein the cells are lymphoma cells.

70. The cancer cell line of claim 69, wherein the cells are derived from the human B cell lymphoma cancer cell line BC-1 or JSC-1.

71. A cancer cell line comprising a population of APOBEC3B knockout cells.

72. The cancer cell line of claim 71, wherein the cells are bladder cancer cells, cervical cancer cells, lung cancer cells, head and neck cancer cells, breast cancer cells, esophageal cancer cells, lymphoma cells, oral squamous cell carcinoma cells, uterine cancer cells, ovarian adenocarcinoma cells, pancreatic adenocarcinoma cells, stomach adenocarcinoma cells, or biliary adenocarcinoma cells.

73. The cancer cell line of claim 71 or 72, wherein the cells are lung cancer cells.

74. The cancer cell line of claim 71 or 72, wherein the cells are breast cancer cells.

75. The cancer cell line of claim 74, wherein the cells are derived from the human breast cancer cell line BT-474 or MDA-MB-453.

76. The cancer cell line of claim 71 or 72, wherein the cells are lymphoma cells.

77. The cancer cell line of claim 76, wherein the cells are derived from the human B cell lymphoma cancer cell line BC-1 or JSC-1.

78. A cancer cell line comprising a population of REV1 knockout cells.

79. The cancer cell line of claim 78, wherein the cells are bladder cancer cells, cervical cancer cells, lung cancer cells, head and neck cancer cells, breast cancer cells, esophageal cancer cells, lymphoma cells, oral squamous cell carcinoma cells, uterine cancer cells, ovarian adenocarcinoma cells, pancreatic adenocarcinoma cells, stomach adenocarcinoma cells, or biliary adenocarcinoma cells.

80. The cancer cell line of claim 78 or 79, wherein the cells are lung cancer cells.

81. The cancer cell line of claim 78 or 79, wherein the cells are breast cancer cells.

82. The cancer cell line of claim 81, wherein the cells are derived from the human breast cancer cell line BT-474 or MDA-MB-453.

83. The cancer cell line of claim 78 or 79, wherein the cells are lymphoma cells.

84. The cancer cell line of claim 83, wherein the cells are derived from the human B cell lymphoma cancer cell line BC-1 or JSC-1.

85. An isolated monoclonal antibody derived from a peptide comprising the amino acid sequence MEASPASGPRHLMDPHIFTSNFNNGIGRH (SEQ ID NO: 1).

86. The isolated monoclonal antibody of claim 85, wherein the antibody is an anti-APOBEC3A/B/G antibody.

87. The isolated monoclonal antibody of claim 85, wherein the antibody is an anti-APOBEC3A antibody.

88. The isolated monoclonal antibody of any one of claims 85-87, wherein the antibody is a mouse antibody.

89. The isolated monoclonal antibody of any one of claims 85-87, wherein the antibody is a humanized antibody.

90. The isolated monoclonal antibody of any one of claims 85-87, wherein the antibody is a human antibody.

91. A protein comprising the antigen binding region of the isolated monoclonal antibody of any one of claims 85-90.

92. A method of screening for inhibitors of APOBEC3A comprising:
(i) propagating a population of cells in the presence and absence of a candidate APOBEC3A inhibitor; and
(ii) determining whether the frequency of a mutational signature induced by APOBEC3A is reduced in the presence of the candidate APOBEC3A inhibitor.

93. A method of screening for inhibitors of APOBEC3B comprising:
(i) propagating a population of cells in the presence and absence of a candidate APOBEC3A inhibitor; and
(ii) determining whether the frequency of a mutational signature induced by APOBEC3B is reduced in the presence of the candidate APOBEC3B inhibitor.

94. The method of claim 92 or 93, wherein the mutational signature comprises one or more single base substitutions (SBS).

95. The method of claim 94, wherein the single base substitutions are selected from the group consisting of SBS1, SBS2, SBS5, SBS8_18_36, and SBS13.

96. The method of any one of claims 92-95, wherein the population of cells comprises bladder cancer cells, cervical cancer cells, lung cancer cells, head and neck cancer cells, breast cancer cells, esophageal cancer cells, lymphoma cells, oral squamous cell carcinoma cells, uterine cancer cells, ovarian adenocarcinoma cells, pancreatic adenocarcinoma cells, stomach adenocarcinoma cells, or biliary adenocarcinoma cells.

97. The method of any one of claims 92-96, wherein the population of cells comprises lung cancer cells.

98. The method of claim 97, wherein the lung cancer cells are lung adenocarcinoma cells or squamous cell carcinoma cells.

99. The method of any one of claims 92-96, wherein the cells are breast cancer cells.

100. The method of any one of claims 92-96, wherein the cells are B cell lymphoma cells.

101. A method of treating cancer in a subject in need thereof comprising enhancing the activity of APOBEC3B.

102. The method of claim 101, wherein enhancing the activity of APOBEC3B comprises inducing expression of APOBEC3B.

103. The method of claim 101 or 102, wherein the cancer is bladder cancer, cervical cancer, lung cancer, head and neck cancer, breast cancer, esophageal cancer, lymphoma, oral squamous cell carcinoma, uterine cancer, ovarian adenocarcinoma, pancreatic adenocarcinoma, stomach adenocarcinoma, or biliary adenocarcinoma.

104. The method of any one of claims 101-103, wherein the cancer is lung cancer.

105. The method of claim 104, wherein the lung cancer is lung adenocarcinoma or squamous cell carcinoma.

106. The method of any one of claims 101-105, wherein the cancer is breast cancer.

107. The method of any one of claims 101-105, wherein the cancer is B cell lymphoma.

108. A method of treating cancer in a subject in need thereof comprising inhibiting REV1 with an agent.

109. The method of claim 108, wherein the agent is a small molecule, a protein, or a nucleic acid.

110. The method of claim 108 or 109, wherein the agent is an mRNA, an antisense RNA, an miRNA, an siRNA, an RNA aptamer, a double stranded RNA (dsRNA), a short hairpin RNA (shRNA), or an antisense oligonucleotide (ASO).

111. The method of any one of claim 108-110, wherein the agent is an siRNA.

112. The method of claim 108 or 109, wherein the agent is an antibody or a fragment thereof.

113. The method of any one of claims 108-112, wherein the cancer is bladder cancer, cervical cancer, lung cancer, head and neck cancer, breast cancer, esophageal cancer, lymphoma, oral squamous cell carcinoma, uterine cancer, ovarian adenocarcinoma, pancreatic adenocarcinoma, stomach adenocarcinoma, or biliary adenocarcinoma.

114. The method of any one of claims 108-113, wherein the cancer is lung cancer.

115. The method of claim 114, wherein the lung cancer is lung adenocarcinoma or squamous cell carcinoma.

116. The method of any one of claims 108-113, wherein the cancer is breast cancer.

117. The method of any one of claims 108-113, wherein the cancer is B cell lymphoma.

118. A method of screening for inhibitors of REV1 comprising:
(i) propagating a population of cells in the presence and absence of a candidate REV1 inhibitor; and
(ii) determining whether the frequency of a mutational signature induced by REV1 is reduced in the presence of the candidate REV1 inhibitor.

119. The method of claim 118, wherein the mutational signature induced by REV1 comprises one or more single base substitutions (SBS).

120. The method of claim 119, wherein the single base substitutions are selected from the group consisting of SBS1, SBS2, SBS5, SBS8_18_36, and SBS13.

121. The method of any one of claims 118-120, wherein the population of cells comprises bladder cancer cells, cervical cancer cells, lung cancer cells, head and neck cancer cells, breast cancer cells, esophageal cancer cells, lymphoma cells, oral squamous cell carcinoma cells, uterine cancer cells, ovarian adenocarcinoma cells, pancreatic adenocarcinoma cells, stomach adenocarcinoma cells, or biliary adenocarcinoma cells.

122. The method of any one of claims 118-121, wherein the population of cells comprises lung cancer cells.

123. The method of claim 122, wherein the lung cancer cells are lung adenocarcinoma cells or squamous cell carcinoma cells.

124. The method of any one of claims 118-121, wherein the population of cells comprises breast cancer cells.

125. The method of any one of claims 118-121, wherein the population of cells comprises B cell lymphoma cells.

126. A method of screening for a synthetic lethality associated with active APOBEC3A comprising propagating a population of WT cells and a population of ABOBEC3A KO cells in the presence of an agent capable of inhibiting the activity of a gene of interest, wherein a synthetic lethality is identified when the population of WT cells is able to propagate in the presence of the agent and the population of APOBEC3A KO cells is not able to propagate in the presence of the agent.

127. The method of claim 126, wherein the agent is an inhibitor or a gene of interest.

128. The method of claim 127, wherein the inhibitor is a small molecule inhibitor or an siRNA inhibitor.

129. The method of claim 126, wherein the agent is a Cas9 nuclease associated with a gRNA, wherein the sequence of a portion of the gRNA is complementary to a portion of the gene of interest.

130. A method of screening for a synthetic lethality associated with active APOBEC3B comprising propagating a population of WT cells and a population of ABOBEC3B KO cells in the presence of an agent capable of inhibiting the activity of a gene of interest, wherein a synthetic lethality is identified when the population of WT cells is able to propagate in the presence of the agent and the population of APOBEC3B KO cells is not able to propagate in the presence of the agent.

131. The method of claim 130, wherein the agent is an inhibitor of a gene of interest.

132. The method of claim 131, wherein the inhibitor is a small molecule inhibitor or an siRNA inhibitor.

133. The method of claim 130, wherein the agent is a Cas9 nuclease associated with a gRNA, wherein the sequence of a portion of the gRNA is complementary to a portion of the gene of interest.

134. A kit comprising the cancer cell line of any one of claims 64-84.

135. A kit comprising the isolated monoclonal antibody of any one of claims 85-90 or the protein of claim 91.
